(19) United States
(12) Patent Application Publication
(10) Pub. No.: US 2024/0082228 A1
(43) Pub. Date: Mar. 14, 2024

(54) SARS-COV-2 THERAPEUTICS

(71) Applicants: MODEL MEDICINES, INC., La Jolla, CA (US); SANFORD BURNHAM PREBYS MEDICAL DISCOVERY INSTITUTE, La Jolla, CA (US)

(72) Inventors: George NICOLA, La Jolla, CA (US); Sumit CHANDA, La jolla, CA (US); Daniel HADERS, La Jolla, CA (US); Monika RAMOS, La Jolla, CA (US)

(21) Appl. No.: 18/273,686

(22) PCT Filed: Jan. 21, 2022
(86) PCT No.: PCT/US2022/013404

§ 371 Date: Jul. 21, 2023

Related U.S. Application Data
(60) Provisional application No. 63/140,762, filed on Jan. 22, 2021.

(51) Int. Cl. A61K 31/439; A61K 31/506; A61K 31/53; A61K 45/06; A61P 31/14

(52) U.S. Cl. CPC A61K 31/439; A61K 31/506; A61K 31/53; A61K 45/06; A61P 31/14

(57) ABSTRACT

Disclosed herein include methods, compositions, and kits suitable for use in treatment and/or prevention of RNA viral infections. In some embodiments, the method comprises modulation of host factors related to RNA viral infection using compounds, compositions and kits disclosed herein. There are provided, some embodiments, compounds modulating host factors that govern SARS-CoV-2 replication. Disclosed herein include the use of compounds, compositions and kits disclosed herein to prevent and/or treat SARS-CoV-2 infection, for example by modulating the SARS-CoV-2 life cycle.

### RELATED APPLICATIONS

The application claims the benefit under 35 U.S.C. § 119(e) of U.S. Provisional Patent Application Ser. No. 63/140,762, filed Jan. 22, 2021, the content of this related application is incorporated herein by reference in its entirety for all purposes.

### BACKGROUND

### Field

The present disclosure generally relates to the use of drugs for the treatment of RNA viral infections. More specifically, the disclosure describes methods for the treatment of an RNA viral infection and/or treatment or prevention of symptoms of an RNA viral infection by administering pharmaceutical compositions or their analogues.

### Description of the Related Art

An RNA virus is a virus that has RNA (ribonucleic acid) as its genetic material. This nucleic acid is usually single-stranded RNA (ssRNA) but may be double-stranded RNA (dsRNA). Notable human diseases caused by RNA viruses include the common cold, influenza, SARS, coronaviruses, COVID-19, hepatitis C, hepatitis E, West Nile fever, Ebola virus disease, rabies, polio and measles.

A large respiratory outbreak originating from Wuhan, China in December 2019 is currently spreading across many countries globally. The infectious disease was determined to be caused by a newly identified human coronavirus, severe acute respiratory syndrome coronavirus 2 (SARS-CoV-2, which causes the pneumonia like disease named COVID-19). As of Dec. 22, 2020, there are approximately 76.3M confirmed cases of severe acute respiratory syndrome (SARS-CoV-2) globally (WHO). The main symptoms of this virus are cough, shortness of breath or difficulty breathing, fever, headache, sore throat, and loss of taste and/or smell. New symptoms caused by SARS-CoV-2 are surfacing frequently. For instance, a notably severe effect of the virus' infection in the respiratory tract is that it can induce Acute Respiratory Distress Syndrome (ARDS) in addition to general respiratory complications. Treatment that serves to block viral infection or attenuate symptoms of SARS-CoV-2 are of utmost interest.

SARS-CoV-2 is part of the genus Betacoronavirus and shares structural and sequence similarity with SARS-CoV and MERS-CoV. This novel coronavirus is an enveloped positive sense RNA virus. Its structure is mainly encompassed by a spike (S) glycoprotein, a small envelope (E) glycoprotein, membrane (M) glycoprotein, and a nucleocapsid (N) protein. The S Protein facilitates binding and fusion for host-cell entry. The S protein is composed of two subunits, S1 and S2, that require proteolytic activation by host enzymes furin and TMPRSS2. Once activated, the S1 subunit utilizes its receptor binding domain to recognize and bind to the host's angiotensin-converting enzyme 2 (ACE2) located in the type II alveolar cells of the respiratory tract. The S2 subunit contains fusion peptides that facilitate fusion of the viral and host membranes.

The pharmaceutical candidates described herein have been investigated in various other diseases. In view of the large volume of data from the clinical investigation of these pharmaceutical candidates, and deep understanding of their clinical behaviors, it is beneficial to determine if these pharmaceutical candidates can be used to treat and/or prevent other disorders, for example RNA viral infections. There is an urgent need or compositions and methods for preventing, delaying the onset of, or treating an inflammatory effect of an infection or a disease caused by a RNA virus (e.g., SARS-CoV-2). Treatments that serve to block viral infection, replication, and/or attenuate symptoms of SARS-CoV-2 are of utmost interest. As described herein, a workflow was initiated to identify drugs that may be repurposed to modulate host factors related to SARS-CoV-2 replication.

### SUMMARY

Disclosed herein include the use of pharmaceutical compositions and pharmaceutical composition analogues for the treatment or prevention of disorders related to the modulation of one or more receptors related to RNA viral infections, for example coronavirus infections (including the abnormal behavioral symptoms related to coronavirus infections).

Some embodiments provide methods for the treatment or prevention of RNA viral infections using any one or more of the compounds disclosed herein, including a compound from Tables 2-8, or an analogue thereof, or pharmaceutical compositions containing any one or more of the compounds disclosed herein, including a compound of Tables 2-8 or an analogue thereof, or pharmaceutical compositions containing one or more pro-drugs of any one or more of the compounds disclosed herein, including the compound of Tables 2-8 or an analogue thereof by, for example, modulation of receptor related to RNA viral infection. Without being limited by any particular theory, it is believe that the treatment comprises causing the chemical effectors of the receptor related to RNA viral infection, by agonizing or antagonizing the effect of receptor related to RNA viral infection, which can also be used in addition to a safe and effective amount of one or more other agents to prevent or treat related symptoms and conditions.

Provided herein includes a method for preventing or treating an RNA viral infection. In some embodiments, the method comprises administering to a subject having the RNA viral infection or having a risk (e.g., a high risk) to develop the RNA viral infection, a therapeutically effective amount of a compound from Tables 2-8 or an analogue thereof, or a pharmaceutically acceptable salt of the compound from Tables 2-8 or the analogue thereof, thereby treating the RNA viral infection in the subject.

The method can, in some embodiments, comprise administrating one or more additional therapeutic agents to the subject. For example, the one or more additional therapeutic agents can comprise a binder of a receptor related to RNA viral infection. The binder of a receptor related to RNA viral infection can be a modulator for the receptor related to RNA viral infection, for example, an agonist or antagonist of the receptor related to RNA viral infection.

The RNA viral infection can be, for example, a coronavirus. In some embodiments, the RNA viral infection is SARS COV-1, SARS COV-2, the common cold, influenza, SARS, hepatitis C, hepatitis E, West Nile fever, Ebola virus disease, rabies, polio and measles, or a combination thereof. The International Committee on Taxonomy of Viruses (ICTV) classifies RNA viruses as those that belong to Group III, Group IV or Group V of the Baltimore classification system. Another term for RNA viruses is ribovirus. Viruses with RNA as their genetic material which also include DNA intermediates in their replication cycle are called retroviruses, and comprise Group VI of the Baltimore classification. Notable human retroviruses include HIV-1 and HIV-2, the cause of the disease AIDS. In some embodiments, the RNA viral infection is a results of viruses from Groups III, IV, V, or VI of the Baltimore classification system.

Non-limiting examples of RNA viral infection include Paramyxoviruses, Hendra and Nipah viruses, Measles, Severe acute respiratory syndrome coronavirus (SARS), COVID-19, Middle east respiratory syndrome coronavirus (MERS), Picornaviruses, Poliomyelitis (‘Polio’), Hepatitis A virus (HAV), Rotavirus, Human immunodeficiency virus (HIV), Human T-cell lymphotropic virus (HTLV), Hepatitis C virus (HCV), Hepatitis E virus (HEV), Rabies, Ebola virus disease (EVD), Marburg virus, Lassa fever, Lymphocytic choriomeningitis virus (LCMV), Arboviruses (‘ARthropod-BOrne viruses’), Japanese encephalitis (JE), West Nile fever, Yellow fever, Dengue fever, Zika virus, Equine encephalitis viruses, Chikungunya, O'nyong-nyong, Bunyaviruses, Rift valley fever and Crimean-Congo haemorrhagic fever, Hantavirus, or a combination thereof. The RNA viral infection can also be a complication due to a bacterial or parasitic infection.

A compound from Tables 2-8 or the analogue thereof can be administered in the form of a pro-drug. The compound from Tables 2-8 or the analogue thereof can be, for example, administered orally. In some embodiments, the compound from Tables 2-8 or the analogue thereof is administered in the form of a pill, a tablet, a microtablet, a pellet, a micropellet, a capsule, a capsule containing microtablets, or a liquid formulation. In some embodiments, the compound from Tables 2-8 or the analogue thereof is administered in the form of a capsule containing enteric coated microtablets.

The compound from Tables 2-8 or the analogue thereof or a pharmaceutically acceptable salt thereof can be administered in various frequency, for example, once, twice, or three times a day. In some embodiments, the compound of from Tables 2-8 or the analogue thereof or a pharmaceutically acceptable salt thereof can be administered no more than once, twice, or three times a day. In some embodiments, the compound from Tables 2-8 or the analogue thereof or a pharmaceutically acceptable salt thereof can be administered at least once, twice, or three times a day. In some embodiments, the compound from Tables 2-8 or the analogue thereof or a pharmaceutically acceptable salt thereof is administered once every day, every two days, every three days, every four days, or every five days. The duration for the treatment can vary. For example, the compound from Tables 2-8 or the analogue thereof or a pharmaceutically acceptable salt thereof can be administered over the course of at least one week, at least two weeks, at least three weeks, at least four weeks, at least five weeks, at least ten weeks, at least twenty weeks, at least twenty-six weeks, at least a year, or longer. In some embodiments, the compound from Tables 2-8 or the analogue thereof or a pharmaceutically acceptable salt thereof can be administered over the course of no more than five weeks, no more than ten weeks, no more than twenty weeks, no more than twenty-six weeks, or no more than a year.

Disclosed herein include methods for preventing, delaying the onset of, or treating an infection or a disease caused by a RNA virus. In some embodiments, the method comprises: administering to a subject in need thereof a composition comprising a compound or a pharmaceutically acceptable salt, ester, solvate, stereoisomer, tautomer, or prodrug thereof, thereby preventing, delaying the onset of, or treating the infection or the disease, wherein the compound is selected from the compounds listed in Tables 2-8.

Disclosed herein include methods for preventing, delaying the onset of, or treating an inflammatory effect of an infection or a disease caused by a RNA virus. In some embodiments, the method comprises: administering to a subject in need thereof a composition comprising a compound or a pharmaceutically acceptable salt, ester, solvate, stereoisomer, tautomer, or prodrug thereof, thereby preventing, delaying the onset of, or treating the inflammatory effect, wherein the compound is selected from the compounds listed in Tables 2-8.

Disclosed herein include methods for imparting resistance to an RNA virus to a cell. In some embodiments, the method comprises: contacting the cell with a composition comprising a compound or a pharmaceutically acceptable salt, ester, solvate, stereoisomer, tautomer, or prodrug thereof, thereby imparting resistance to the RNA virus to the cell, wherein the compound is selected from the compounds listed in Tables 2-8. The method can comprise: contacting a plurality of cells with the composition. The method can comprise: determining the infection rate of the plurality of cells after being contacted with the composition. In some embodiments, the cell expresses angiotensin-converting enzyme 2 (ACE2). In some embodiments, the cell is a lung cell, an enterocyte, an endothelial cell, an epithelial cell, a kidney cell, an arterial smooth muscle cell, a cell of the respiratory tract, or any combination thereof. In some embodiments, the cell is the cell of a subject. In some embodiments, the cell is in a subject. In some embodiments, contacting the cell with the composition is in a subject. In some embodiments, contacting the cell with the composition occurs in vitro, ex vivo, and/or in vivo.

In some embodiments, the inflammatory effect comprises respiratory failure, a sequela of respiratory failure, acute lung injury, or acute respiratory distress syndrome. In some embodiments, the sequela of respiratory failure comprises multi-organ failure. In some embodiments, the composition comprises a therapeutically or prophylactically effective amount of the compound. In some embodiments, the composition is a pharmaceutical composition comprising the compound and one or more pharmaceutically acceptable excipients.

The method can comprise: administering to the subject one or more additional antiviral agents. In some embodiments, at least one of the one or more additional antiviral agents is co-administered to the subject with the composition. In some embodiments, at least one of the one or more additional antiviral agents is administered to the subject before the administration of the composition, after the administration of the composition, or both. In some embodiments, the composition comprises one or more additional therapeutic agents. In some embodiments, the one or more additional therapeutic agents comprise one or more antiviral agents. In some embodiments, the antiviral agent is selected from the group consisting of a nucleoside or a non-nucleoside analogue reverse-transcriptase inhibitor, a nucleotide analogue reverse-transcriptase inhibitor, a NS3/4A serine protease inhibitor, a NS5B polymerase inhibitor, and interferon alpha.

In some embodiments, the composition is administered to the subject by intravenous administration, nasal administration, pulmonary administration, oral administration, parenteral administration, or nebulization. In some embodiments, the composition is aspirated into at least one lung of the subject. In some embodiments, the composition is in the form of powder, pill, tablet, microtablet, pellet, micropellet, capsule, capsule containing microtablets, liquid, aerosols, or nanoparticles. In some embodiments, the composition is in a formulation for administration to the lungs. In some embodiments, the composition is administered to the subject once, twice, or three times a day. In some embodiments, the composition is administered to the subject once every day, every two days, or every three days. In some embodiments, the composition is administered to the subject over the course of at least two weeks, at least three weeks, at least four weeks, or at least five weeks.

The method can comprise: measuring the viral titer of the RNA virus in the subject before administering the composition to the subject, after administering the composition to the subject, or both. In some embodiments, the viral titer is lung bulk virus titer. In some embodiments, administrating the composition results in reduction of the viral titer of the RNA virus in the subject as compared to that in the subject before administration of the composition. The method can comprise: determining global virus distribution in the lungs of the subject. The method can comprise: measuring a neutrophil density within the lungs of the subject. In some embodiments, administering the composition results in reduction of the neutrophil density within the lungs of the subject as compared to that in the subject before administration of the composition. The method can comprise: measuring a total necrotized cell count within the lungs of the subject. In some embodiments, administering the composition results in reduction of the total necrotized cell count in the subject as compared to that in the subject before administration of the composition. The method can comprise: measuring a total protein level within the lungs of the subject. In some embodiments, administering the composition results in reduction of the total protein level within the lungs of the subject as compared to that in the subject before administration of the composition.

Disclosed herein include methods for preventing, delaying the onset of, or treating an infection or a disease caused by a RNA virus. In some embodiments, the method comprises: administering to a subject in need thereof (1) a first compound selected from the compounds listed in Tables 2-8, or a pharmaceutically acceptable salt, ester, solvate, stereoisomer, tautomer, or prodrug thereof, and (2) a second compound selected from the compounds listed in Tables 2-8, or a pharmaceutically acceptable salt, ester, solvate, stereoisomer, tautomer, or prodrug thereof, thereby preventing, delaying the onset of, or treating the inflammatory effect, wherein the first compound and the second compound are different.

Disclosed herein include methods for preventing, delaying the onset of, or treating an inflammatory effect of an infection or a disease caused by a RNA virus. In some embodiments, the method comprises: administering to a subject in need thereof (1) a first compound selected from the compounds listed in Tables 2-8, or a pharmaceutically acceptable salt, ester, solvate, stereoisomer, tautomer, or prodrug thereof, and (2) a second compound selected from the compounds listed in Tables 2-8, or a pharmaceutically acceptable salt, ester, solvate, stereoisomer, tautomer, or prodrug thereof, thereby preventing, delaying the onset of, or treating the inflammatory effect, wherein the first compound and the second compound are different.

Disclosed herein include methods for imparting resistance to an RNA virus to a cell. In some embodiments, the method comprises: contacting the cell with (1) a first compound selected from the compounds listed in Tables 2-8, or a pharmaceutically acceptable salt, ester, solvate, stereoisomer, tautomer, or prodrug thereof, and (2) a second compound selected from the compounds listed in Tables 2-8, or a pharmaceutically acceptable salt, ester, solvate, stereoisomer, tautomer, or prodrug thereof, thereby imparting resistance to the RNA virus to the cell, wherein the first compound and the second compound are different. The method can comprise: contacting a plurality of cells with the first compound and the second compound. The method can comprise: determining the infection rate of the plurality of cells after being contacted with the first compound and the second compound. In some embodiments, the cell expresses angiotensin-converting enzyme 2 (ACE2). In some embodiments, the cell is a lung cell, an enterocyte, an endothelial cell, an epithelial cell, a kidney cell, an arterial smooth muscle cell, a cell of the respiratory tract, or any combination thereof. In some embodiments, the cell is the cell of a subject. In some embodiments, the cell is in a subject. In some embodiments, contacting the cell with the first compound and the second compound is in a subject. In some embodiments, contacting the cell with the first compound and the second compound occurs in vitro, ex vivo, and/or in vivo.

In some embodiments, the inflammatory effect comprises respiratory failure, a sequela of respiratory failure, acute lung injury, or acute respiratory distress syndrome, optionally the sequela of respiratory failure comprises multi-organ failure. The method can comprise: administering to the subject (3) a third compound selected from the compounds listed in Tables 2-8, or a pharmaceutically acceptable salt, ester, solvate, stereoisomer, tautomer, or prodrug thereof, wherein the first, second and third compounds are different.

In some embodiments, the first compound, the second compound, and/or the third compound is administered in a therapeutically or prophylactically effective amount. In some embodiments, the first, second and/or third compound is in a pharmaceutical composition comprising one or more pharmaceutically acceptable excipients. The method can comprise: administering to the subject one or more additional therapeutic agents. In some embodiments, the therapeutic agent is selected from the group consisting of a nucleoside or a non-nucleoside analogue reverse-transcriptase inhibitor, a nucleotide analogue reverse-transcriptase inhibitor, a NS3/4A serine protease inhibitor, a NS5B polymerase inhibitor, and interferon alpha. In some embodiments, at least one of the one or more additional therapeutic agents is administered to the subject before the administration of the first, second or third compound; after the administration of the first, second or third compound; or both.

In some embodiments, at least two of the first, second and third compounds are co-administered in a single composition or in separate compositions to the subject. In some embodiments, the first, second and third compounds are co-administered in a single composition or in separate compositions to the subject. In some embodiments, the first, second and/or third compound is administered to the subject by intravenous administration, nasal administration, pulmonary administration, oral administration, parenteral administration, nebulization, or a combination thereof. In some embodiments, the first, second and/or third compound is aspirated into at least one lung of the subject. In some embodiments, at least one of the first, second and third compounds is in a composition in the form of powder, pill, tablet, microtablet, pellet, micropellet, capsule, capsule containing microtablets, liquid, aerosols, or nanoparticles. In some embodiments, at least one of the first, second and third compounds is in a composition in a formulation for administration to the lungs. In some embodiments, at least one of the first, second and third compounds is administered to the subject once, twice, or three times a day. In some embodiments, at least one of the first, second and third compounds is administered to the subject once every day, every two days, or every three days. In some embodiments, at least one of the first, second and third compounds is administered to the subject over the course of at least two weeks, at least three weeks, at least four weeks, or at least five weeks.

The method can comprise: measuring the viral titer of the RNA virus in the subject before administering the first, second and/or the third compound to the subject, after administering the first, second and/or the third compound to the subject, or both, optionally the viral titer is lung bulk virus titer. In some embodiments, administrating the first, second and/or the third compound results in reduction of the viral titer of the RNA virus in the subject as compared to that in the subject before administration of the first, second and/or the third compound. The method can comprise: determining global virus distribution in the lungs of the subject. The method can comprise: measuring a neutrophil density within the lungs of the subject. In some embodiments, administering the first, second and/or the third compound results in reduction of the neutrophil density within the lungs of the subject as compared to that in the subject before administration of the first, second and/or the third compound. The method can comprise: measuring a total necrotized cell count within the lungs of the subject, optionally administering the first, second and/or the third compound results in reduction of the total necrotized cell count in the subject as compared to that in the subject before administration of the first, second and/or the third compound. The method can comprise: measuring a total protein level within the lungs of the subject. In some embodiments, administering the first, second and/or the third compound results in reduction of the total protein level within the lungs of the subject as compared to that in the subject before administration of the first, second and/or the third compound.

In some embodiments, the subject in need thereof is a subject that is suffering from the infection or the disease, or a subject that is at a risk for the infection or the disease. In some embodiments, the infection or the disease is in the respiratory tract of the subject. In some embodiments, the subject has been exposed to the RNA virus, is suspected to have been exposed to the RNA virus, or is at a risk of being exposed to the RNA virus. In some embodiments, the subject is a mammal (e.g., a human). In some embodiments, the RNA virus is a double-stranded RNA virus. In some embodiments, the RNA virus is a positive-sense single-stranded ssRNA virus. In some embodiments, the positive-sense single-stranded ssRNA virus is a coronavirus. In some embodiments, the coronavirus is an alpha coronavirus, a beta coronavirus, a gamma coronavirus, or a delta coronavirus. The coronavirus can be Middle East respiratory coronavirus (MERS-CoV), severe acute respiratory syndrome coronavirus (SARS-CoV), or SARS-CoV-2. In some embodiments, the disease is common cold, influenza, SARS, coronaviruses, COVID-19, hepatitis C, hepatitis E, West Nile fever, Ebola virus disease, rabies, polio, or measles.

Disclosed herein includes a kit, comprising a compound from Tables 2-8 or the analogue thereof, or a pharmaceutically acceptable salt thereof, and a label indicating that the kit is for the treatment or amelioration of one or more symptoms of an RNA viral infection. The compound from Tables 2-8 or the pharmaceutically acceptable salt thereof can be, for example, in a composition comprising the compound from Tables 2-8 or the analogue thereof, or the pharmaceutically acceptable salt thereof, and a pharmaceutically acceptable excipient. In some embodiments, the kit comprises: a first compound selected from the compounds listed in Tables 2-8, or a pharmaceutically acceptable salt, ester, solvate, stereoisomer, tautomer, or prodrug thereof; and a label indicating that the kit is for preventing, delaying the onset of, or treating an infection or a disease caused by a RNA virus. In some embodiments, the kit comprises: a first compound selected from the compounds listed in Tables 2-8, or a pharmaceutically acceptable salt, ester, solvate, stereoisomer, tautomer, or prodrug thereof; and a label indicating that the kit is for preventing, delaying the onset of, or treating an inflammatory effect of an infection or a disease caused by a RNA virus. In some embodiments, the kit comprises: a first compound selected from the compounds listed in Tables 2-8, or a pharmaceutically acceptable salt, ester, solvate, stereoisomer, tautomer, or prodrug thereof; and a label indicating that the kit is for imparting resistance to an RNA virus to a cell.

The kit can comprise: a second compound selected from the compounds listed in Tables 2-8, or a pharmaceutically acceptable salt, ester, solvate, stereoisomer, tautomer, or prodrug thereof, wherein the first compound and the second compound is different. The kit can comprise: a third compound selected from the compounds listed in Tables 2-8, or a pharmaceutically acceptable salt, ester, solvate, stereoisomer, tautomer, or prodrug thereof, wherein the first, second and third compound are different.

Also disclosed herein include a composition comprising a compound from Tables 2-8 or an analogue thereof, or a pharmaceutically acceptable salt of the compound from Tables 2-8 or an analogue thereof for use in the treatment of an RNA viral infection in a subject. In some embodiments, the treatment comprises administrating one or more additional therapeutic agents to the subject. The one or more additional therapeutic agents can, for example, comprise a binder of a receptor related to RNA viral infection.

Disclosed herein include compositions. In some embodiments, the composition comprises: a compound selected from the compounds listed in Tables 2-8, or a pharmaceutically acceptable salt, ester, solvate, stereoisomer, tautomer, or prodrug thereof, for use in preventing, delaying the onset of, or treating an infection or a disease caused by a RNA virus. In some embodiments, the composition comprises: a compound selected from the compounds listed in Tables 2-8, or a pharmaceutically acceptable salt, ester, solvate, stereoisomer, tautomer, or prodrug thereof, for use in preventing, delaying the onset of, or treating an inflammatory effect of an infection or a disease caused by a RNA virus. In some embodiments, the composition comprises: a compound selected from the compounds listed in Tables 2-8, or a pharmaceutically acceptable salt, ester, solvate, stereoisomer, tautomer, or prodrug thereof, for use in imparting resistance to an RNA virus to a cell.

### BRIEF DESCRIPTION OF THE DRAWINGS

FIG. 1 depicts a non-limiting exemplary validation funnel for in vitro and ex vivo validation of drug candidates with SARS-CoV-2 antiviral activity provided herein.

FIGS. 2A-2C depict data related to genome-wide loss-of-function genetic screening to identify the host factors involved in SARS-CoV-2 life cycle. FIG. 2A. Individual pooled-siRNAs were spotted into 384-well plates and reverse transfected into 3000 Caco-2 cells. 48 hours post seeding and transfection cells were infected with SARS-CoV-2 at an optimized MOI=0.625. At 48 hours post-infection, cells were fixed, immunolabeled with anti-SARS-CoV-2 nucleoprotein antibody and stained with DAPI. FIG. 2B. Example of images collected by high content imaging. The siRNAs transfected in each well are indicated. FIG. 2C. Average Z-score for all factors.

FIGS. 3A-3B depict data related to antiviral and cytotoxicity dose-response curves. 3000 HEK293T-ACE2 cells were added to 384-well plates pre-spotted with the SMAC-mimetics, Ciapavir (FIG. 3B) or AZD5582 (FIG. 3A), to bring the compounds to the indicated concentrations. 48 hours post seeding cells were infected with SARS-CoV-2 at an optimized MOI=0.625. At 48 hours post-infection, cells were fixed, immunolabeled with anti-SARS-CoV-2 nucleoprotein antibody and stained with DAPI. Relative infection (squares). Relative cell number (circles).

FIG. 4 provides a non-limiting exemplary compound screening workflow employed herein.

FIGS. 5A-5C depict cytotoxicity data. FIG. 5A-FIG. 5B depict the cytotoxicity of controls (DMSO, Apilimod DR, Remdesivir DR) and selected compounds when tested at 5 μM (FIG. 5A) and 1 μM (FIG. 5B). FIG. 5C depicts data related to cytotoxicity of the indicated compounds at 5 μM and 1 μM.

FIGS. 6A-6F depict infection data. FIGS. 6A-6D depict data related to the relative infection (circles) and relative cell count (squares) of the Apilimod (FIG. 6A, FIG. 6C) and Remdesivir (FIG. 6B, FIG. 6D) controls tested in the 1 μM screen (FIGS. 6A-6B) and the 5 μM screen (FIGS. 6C-6D) described below. FIGS. 6E-6H depict the effect of selected compounds on log₂FC SARS-CoV-2 infection (FIG. 6E and FIG. 6G) and normalized cell number (FIG. 6F and FIG. 6H) at 1 μM (FIGS. 6E-6F) and at 5 μM (FIGS. 6G-6H). FIG. 6I depicts correlation analysis of the effect of the selected compounds on log₂FC SARS-CoV-2 infection at 1 μM and at 5 μM. FIGS. 6J-6K depict correlation analysis of the effect of the selected compounds on normalized cell number and log₂FC SARS-CoV-2 infection at 1 μM (FIG. 6J) and at 5 μM (FIG. 6K).

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include methods for preventing, delaying the onset of, or treating an infection or a disease caused by a RNA virus. In some embodiments, the method comprises: administering to a subject in need thereof a composition comprising a compound or a pharmaceutically acceptable salt, ester, solvate, stereoisomer, tautomer, or prodrug thereof, thereby preventing, delaying the onset of, or treating the infection or the disease, wherein the compound is selected from the compounds listed in Tables 2-8.

Disclosed herein include methods for preventing, delaying the onset of, or treating an inflammatory effect of an infection or a disease caused by a RNA virus. In some embodiments, the method comprises: administering to a subject in need thereof a composition comprising a compound or a pharmaceutically acceptable salt, ester, solvate, stereoisomer, tautomer, or prodrug thereof, thereby preventing, delaying the onset of, or treating the inflammatory effect, wherein the compound is selected from the compounds listed in Tables 2-8.

Disclosed herein include methods for imparting resistance to an RNA virus to a cell. In some embodiments, the method comprises: contacting the cell with a composition comprising a compound or a pharmaceutically acceptable salt, ester, solvate, stereoisomer, tautomer, or prodrug thereof, thereby imparting resistance to the RNA virus to the cell, wherein the compound is selected from the compounds listed in Tables 2-8.

Disclosed herein include methods for preventing, delaying the onset of, or treating an infection or a disease caused by a RNA virus. In some embodiments, the method comprises: administering to a subject in need thereof (1) a first compound selected from the compounds listed in Tables 2-8, or a pharmaceutically acceptable salt, ester, solvate, stereoisomer, tautomer, or prodrug thereof, and (2) a second compound selected from the compounds listed in Tables 2-8, or a pharmaceutically acceptable salt, ester, solvate, stereoisomer, tautomer, or prodrug thereof, thereby preventing, delaying the onset of, or treating the inflammatory effect, wherein the first compound and the second compound are different.

Disclosed herein include methods for preventing, delaying the onset of, or treating an inflammatory effect of an infection or a disease caused by a RNA virus. In some embodiments, the method comprises: administering to a subject in need thereof (1) a first compound selected from the compounds listed in Tables 2-8, or a pharmaceutically acceptable salt, ester, solvate, stereoisomer, tautomer, or prodrug thereof, and (2) a second compound selected from the compounds listed in Tables 2-8, or a pharmaceutically acceptable salt, ester, solvate, stereoisomer, tautomer, or prodrug thereof, thereby preventing, delaying the onset of, or treating the inflammatory effect, wherein the first compound and the second compound are different.

Disclosed herein include methods for imparting resistance to an RNA virus to a cell. In some embodiments, the method comprises: contacting the cell with (1) a first compound selected from the compounds listed in Tables 2-8, or a pharmaceutically acceptable salt, ester, solvate, stereoisomer, tautomer, or prodrug thereof, and (2) a second compound selected from the compounds listed in Tables 2-8, or a pharmaceutically acceptable salt, ester, solvate, stereoisomer, tautomer, or prodrug thereof, thereby imparting resistance to the RNA virus to the cell, wherein the first compound and the second compound are different.

Disclosed herein include kits. In some embodiments, the kit comprises: a first compound selected from the compounds listed in Tables 2-8, or a pharmaceutically acceptable salt, ester, solvate, stereoisomer, tautomer, or prodrug thereof; and a label indicating that the kit is for preventing, delaying the onset of, or treating an infection or a disease caused by a RNA virus.

Disclosed herein include kits. In some embodiments, the kit comprises: a first compound selected from the compounds listed in Tables 2-8, or a pharmaceutically acceptable salt, ester, solvate, stereoisomer, tautomer, or prodrug thereof; and a label indicating that the kit is for preventing, delaying the onset of, or treating an inflammatory effect of an infection or a disease caused by a RNA virus.

Disclosed herein include kits. In some embodiments, the kit comprises: a first compound selected from the compounds listed in Tables 2-8, or a pharmaceutically acceptable salt, ester, solvate, stereoisomer, tautomer, or prodrug thereof; and a label indicating that the kit is for imparting resistance to an RNA virus to a cell.

Disclosed herein include compositions. In some embodiments, the composition comprises: a compound selected from the compounds listed in Tables 2-8, or a pharmaceutically acceptable salt, ester, solvate, stereoisomer, tautomer, or prodrug thereof, for use in preventing, delaying the onset of, or treating an infection or a disease caused by a RNA virus.

Disclosed herein include compositions. In some embodiments, the composition comprises: a compound selected from the compounds listed in Tables 2-8, or a pharmaceutically acceptable salt, ester, solvate, stereoisomer, tautomer, or prodrug thereof, for use in preventing, delaying the onset of, or treating an inflammatory effect of an infection or a disease caused by a RNA virus.

Disclosed herein include compositions. In some embodiments, the composition comprises: a compound selected from the compounds listed in Tables 2-8, or a pharmaceutically acceptable salt, ester, solvate, stereoisomer, tautomer, or prodrug thereof, for use in imparting resistance to an RNA virus to a cell.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

A “host factor,” as used herein, shall be given its ordinary meaning, and shall also refer to a biomolecule of a host wherein the biomolecule contributes to or inhibits SARS-CoV-2 replication.

As used herein, the term “loss-of-function” shall be given its ordinary meaning, and shall also refer to a reduced level of expression of a gene (either in RNA or in protein) or reduced or absent protein production or to a decreased ability of a gene to perform its biological function, e.g. to bind to another protein such as a receptor, to bind to DNA in one cell when compared to the level in another cell, or in one condition when compared to another condition. As used herein, “loss-of-function”, shall be given its ordinary meaning, and shall also refer to a reduction in gene expression, protein production, or protein activity that is from about 0 to about 100%, or from about 0 to about 75%, or from about 0% to about 50%, or from about 0% to about 40%, or from about 0% to about 30%, or from about 0% to about 20%, or from 0% to about 10%, or less than about 5% of that observed in a cell exhibiting normal or wild-type gene expression, protein production, or protein activity.

As used herein, the term “gain-of-function” shall be given its ordinary meaning, and shall also refer to an increase in the level of expression of a gene (either in RNA or in protein) or to an enhanced ability to perform its direct or indirect biological function, e.g. to bind to another protein such as a receptor, to bind to DNA in one cell when compared to the level in another cell, or in one condition when compared to another condition. As used herein, “gain-of-function” shall be given its ordinary meaning, and shall also refer to an increase in gene expression, protein production, or protein activity in a cell that is from about 0 to about 100%, or from about 0 to about 75%, or from about 0% to about 50%, or from about 0% to about 40%, or from about 0% to about 30%, or from about 0% to about 20%, or from 0% to about 10%, or less than about 5% higher than that in a cell exhibiting normal or wild-type gene expression, protein production, or protein activity.

As used herein, the term “expression” shall be given its ordinary meaning, and shall also refer to the cellular processes by which an RNA is produced by RNA polymerase (RNA expression) or a polypeptide is produced from RNA (protein expression).

As used herein, the term “short, interfering RNA” or “siRNA” shall be given its ordinary meaning, and shall also refer to an RNAi agent comprising an RNA duplex (referred to herein as a “duplex region”) that is approximately 19 base pairs (bp) in length and optionally further comprises one to three single-stranded overhangs. In some embodiments, an RNAi agent comprises a duplex region ranging from 15 bp to 29 bp in length and optionally further comprising one or two single-stranded overhangs. An siRNA may be formed from two RNA molecules that hybridize together, or may alternatively be generated from a single RNA molecule that includes a self-hybridizing portion. In general, free 5′-ends of siRNA molecules have phosphate groups, and free 3′-ends have hydroxyl groups. The duplex portion of an siRNA may, but typically does not, comprise one or more bulges consisting of one or more unpaired nucleotides. One strand of an siRNA includes a portion that hybridizes with a target transcript. In certain embodiments, one strand of the siRNA is precisely complementary with a region of the target transcript, meaning that the siRNA hybridizes to the target transcript without a single mismatch. In some embodiments, one or more mismatches between the siRNA and the targeted portion of the target transcript may exist. In some embodiments in which perfect complementarity is not achieved, any mismatches are generally located at or near the siRNA termini. In some embodiments, siRNAs mediate inhibition of gene expression by causing degradation of target transcripts.

“Individual” as used herein refers to a; person, human adult or child, mammal, or non-human primate.

“IC₅₀” as used herein, shall be given its ordinary meaning, and shall also refer to the molar concentration of a compound, for example a compound provided herein or an analogue thereof, which reduced infectivity by 50% in vitro.

“CC₅₀” as used herein shall be given its ordinary meaning, and shall also refer to the molar concentration of a compound, for example a compound provided herein or an analogue thereof, which reduced cell viability by 50% in vitro.

“Selectivity index or “SI” as used herein shall be given its ordinary meaning, and shall also refer to the relative cytotoxicity of a compound provided herein or an analogue thereof to the relative infectivity of said compound expressed as the ratio [CC₅₀ (cytotoxicity index)/IC₅₀ (infectivity index)].

“Antiviral” as used herein shall be given its ordinary meaning, and shall also refer to the reduction of viral activity, infectivity, replication, or any combination thereof, by a host factor disclosed herein, or a compound provided herein or an analogue thereof capable of modulating said host factor.

“Proviral” as used herein shall be given its ordinary meaning, and shall also refer to contribution to the viral activity, infectivity, replication, or any combination thereof, by a host factor disclosed herein, or a compound provided herein or an analogue thereof capable of modulating said host factor.

“Patient” as used herein refers to a mammal, e.g., a human, mouse, rat, guinea pig, dog, cat, horse, cow, pig, or non-human primate, such as a monkey, chimpanzee, baboon or rhesus.

“Pharmaceutically acceptable” as used herein means approved by a regulatory agency of the Federal or a state government or listed in the U.S. Pharmacopeia or other generally recognized pharmacopeia for use in animals, and more particularly in humans.

“Pharmaceutically acceptable salt” refers to a salt of a compound that is pharmaceutically acceptable and that possesses the desired pharmacological activity of the parent compound. Such salts include but are not limited to: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like.

“Pharmaceutically acceptable vehicle” refers to a diluent, adjuvant, excipient or carrier with which a compound disclosed herein is administered.

“Preventing” or “prevention” refers to a reduction in risk of acquiring a disease or disorder (i.e., causing at least one of the clinical symptoms of the disease not to develop in a patient that may be exposed to or predisposed to the disease but does not yet experience or display symptoms of the disease).

“Prodrug” refers to a derivative of a drug molecule that requires a transformation within the body to release the active drug. Prodrugs are frequently (though not necessarily) pharmacologically inactive until converted to the parent drug. Typically, prodrugs are designed to overcome pharmaceutical and/or pharmacokinetically based problems associated with the parent drug molecule that would otherwise limit the clinical usefulness of the drug.

“Promoiety” refers to a form of protecting group that when used to mask a functional group within a drug molecule converts the drug into a prodrug. Typically, the promoiety will be attached to the drug via bond(s) that are cleaved by enzymatic or non-enzymatic means in vivo. Ideally, the promoiety is rapidly cleared from the body upon cleavage from the prodrug.

“Protecting group” refers to a grouping of atoms that when attached to a reactive group in a molecule masks, reduces or prevents that reactivity. Examples of protecting groups can be found in Green et al., “Protective Groups in Organic Chemistry”, (Wiley, 2.sup.nd ed. 1991) and Harrison et al., “Compendium of Synthetic Organic Methods”, Vols. 1 8 (John Wiley and Sons, 1971 1996). Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“SES”), trityl and substituted trityl groups, allyloxycarbonyl, 9-fluorenylmethyloxycarbonyl (“FMOC”), nitro-veratryloxycarbonyl (“NVOC”) and the like. Representative hydroxy protecting groups include, but are not limited to, those where the hydroxy group is either acylated or alkylated such as benzyl, and trityl ethers as well as alkyl ethers, tetrahydropyranyl ethers, trialkylsilyl ethers and allyl ethers.

“Treating” or “treatment” of any disease or disorder as used herein, refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the patient. In yet another embodiment, “treating” or “treatment” refers to inhibiting the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

“Therapeutically effective amount” as used herein, means the amount of a compound that, when administered to an individual for treating a disease, is sufficient to effect such treatment for the disease or to achieve the desired clinical response. The “therapeutically effective amount” will vary depending on the compound, the disease and its severity and the age, weight, etc., of the patient to be treated.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice; rats; rabbits; guinea pigs; dogs; cats; sheep; goats; cows; horses; primates, such as monkeys, chimpanzees, and apes, and, in particular, humans.

As used herein, a “patient” refers to a subject that is being treated by a medical professional, such as a Medical Doctor (i.e. Doctor of Allopathic medicine or Doctor of Osteopathic medicine) or a Doctor of Veterinary Medicine, to attempt to cure, or at least ameliorate the effects of, a particular disease or disorder or to prevent the disease or disorder from occurring in the first place.

As used herein, “administration” or “administering” refers to a method of giving a dosage of a pharmaceutically active ingredient to a vertebrate.

As used herein, a “dosage” refers to an amount of therapeutic agent administered to a patient.

As used herein, a “daily dosage” refers to the total amount of therapeutic agent administered to a patient in a day.

As used herein, the term “therapeutic agent” means a substance that is effective in the treatment of a disease or condition.

As used herein, “therapeutically effective amount” or “pharmaceutically effective amount” is meant an amount of therapeutic agent, which has a therapeutic effect. The dosages of a pharmaceutically active ingredient which are useful in treatment are therapeutically effective amounts. Thus, as used herein, a therapeutically effective amount means those amounts of therapeutic agent which produce the desired therapeutic effect as judged by clinical trial results and/or model animal studies.

As used herein, a “therapeutic effect” relieves, to some extent, one or more of the symptoms of a disease or disorder. For example, a therapeutic effect may be observed by a reduction of the subjective discomfort that is communicated by a subject (e.g., reduced discomfort noted in self-administered patient questionnaire). “Treat,” “treatment,” or “treating,” as used herein refers to administering a therapeutic agent or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition.

As used herein, EC₅₀ is the value of a graded dose response curve that represents the concentration of a compound where 50% of its maximal effect is observed.

As used herein, SI=CC₅₀/EC₅₀. The selectivity index (SI) is a ratio that measures the window between cytotoxicity and antiviral activity by dividing the CC₅₀ value into the EC₅₀ value. The higher the SI ratio, the theoretically more effective and safe a drug would be during in vivo treatment for a given viral infection.

“Ki” as used herein refers to the kinetic inhibition constant in molar concentration units which denotes the affinity of the compound for the receptor related to RNA viral infection as measured by a binding assay or as calculated from the IC₅₀ value using the Cheng-Prusoff equation.

### The Immediate Need for Drugs Targeting COVID-19 Requires a Drug Repurposing Strategy.

The scientific community has mobilized around the novel coronavirus, SARS-CoV-2, which causes the pneumonia like disease named COVID-19. Until the recent emergency use approval of Remdesivir, which provides modest improvement of outcomes, there were no FDA approved drugs or vaccines for SARS-CoV-2 nor any of the other coronaviruses (CoVs). However, the more recent interim results published from the WHO's Solidarity Trial show Remdesivir had little or no effect on overall mortality, initiation of ventilation, and duration of hospital stay in hospitalized patients. So far, only corticosteroids, such as dexamethasone have shown any efficacy against severe and critical COVID-19. Thus, the development of effective antivirals to limit COVID-19 replication represents an urgent public health need.

De novo development of antiviral therapies usually requires 10-17 years at a cost of over $2 billion. Thus, repositioning clinically evaluated drugs represents one of the most practicable strategies for the rapid identification and deployment of treatments for emerging infectious diseases such as COVID-19. Toward this end, the repurposing of several approved antiviral therapies has been the focus of clinical investigations, including the HIV-1 protease inhibitors lopinavir/ritonavir, the hepatitis C virus protease inhibitor danoprevir, and the influenza antiviral favipiravir (T-705, Avigan). Each of these potential repurposed drugs, and also Remdesivir, a viral RNA polymerase inhibitor, represent direct acting antivirals that target viral proteins. Yet, the majority of drugs that are FDA approved or within clinical development target human proteins. For example, within the ReFRAME library, the most comprehensive open-access repurposing library created to date, just 14% of compounds are for infectious disease indications. The remaining compounds target human host proteins. Thus, an efficient drug repurposing strategy will require a comprehensive understanding of the host-pathogen interactions that regulate viral replication and disease.

### Novel Host Proteins as SARS-CoV-2 Therapeutic Targets.

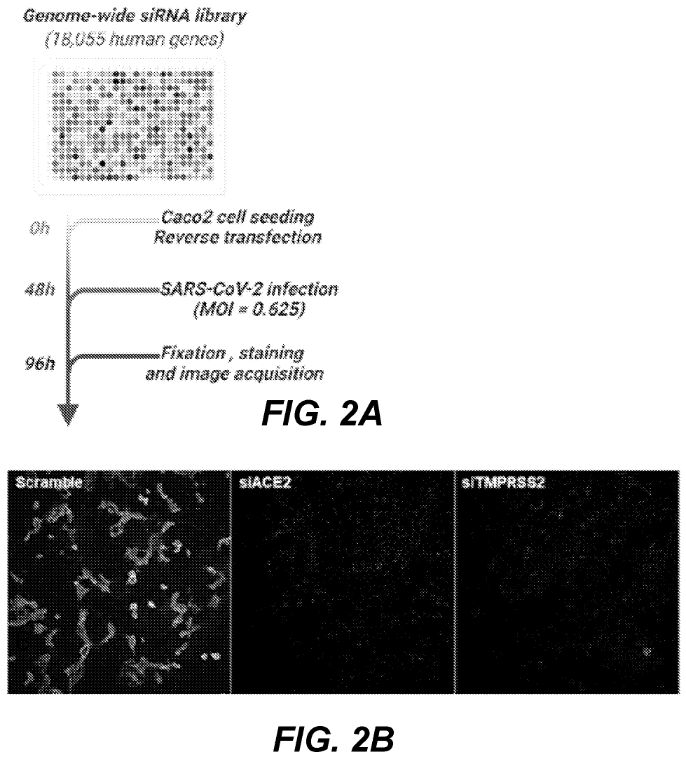

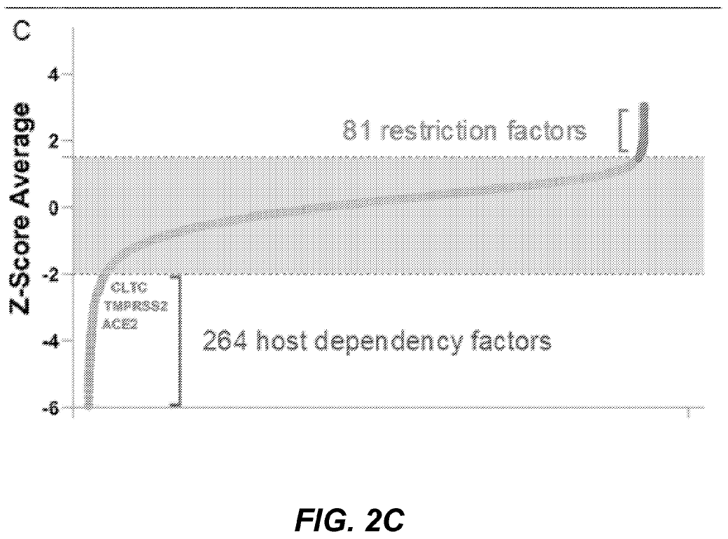

The complexity of the host-pathogen interactions that underlie outcome to viral exposure is reflected in the variability of clinical pathologies, with data suggesting ˜80% of infections are mild or asymptomatic, 15% are severe, requiring oxygen, and 5% are critical infections, requiring ventilation. The pathogenic outcome of a virus infection, while complex, depends on the ability of the virus to replicate, as well as the ability of the host to mount an immune response, amongst other factors. Critical host factors that impact on SARS-CoV-2 replication include the angiotensin-converting enzyme 2 (ACE2), which has been identified as a cellular receptor for the virus, and the serine protease TMPRSS2, which primes the SARS-CoV-2 S protein for viral entry into cells. In fact, the clinically proven serine protease inhibitor camostat mesylate, which is active against TMPRSS2, displayed in vitro activity against SARS-CoV-2, thus proving that host factors can be perturbed by chemical inhibitors to modulate viral replication. However, the wealth of host factors that impact on SARS-CoV-2 replication have yet to be elucidated. To identify repurposed drugs that target host proteins that govern SARS-CoV-2 replication, genetic screens were leveraged for host factors that modulate viral replication (FIGS. 2A-C and 3A-B). These factors can be targeted with repurposed drugs both within in vitro cellular models and in vivo animal models of and represent potential therapeutic targets for COVID-19. There are several conceptually as well as technologically innovative aspects of the repurposing strategy as described herein.

Current approaches for antiviral development and repurposing mainly target virus-specific proteins. This strategy can limit potential toxicities since targets are usually not present in the host, offering a large therapeutic window. However, for drug repurposing this represents a significant disadvantage, as described above, the majority of drugs that have been FDA approved or that are in clinical development target host factors. Thus, the conceptually innovative approach disclosed herein to repurpose drugs as host-directed antivirals offers several advantages. 1) A larger pool of targets. RNA viruses such as SARS-CoV-2 express very few viral proteins that can be targeted by drugs. By targeting host proteins, the number of potential drug targets greatly increases. 2) Host targets generally present a higher genetic barrier to resistance than viral targets. Take for example, the three classes of drugs approved to treat influenza infections, which target M2, NA, and PA viral proteins (the last was FDA approved in 2018); resistance has developed to all three. 3) Since CoVs are likely to share common strategies for replication, multiple viruses will possibly share dependence on common host pathways, thus making it likely that drugs will have broad spectrum activity against multiple CoVs. In fact, most non-infectious disease drugs target host factors, even those that are not dysregulated or dysfunctional within disease. For a drug repurposing strategy, since most compounds in the library, by definition, possess reasonable safety profiles, and thus will likely harbor broad therapeutic windows, the risks associated with host-directed therapies are largely mitigated.

### Deep Learning to Identify Novel Repurposing Drugs for COVID-19 Therapeutics

By determining the level of similarity to existing drugs, activities of novel compounds can be predicted as described herein. Using this concept of mechanism-of-action profiling, existing or off-patent drugs can be repurposed for use against other disorders. To achieve this, data sources of experimentally determined protein-molecule binding pairs described herein are used to generate a highly curated proprietary database and transform 3-dimensional molecular compounds into descriptive machine-readable bitvector “fingerprints”. A proprietary digital chemistry-based method described herein is used to train predictive models in an automated and efficient manner. A probabilistic—rather than a deterministic—approach to chemical-similarity-based protein target prediction was developed to avoid bias towards particular chemical groups.

The field of deep learning using neural networks has advanced at breathtaking speed in the past several years, far outpacing the typical progress made in mathematics and data science. The approaches and applications used permeate virtually every industry. Implementations of drug repurposing sometimes rely on chemomimetics—the principle that similar compounds have similar properties, such as the same protein targets. Similarity between elements—for instance, chemical composition of drug molecules—implies correlation at therapeutic effect level, or at drug target level. Thus, a large database of binding pairs can provide support about the mechanisms of action of bioactive compounds and predict or explain the toxicity of abandoned drugs. Such support are is on the transitivity principle that a compound A which is chemically similar to another compound B has increased likelihood of binding the same proteins as compound B. Accordingly, if A is found to have a biological activity, its mechanism or toxicity might be due to binding the same proteins as compound B. Given this assumption, methods were implemented to determine similarity-predicted activity.

Ideal for machine-readability, chemical information is derived from the intrinsic properties of small molecules, including their atomic connectivity, biophysical attributes, and pharmacophores. Importantly, chemical structures can be readily converted to digital fingerprints for use in statistical modeling software. Unlike other approaches that require expensive equipment and experimental approaches to collect data required for model training, cheminformatic data can be computed algorithmically, and serves as the basis for all predictive algorithms. Binding data derived from previously collected 3rd party experiments is used as described herein, so no new experiments are needed for input training set. Predictions rely on proprietary 2D and 3D fingerprints. Thus, a large effort has been devoted to generating appropriate fingerprints—bitstring representations of a molecule, based on unique patterns. The molecule is abstracted into a string of 0s and 1s through a mathematical hashing protocol. As there are a number of representation algorithms available, many of the popular protocols from JChem Extended Connectivity Fingerprint ECFP, RDKit (Morgan, FeatMorgan, AtomPair, Torsion, RDKit, Avalon, Layered, MACCS, Pattern), and CDK (Standard, Extended, PubChem, MACCS, Estate) were analyzed extensively. The platform employs a unique blend of these and other fingerprints, as determined through correlation analysis and backwards feature elimination. Higher dimensional feature space has the ability to capture much more information about individual molecules. 3-Dimensional fingerprints in the discovery platform provided herein are generated using four different techniques, of increasing complexity and accuracy: 1) Shape-based steric surface features, 2) Electrostatic regions of positive and negative charge, 3) Pharmacophore features required for molecular recognition, including hydrophobicity regions and donor/acceptor points with directionality vectors, and 4) ‘Fields’ which are larger regions within and around the molecule that represent energetic potentials. Abstracting these methods into machine-readable bitstrings is laborious and extremely computationally intensive, but once accomplished, provides far greater predictive power than 2D methods alone. Resource bottlenecks are alleviated by running in a cloud-based parallel computing environment.

A vast dataset of viable molecules was assembled to be used as a test query set, including a digitization of all US-approved small molecules. This data was sourced from NCATS Pharmaceutical Collection (NPC), DrugBank, DrugCentral, and the FDA NDC Database, a universal product identifier for drugs, published by the FDA. The recording of these drug products assists in the drug repurposing process. (https://www.fda.gov/drugs/drug-approvals-and-databases/ndc-product-file-definitions). With the information fingerprinted and enriched as mentioned above, predictions using principles of Artificial Intelligence (AI) can be made.

Recently, progress has been made using chemical structures for drug repurposing. A variant of the artificial neural network (ANN) architecture called multilayered perceptron (MLP) was used as described herein. Multiple hidden layers were tested and optimized hyperparameters to train the model with the goal of achieving a predictive accuracy measured by Cohen's kappa of 70%, a benchmark accepted by the statistics community.

A probabilistic-based classification approach provided herein, which removes biases intrinsic to deterministic-based similarity algorithms, was employed. Similarity searching and Classifications are examples of “supervised learning”. These methods require a training set of known similar or “actives” to teach the model right from wrong. Aside from the necessary pre-labeled classifications of true positives as well as true negatives for input data, there are several other requirements when using supervised learning probabilistic classification methods, including the need for sufficiently large, balanced, and clean training dataset.

### Drug Repurposing Screen for SARS-CoV-2 Antivirals AND Deep Learning Converge on Novel Drug for the Treatment of COVID-19

A cell-based drug repurposing screen to identify compounds that inhibit SARS-CoV-2 replication has been reported. An initial screening assay was established that utilizes the fact that viral infection induces a cytopathic effect in mammalian cell culture, as well as orthogonal immunofluorescence based assays in various human cell lines and primary human cells to validate effects on viral replication (FIG. 2B). The novel ReFRAME (Repurposing, Focused Rescue, and Accelerated MedChem) drug repositioning collection was screened to identify compounds that prevent SARS-CoV-2 from causing a viral cytopathic effect upon infection of cells in culture, and over 30 clinical stage molecules that may function as SARS-CoV2 antivirals were validated in multiple orthogonal assays.

One of the drugs identified in the screen of the ReFRAME library was also predicted using the deep learning algorithms described herein. Pagoclone was identified in a prediction model for Papain-like protease (PLPro), a known SARS-CoV-2 target, from a list of existing repurposable drugs. Pagoclone is an anxiolytic agent from the cyclopyrrolone family, related to better-known drugs such as the sleeping medication zopiclone. Pagoclone belongs to the class of nonbenzodiazepines, which have similar effects to the older benzodiazepine group, but with different chemical structures. It binds with roughly equivalent high affinity (0.7-9.1 nM) to the benzodiazepine binding site of human GABAA receptors containing either an α1, α2, α3 or α5 subunit. It is a partial agonist at α1-, α2- and α5-containing GABAA receptors and a full agonist at receptors containing an α3 subunit. Pagoclone was last studied in Phase 2/3 clinical trials for stuttering and for premature ejaculation in men and has never been commercialized.

Workflow to Predict Repurposeable Drugs that Target Novel Host Factors that Impact on SARS-CoV-2 Replication

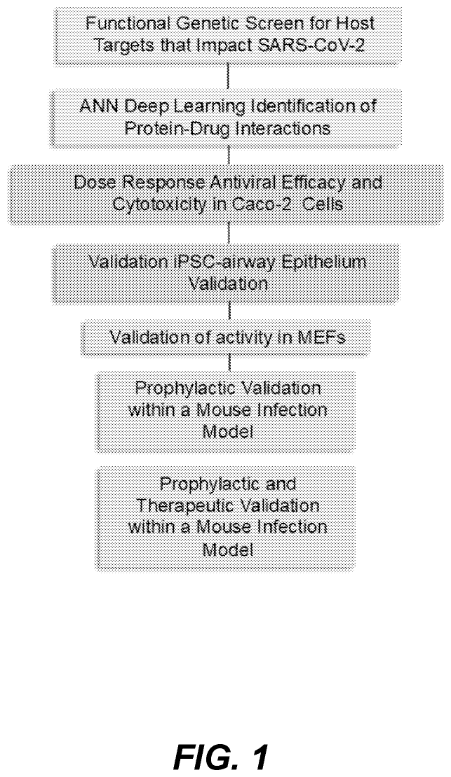

Given the validation of the algorithm described above, a workflow to identify additional repurposed drugs for novel host factor targets was designed (see Examples 1-12 below). This workflow, depicted in FIG. 1, started with a global genetic screen to identify host factors critical to the control of viral replication and immunological control (see Examples 1-2). After genetic screening, the ANN described herein used these data to identify host factors for which modeling could be conducted. Once appropriate targets had been identified the sophisticated ANN deep learning approach was used to predict repurposed drugs that would engage said target host factors (see Examples 3-5). Targets from siRNA screen with favorable characteristics for developing ligand-based predictive models are listed in Table 1 and discussed in detail below. Drugs were predicted to engage one or more of eight target proteins. A testing funnel can establish their efficacy in inhibiting SARS-CoV-2 replication first within in vitro and ex vivo cellular models of infection and then within both prophylactic and therapeutic tests within in vivo mouse models (see Examples 6-12). Experimental testing of compounds selected for analysis with the methods described herein identified 35 small molecules (see Tables 2-8) meeting the thresholds of infection log₂FC<2*Stdv of negative control DMSO, and normalized cell number>0.65 (Example 12).

TABLE 1
Targets from siRNA screen with favorable characteristics
for developing ligand-based predictive models

| GENE ID | UNIPROT ID | NAME |
|---|---|---|
| PFKFB3 | Q16875 (F263_HUMAN) | 6-phosphofructo-2-kinase/fructose-2,6- |
| | | bisphosphatase 3 |
| CCND3 | P30281 (CCND3_HUMAN) | G1/S- specific cyclin-D3 |
| KDM1A | O60341 (KDM1A_HUMAN) | LYSINE DEMETHYLASE 1A (human) |
| BIRC2 | Q13490 (BIRC2_HUMAN) | Baculoviral IAP repeat-containing protein 2 |
| DAGLA | Q9Y4D2 (DGLA_HUMAN) | Diacylglycerol lipase alpha (human) |
| CAPN1 | P07384 (CAN1_HUMAN) | Calpain-1 catalytic subunit |
| KDM5B | Q9UGL1 (KDM5B_HUMAN) | Lysine-specific demethylase 5B |
| MAPK11 | Q15759 (MK11_HUMAN) | Mitogen-activated protein kinase 11 |

Exemplary Targets from siRNA Screen with Favorable Characteristics for Developing Ligand-Based Predictive Models

### Type I. Antiviral Factors

The following are non-limiting examples of target host factors with antiviral activities and favorable characteristics for further development of ligand-based predictive models.

### Target 1. 6-phosphofructo-2-kinase fructose-2,6-bisphosphatase 3

Identification: UniProtKB—Q16875 (F263_HUMAN)

Gene: PFKFB3

Evidence: primary siRNA screen and validation screen described herein

Alternative Names/Synonyms

IPFK2, PFK2, iPFK-2, 6-phosphofructo-2-kinase/fructose-2,6-bisphosphatase 3, 6-phosphofructo-2-kinase/fructose-2,6-bisphosphatase, 6PF-2-K/Fru-2,6-P2ase 3, 6PF-2-K/Fru-2,6-P2ase brain/placenta-type isozyme, PFK/FBPase 3, fructose-6-phosphate, 2-kinase/fructose-2, 6-bisphosphatase, inducible 6-phosphofructo-2-kinase/fructose-2,6-bisphosphatase

Structure and Sequence

6-phosphofructo-2-kinase/fructose-2,6-bisphosphatase 3, also referred to as IPFK2, is a bifunctional protein encoded on chromosome 10 by the PFKFB3 gene. Alternative splicing of the gene produces 4 isoforms of a polypeptide made up of 520 amino acid residues. This enzyme is a homodimeric protein that has a N-terminal kinase domain and a C-terminal bisphosphatase domain (Manzano et al., 1998). The sequence of this protein can be viewed here, https://www.ncbi.nlm.nih.gov/protein/Q16875. A detailed review analysis of the available structures and binding pockets of PFKFB3 is described in Brooke et al., 2014.

Known Locations and Abundance

IPFK2 is expressed ubiquitously but mostly in fat followed by bone marrow, kidney, brain and the appendix (UniProt; NCBI Gene). PFKFB3 expression was analyzed and found to be most prominent in the muscle, kidney, lung and brain with less expression observed in the spleen, heart, liver and pancreas.

Physiology and Disease

6-phosphofructo-2-kinase/fructose-2,6-bisphosphatase 3, also known as PFK-2/FBPase 3 or PFKFB3, catalyzes the synthesis and the reverse hydrolytic degradation of fructose 2,6-bisphosphate. While PFKFB3 exhibits bifunctionality, its kinase activity is approximately 700 times more dominant than the phosphatase activity. The kinase reaction product is an important factor involved in the regulation of glycolysis. Fructose 2,6-bisphosphate is an allosteric activator for PFK1, a rate-limiting enzyme that catalyzes a rate-limiting step of glycolysis. PFKFB3 is also responsible for inducing “tumor endothelial cell vessel sprouting, branching and directional migration, promoting cell cycle progression at G1/S phases; and inhibiting apoptosis”.

Aerobic glycolysis is favored by cancer cells to produce large amounts of energy. Cancer cells' need for excessive energy through glycolysis is referred to as the “Warburg” effect. The crucial role that PFKFB3 has over the regulation of the glycolytic pathway has led scientists to evaluate the effects of PFKFB3 inhibition in cancerous cells. Abnormally high glycolytic activity in hypoxic tumors has been attributed to overexpression of PFKFB3. PFKFB3 and another isoform, PFKFB4, are overexpressed in a wide range of cancers including lung, breast, colon, pancreas, ovary, and thyroid tumors. PFKFB3 is a popular target for cancer therapy. Inhibition of this enzyme would decrease production of the allosteric activator, F2,6BP, for the rate-limiting enzyme of glycolysis, PFK1. This reduction in glycolytic activity would dwindle the preferred form of energy production for rapidly proliferating cancer cells.

PFKFB3 has an important role in antiviral innate immunity. Type I Interferon signaling is triggered by the detection of viral nucleic acids. This antiviral signaling induces a metabolic change, predominantly in macrophages, that aims to upregulate PFKFB3. Elevated PFKFB3 activity results in an increased production of fructose-2,6-bisphosphate, which can enhance glycolysis to support antiviral defense via increased metabolic energy Small molecule activators could serve a purpose to help fight off infections.

Target Relevance

In efforts to support innate immune defense, such as the engulfment and degradation of virus-infected cells by macrophages, important metabolic changes are induced by type I interferon signals. PFKFB3-driven glycolysis has been proven to benefit the antiviral capacity of macrophages in models infected with the RNA virus, Vesicular stomatitis. Without being bound by any particular theory, this same strategic metabolic change that acts to increase PFKFB3 and its enhancement of glycolysis could be occurring in organisms infected with SARS-CoV-2. The negative effect on cell viability in response to PFKFB3 LOF, demonstrates that PFKFB3 has antiviral properties, particularly in defense against SARS-CoV-2. Various compounds capable of targeting PFKFB4 were identified.

### Target 2. G1/S-Specific Cyclin-D3

Identification: UniProtKB—P30281 (CCND3_HUMAN)

Gene: CCND3

Evidence: primary siRNA screen and validation screen described herein

Alternative Names/Synonyms

G1/S-specific cyclin-D3, D3-type cyclin

Structure and Sequence

The G1/S-specific cyclin-D3 is encoded by the CCND3 gene located on chromosome 6. Alternative splicing of the CCND3 gene produces 4 isoforms. Cyclin D3 commonly forms a serine/threonine kinase holoenzyme complex with cyclin-dependent kinase 4 or 6 to carry out its function as a cell cycle regulator for the G₁ to S phase transition. The structure of the CDK4/cyclin 3 complex is elaborated on by Takaki et al., 2009. The detailed sequence of this protein is described here, https://www.ncbi.nlm.nih.gov/protein/P30281.

Known Locations and Abundance

Cyclin D3 is ubiquitously expressed in humans and is the most widely expressed of the D-type cyclins.

Physiology and Disease

G1/S-specific cyclin-D3 belongs to a family of cyclins and the subfamily of D-type cyclins, D1, D2 and D3, that have key roles in the G1-phase of the cell cycle for genome replication. D-type cyclins activate cyclin-dependent kinases 4 and 6 resulting in cell cycle progression from the G1 to S phase. Cyclin D3 acts as the regulatory subunit of CDK4 or CDK6 when in complex. Cyclin D3 also interacts with the retinoblastoma protein (Rb) which functions as a tumor suppressor and negative growth regulator. When Rb is underphosphorylated or in its unphosphorylated form, it inhibits the progression from the G₁ phase of the cell cycle. To lift this restriction, cyclin D3 can form a complex with and activate CDK4 and CDK6 to phosphorylate Rb protein thereby inactivating its function as a negative growth regulator and allowing the necessary gene expression for cell cycle progression. In addition to the role cyclin D3 has in cell cycle progression, it also interacts with the human activating transcription factor 5 (hATF5) to colocalize in the nucleus and potentiate its transcriptional activity.

Genetic mutations in the CCND3 gene is linked to splenic diffuse red pulp lymphoma, and the CCND3 gene serves as a genetic marker of tumor progression in bladder urothelial carcinoma. In non-small cell lung cancer tissues, cyclin D3 is abnormally regulated by overexpressed Hes3 suggesting a role in cancer cell proliferation and use of CCND3 as a cancer marker.

Cyclin D3 has been proven to have antiviral effects against the influenza A virus. It disrupts virion formation and weakens viral replication by competitively inhibiting a necessary protein-protein interaction between the M1-M2 viral proteins. The antiviral effect of Cyclin D3 is further supported by the evidence of a CCND3 KD experiment demonstrating increased viral progeny. The cyclin D3 protein has interactions with the HIV-1 tat protein and is upregulated in response to the HIV matrix protein p17. It has also been reported that cyclin D3 serves a purpose in the translocation of a trans activator protein of herpes simplex virus 1 (HSV-1), ICP0, and the stabilization and activation of G₁-phase cyclins. The gene encoding this subunit is downregulated by the HIV-1 protein Vpr.

Target Relevance

(1) Cyclin D3 directly interacts with the viral matrix protein 2 (M2) of the influenza A virus and consequently disrupts the necessary M1-M2 interaction for proper virion formation. Without being bound by any particular theory, cyclin D3 could potentially exhibit antiviral activity by competitively binding to viral proteins necessary for assembly of progeny virions in the novel coronavirus SARS-CoV-2; given it is also an RNA virus. The negative effect on cell viability in response to CCND3 LOF, demonstrates that CCND3 has antiviral properties.

(2) ORF7A, a viral protein of SARS-CoV, reduces cyclin D3 mRNA transcription and expression. The resultant cyclin D3 deficiency negatively affects the downstream cascade mediated by cyclin D3 that normally grants cell cycle progression into the S phase. Without sufficient levels of cyclin D3 due to ORF7A, cell growth is inhibited and G0/G1 phase arrest can be induced by ORF7A expression. Without being bound by any particular theory, this virus-induced early cell death has been suggested to favor coronavirus replication and pathogenicity. The novel coronavirus, SARS-CoV-2, genome also encodes ORF7a as an accessory protein. Without being bound by any particular theory, given the highly shared sequence identity and similarity of SARS-CoV and SARS-CoV-2 it is believed that the same negative regulatory effects of ORF7a in SARS-CoV are occurring against host cyclin D3 by the ORF7a of SARS-CoV-2. Compounds capable of targeting CCND3 that exhibit sufficient antiviral activity (log₂FC<2*Stdv of negative control DMSO) and sufficiently low cytotoxicity (normalized cell number>0.65) were identified in Example 12 and are listed in Table 2.

TABLE 2
Compounds Targeting CCND3

| Drug Name | Synonyms | CAS | Drugbank ID | Formula |
|---|---|---|---|---|
| ch-5132799, | PA-799 MEN-1611 | 1007207-67-1 | DB13051 | C15H19N7O3S |
| ch5132799 | | | | |
| cc-401, cc 401 | JNK-401 | 395104-30-0 | DB12432 | C22H24N6O |
| dihydrochloride | | | | |
| nemiralisib | GSK-2269557 | 1254036-71-9 | | C₂₆H₂₈N₆O |
| (GSK2269557) | | | | |
| pictilisib, gdc- 0941 | RG-7321 (tablet) GDC- | 957054-30-7 | DB11663 | C23H27N7O3S 2 |
| | 0941 | | | |
| | pictilisib (tablet) PI3 kinase | | | |
| | inhibs | | | |
| bimiralisib, pqr309 | NCB-5 PQR-309 | 1225037-39-7 | DB14846 | C17H20F3N7O 2 |
| | 6Z3QHB00LB | | | |
| lxs196 | IDE196 | 1874276-76-2 | | C₂₂H₂₃F₃N₈O |
| mk-8245 | | 1030612-90-8 | DB13024 | C17H16BrFN6 O4 |

### Target 3. Lysine Demethylase 1A (Human)

Identification: UniProtKB—O60341 (KDM1A_HUMAN)

Gene: KDM1A

Evidence: primary siRNA screen and validation screen described herein

Alternative Names/Synonyms

AOF2, BHC110, CPRF, KDM1, LSD1, lysine-specific histone demethylase 1A, BRAF35-HDAC complex protein BHC110, FAD-binding protein BRAF35-HDAC complex, 110 kDa subunit, amine oxidase (flavin containing) domain 2, flavin-containing amine oxidase domain-containing protein 2

Structure and Sequence

Lysine demethylase 1A, also referred to as LSD1, is a chromatin modifying enzyme that is encoded by the KDM1A gene located on chromosome 1. LSD1 is localized in the nucleus where it functions as a specific amine oxidase to demethylate substrates through an oxidative reaction. LSD1 has three domains, a N-terminal flexible region and C-terminal tail. The C-terminal of LSD1 contains an amine oxidase-like (AOL) domain which itself contains the FAD-binding and substrate-binding domains. The large catalytic cavity of LSD1 lies at the interface of these two subdomains. The SWIRM domain is located near the N-terminal and is believed to be important for the stability of LSD1. The third domain, Tower, is found between the AOL domain and acts as the binding site for CoREST thereby mediating the interaction between LSD1 and CoREST. In addition to being a component of CoREST, LSD1 also is a part of CtBP and HDAC corepressor complexes. The crystal structure of this oxidoreductase can be viewed here, https://www.rcsb.org/structure/2DW4 and described in detail here, https://pubmed.ncbi.nlm.nih.gov/18039463/by Mimasu et al., 2008.

Known Locations and Abundance

This protein is ubiquitously expressed but it is abundantly present in the testis (NCBI Gene).

Physiology and Disease

LSD1 acts to specifically demethylate the mono- and dimethylated lysine or lysine residues of histone H3 thereby modifying chromatin structure and consequently affecting gene transcription. For example, LSD1 acts as a transcriptional corepressor on K4 of histone H3 by demethylating the N-terminal tail of the histone. Contrastingly, LSD1 can also act as a transcriptional activator as it does with the androgen receptor to stimulate androgen-receptor-dependent transcription. The posttranslational modifications made by LSD1 are completed through a FAD-dependent oxidative reaction. In addition to its ability to alter gene expression, LSD1 can also read histone marks on the Lys9-Ser10 locus and act as a docking module to stabilize corepressor complexes on chromatin. The demethylase is regulated by HDAC1/2, CoREST and BHC80. LSD1 is positively regulated and protected from proteasomal degradation by CoREST and is negatively regulated by BHC80.

LSD1 negatively regulates the tumor suppressor and transcriptional activator protein p53 by demethylating at K370 in vitro. The amine oxidase domain of LSD1 interacts with Snail1's SNAG domain to grant the transcriptional repressive function of Snail1 LSD1 has protein interactions with the HIV-1 protein, Tat. The finding that LSD1 activates the viral protein through demethylation suggests a therapeutic possibility through LSDT inhibition. Mutated variants of the KDM1A gene are associated with a developmental delay disorder known as Cleft palate, psychomotor retardation and distinctive facial features (CPRF).

LSD1 inhibition results in reduced herpesvirus lytic infection, subclinical shedding and reactivation due to the lack of LSD1 being able to form the DNA virus' crucial transcriptional coactivator complex. Contrary to the proviral transcriptional activator role that LSD1 has for DNA viruses, it exhibits antiviral effects against RNA viruses. A common host restriction factor for RNA viruses known as IFITM3 is demethylated and consequently activated by LSD1. It has been demonstrated that LSD1 restricts influenza A virus infection by demethylating IFITM3 at position K88. These findings suggest that LSD1 activity is advantageous to the host when fighting an RNA virus.

Target Relevance

LSD1 possesses antiviral activity against RNA viruses because of its association with IFITM3. IFITM3 belongs to the interferon-inducible transmembrane gene family that is one of many small interferon stimulated genes (ISGs) that inhibit early stages of viral life cycles. ISGs produce cellular factors that carry out the type I interferon (IFN) response that serves to defend cells from invasive viruses. Given the known antiviral effect that IFITM3 has against a range of RNA viruses, it is possible that IFITM3 could exhibit this same effect against the novel coronavirus SARS-CoV-2; as it is a positive-sense, single-stranded RNA virus. Without being bound by any particular theory, it is believed that LSD1 can mediate the antiviral properties of IFITM3 through demethylation at site K88, thereby contributing to the antiviral effect against the SARS-CoV-2. The negative effect on cell viability in response to LSD1 LOF demonstrates that LSD1 has antiviral properties. Compounds capable of targeting KDM1A that exhibit sufficient antiviral activity (log₂FC<2*Stdv of negative control DMSO) and sufficiently low cytotoxicity (normalized cell number>0.65) were identified in Example 12 and are listed in Table 3.

TABLE 3
Compounds Targeting KDM1A

| Drug Name | Synonyms | CAS | Drugbank ID | Formula |
|---|---|---|---|---|
| etonogestrel | 3-Ketodesogestrel; 3-Oxodesogestrel; | 54048-10-1, | DB00294 | C22H28O2 |
| | Etonogestrel; tonogestrel; | 54048-10-1 | | |
| | Etonogestrelum, EluRyng; | 131562-74-8 | | |
| | Etonogestrel/Ethinyl Estradiol; Implanon; | 135988-10-2 | | |
| | Nexplanon; NuvaRing; Nuvaring | 165050-21-5 | | |
| desogestrel, | Cerazette, Mircette, Org-37652 Org- | 54024-22-5 | 304 | C₂₂H₃₀O |
| desogestrel | 2969 | | | |

### Type H. Proviral Factors

The following are non-limiting examples of target host factors with proviral activities and favorable characteristics for further development.

### Target 4. Baculoviral IAP Repeat-Containing Protein 2

Identification

UniProtKb: Q13490 (BIRC2_HUMAN)

Gene: BIRC2

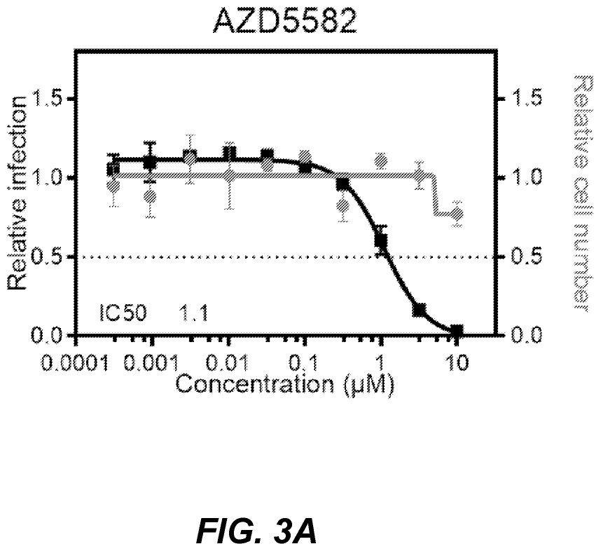

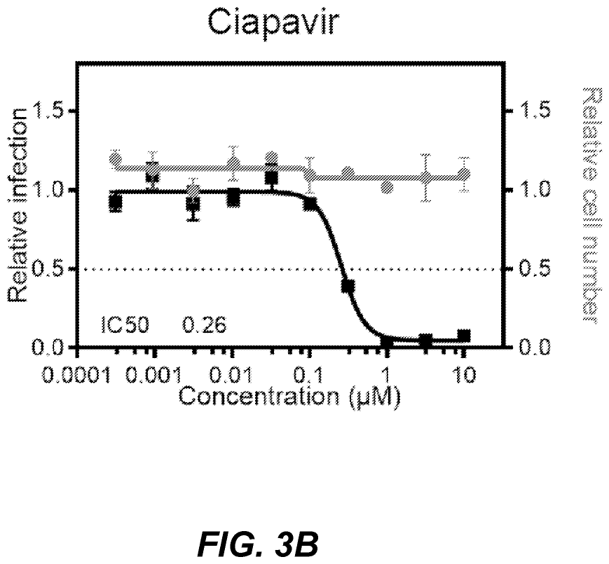

Evidence: primary siRNA screen and validation screen described herein, and Smac-mimetic drugs showed efficacy (FIGS. 3A-B).

Alternative Names/Synonyms

API1, HIAP2, Hiap-2, MIHB, RNF48, c-IAP1, cIAP1, baculoviral IAP repeat-containing protein 2, IAP homolog B, IAP-2, NFR2-TRAF signaling complex protein, RING finger protein 48, RING-type E3 ubiquitin transferase BIRC2, TNFR2-TRAF-signaling complex protein 2, apoptosis inhibitor 1, cellular inhibitor of apoptosis 1, inhibitor of apoptosis protein 2

Structure and Sequence

Baculoviral IAP repeat-containing protein 2, also known as cellular inhibitor of apoptosis 1 (C-IAP1), contains three baculoviral repeats (BIR) domains, a caspase recruitment domain, a C-terminal RING finger. C-IAP1 is distinct from other LAP proteins given that its third BIR domain is stabilized by a tetrahedrally-coordinated zinc. The BIR domain localizes the protein to the nucleus while the CARD domains aids in protein stabilization and of E3 ubiquitin-protein ligase activity inhibition by, “preventing dimerization of RING and binding and activation of E2 ubiquitin donor”. The three-dimensional structure can be viewed here, https://www.rcsb.org/structure/1QBH and https://www.rcsb.org/structure/3D9T. Its sequence, which is described in detail here: https://www.ebi.ac.uk/ena/data/view/U37547, produces two isoforms as a result of alternative splicing.

Known Locations and Abundance

This multi-functional protein is mainly expressed in adult skeletal muscle, thymus, testis, ovary and pancreas. Little to no CIAP-1 is found in the brain and peripheral blood leukocytes. (UniProt). CIAP-1 is localized almost exclusively to the nuclear compartment of cells where it has a role in cell cycle regulation.

Physiology and Disease

Encoded by the BIRC2 gene located on chromosome 11, C-IAP1 belongs to a family of proteins referred to as IAPs that function to inhibit apoptosis. These proteins do so by acting on caspases which are responsible for inducing apoptosis. More specifically, BIRC2 blocks the activation of caspase 3,7 and 9 to prevent programmed cell death. The BIR, RING and CARD domains function to inhibit apoptosis, possess E3 ubiquitin ligase activity and regulate proteasomal degradation and ubiquitination of its substrates, and mediate protein-protein interactions that cause oligomerization with other proteins, respectively. While there is more than one BIR domain in CIAP-1, the BIR3 is of special interest for inhibitor design. Cell death can be induced by inhibitors that predominantly bind to the BIR3 domain of C-IAP1 by inducing proteasome-mediated degradation. In addition to preventing apoptosis, BIRC2 serves a purpose in inflammatory and mitogen kinase signaling, immunity, cell proliferation cell invasion and metastasis.

Abnormal regulation of the BIRC2 gene could lead to cancer or neurodegenerative diseases. When CIAP-1 is overexpressed, the resultant genetic instability points to implications for cancer. Many kinds of cancer show upregulation of C-IAP1 levels. This family of proteins' role in cell death has led scientists to target them for anticancer therapy through inhibition with hopes to trigger apoptosis in cancerous cells. Other researchers believe that C-IAP1 functions as an oncogene by promoting the degradation of a crucial regulator in the Myc pathway.

Target Relevance

(1) The apoptotic inhibiting function of IAPs has been identified to occur in Hepatitis B Virus-infected models. These proteins, including C-IAP1, restrict TNF-mediated HBV clearance. Without being bound by any particular theory, these findings suggest that IAP inhibitors could be useful to allow for viral clearance and programmed cell death of infected cells.

(2) Beyond the scope of the apoptotic inhibition function of C-IAP1, the ubiquitination function of this protein serves an antiviral purpose. Type I interferon (IFNs) activation is vital for the antiviral innate immune response. In virus-infected models, it has been shown that the E3 ubiquitin ligases, C-IAP1 and C-IAP2, are responsible for the ubiquitination of TRAF3 and TRAF 6. This action is significant for type I interferon induction and proper cellular antiviral response. Without being bound by any particular theory, it is believed that C-IAP1 can also demonstrate this form of crucial ubiquitination on the same factors in response to SARS-CoV-2 infection, in efforts to induce IFN signaling and production to combat the virus. Compounds capable of targeting BIRC2 that exhibit sufficient antiviral activity (log₂FC<2*Stdv of negative control DMSO) and sufficiently low cytotoxicity (normalized cell number>0.65) were identified in Example 12 and are listed in Table 4.

TABLE 4
Compounds Targeting BIRC2

| | | | Drugbank | |
|---|---|---|---|---|
| Drug Name | Synonyms | CAS | ID | Formula |
| xaliproden, | Xaliproden | 135354-02-8 | DB06393 | C24H22F3N |
| xaliproden | | 90494-79-4 | | |
| hydrochloride | | | | |
| licofelone, [6-(4- | Licofelone | 156897-06-2 | DB04725 | C23H22ClNO2 |
| chlorophenyl) -2,2- | | | | |
| dimethyl-7- | | | | |
| pheny1-2,3 - dihydro- | | | | |
| 1h- pyrrolizin-5-yl] | | | | |
| acetic acid, | | | | |
| licofelone, ML- 3000 | | | | |
| clidinium, clidinium | Ro-2-3773, Quarzan | 7020-55-5, | | C₂₂H₂₆NO₃+ |
| bromide | | 3485-62-9 | | |
| | | 7020-55-5 | | |
| | | 7166-86-1 | | |
| | | 8015-20-1 | | |
| | | 76404-05-2 | | |
| | | 53950-10-0 | | |
| | | 70761-71-6 | | |
| | | 71861-83-1 | | |
| | | 83903-46-2 | | |
| | | 85344-78-1 | | |
| | | 88498-51-5 | | |
| | | 98253-50-0 | | |
| ivacaftor, ivacaftor, | Ivacaftor; Ivacaftorum; N- | 873054-44-5 | DB08820 | C24H28N2O3 |
| ivacaftor (vx- 770) | (2,4-di-tert-butyl-5- | | | |
| | hydroxyphenyl)-4-oxo- 1,4- | | | |
| | dihydroquinoline-3- | | | |
| | carboxamide | | | |
| ampicillin, ampicillin | ABPC; | 69-53-4, 69-52- | DB00415 | C16H19N3O4S |
| (sodium), ampicillin | Aminobenzylpenicillin; | 3 | | |
| sodium, sodium | Ampicilina; Ampicillin; | 37203-95-5 | | |
| (2s,5r,6r)-6- | Ampicillin (anhydrous); | 51004-51-4 | | |
| {[(2r)-2-amino- 2- | Ampicillin acid; Ampicillin | 52232-32-3 | | |
| phenylethanoyl] | anhydrous; Ampicillin, | 24840-26-4 | | |
| amino}-3,3- | anhydrous; Ampicilline; | 34205-43-1 | | |
| dimethyl-7-oxo- 4- | Ampicillinum; Anhydrous | 7177-48-2 | | |
| thia-1- azabicyclo | ampicillin; AP; D-( )-6-(- | 69-53-4 | | |
| [3.2.0]heptane- 2- | aminophenylacetamido) | 8067-85-4 | | |
| carboxylate, | penicillanic acid; D-( )- | 23277-71-6 | | |
| ampicillin sodium, | ampicillin | 33604-21-6 | | |
| ampicillin | | 37234-64-3 | | |
| trihydrate | | 47355-94-2 | | |
| | | 50584-05-9 | | |
| | | 800-79-3 | | |
| | | 8056-87-9 | | |
| | | 96707-69-6 | | |
| | | 98520-55-9 | | |
| | | 80042-41-7 | | |
| | | 33276-75-4 | | |
| | | 94935-63-4 | | |
| perindopril, | (2S,3aS,7aS)-1-[(2S)-2- | 82834-16-0 | DB00790 | C19H32N2O5 |
| perindopril erbumine, | {[(2S)-1-ethoxy-1- | | | |
| perindopril erbumine | oxopentan-2-yl]amino} | | | |
| (aceon), perindopril | propanoyl]octahydro-1H- | | | |
| (erbumine), | indole-2-carboxylic acid; | | | |
| perindopril erbumine | Perindopril; Perindoprilum | | | |
| perindopril, | Rupatadina; Rupatadine | 158876-82-5, | DB11614 | C26H26ClN3 |
| rupatadine | | 158876-82-5 | | |
| (fumarate), | | 156611-76-6 | | |
| rupatadine fumarate | | 182349-12-8 | | |
| halofantrine, | SK&F-102866 WR- | 69756-53-2, | 1218 | C₂₆H₃₀Cl₂F₃NO |
| halofantrine hcl, | 171669 Halfan | 36167-63-2 | | |
| halofantrine | HALOFANTRINE | 106927-11-1 | | |
| hydrochloride | HYDROCHLORIDE | 69756-53-2 | | |
| halofantrine | Halofantrina; Halofantrine; | 69756-53-2 | DB01218 | C26H30Cl2F3N O |
| | Halofantrinum, Halfan, | | | |
| | SK&F-102866 WR-171669 | | | |
| | Halfan HALOFANTRINE | | | |
| | HYDROCHLORIDE | | | |
| bedaquiline | 1-(6-Bromo-2-methoxy- | 843663-66-1 | DB08903 | C32H31BrN2O 2 |
| | quinolin-3-y1)-4- | | | |
| | dimethylamino-2- | | | |
| | naphthalen-1-yl-1-phenyl- | | | |
| | butan-2-ol; Bedaquilina; | | | |
| | Bedaquiline; Bdaquiline; | | | |
| | Bedaquilinum | | | |
| loratadine, loratidine, | Loratadina; Loratadine; | 79794-75-5, | DB00455 | C22H23ClN2O2 |
| loratadine, loratadine | Loratadinum | 79794-75-5 | | |
| | | 132316-36-0 | | |
| | | 156098-07-6 | | |
| osanetant | SR-142806 SR-142801 | 160492-56-8 | DB04872 | C35H41Cl2N3O 2 |
| talmapimod, scio 469 | Talmapimod | 309913-83-5 | DB05412 | C27H30ClFN4 O3 |
| hydrochloride, scio- | | | | |
| 469 | | | | |
| lonafarnib | Lonafarnib; Lonafarnibum, | 193275-84-2 | DB06448 | C27H31Br2ClN |
| | CH-59228 lonafarnib, | | | 4O2 |
| | Sarasar SCH- 58006 MK- | | | |
| | 6336 | | | |

### Target 5. Diacylglycerol Lipase Alpha (Human)

Identification

UniProtKb: Q9Y4D2 (DGLA_HUMAN)

Gene: C11ORF11, DAGLA

Evidence: primary siRNA screen, validation screen, and individual siRNA screen described herein

Alternative Names/Synonyms

C11orf11, DAGL(ALPHA), DAGLALPHA, NSDDR, snl-specific diacyiglycerol lipase alpha, DGL-alpha, neural stem cell-derived dendrite regulator

Structure and Sequence

DAGL alpha is composed of 1042 amino acids and is distinguishable from its gene duplicate DAGL beta by its large carboxy-terminal tail. Calcium acts as a cofactor for the enzyme and has optimal activity at a pH of 7.0 (UniProt). DAGL alpha is a serine hydrolase with a 4TM domain and a catalytic domain with an active sight that harbors hydrophobic character. Refer to Reisenberg, 2012 for a detailed explanation of the DAGL structure.

Known Locations and Abundance

The enzyme is highly expressed in the brain and pancreas. Expression differences in the brain/nervous system during development and adulthood are separated by axonal tracts versus synaptic fields, respectively.

Physiology and Disease

Diacylglycerol lipase alpha is encoded by one of the main endocannabinoid system genes, DAGLA. This enzyme's primary function is to synthesize 2-arachidonoylglycerol (2-AG) which is an important endogenous signaling ligand for the cannabinoid receptors 1 and 2. This endocannabinoid signaling system is required for proper axonal growth during brain development as well as production and migration of new neurons and retrograde synaptic signaling in adults. Knockout experiments in mice have proven that both DAGL alpha and DAGL beta produce the majority of 2-AG in the brain, spinal cord, liver and other tissues (Reisenberg et al., 2012). The KO experiment also showed that DAGL alpha is particularly important in areas with high synapse concentration. Additionally, DAGLs serve a role in the downstream production of inflammatory prostaglandins in the brain.

Abnormalities in the DAGLA gene can lead to a number of neurological, physiological and psychological complications. For instance, heterozygous rare variants in DAGLA were significantly associated with seizures and neurodevelopmental disorders such as autism and irregular brain morphology. The inflammatory pathway that DAGLA is involved in is also suggested to be a part of the pathogenesis of Parkinson's disease. Stress-induced responses and genetic dysfunction of the DAGLA gene is presumably involved in the development of alcoholism. Experimentally designed DAGLA knockdown models concluded that enzyme is likely a factor in tumoral progression and has potential to be a therapeutic target for oral squamous cell carcinomas. Knockout experiments of DAGLA deficient mice resulted in complete loss of synaptic plasticity and compromised adult neurogenesis in the hippocampus and subventricular zone. It is also believed that a genetic defect on chromosome 11q12 is responsible for spinocerebellar ataxia type 20 due to a duplicated segment that includes DAGLA. DAGLA has been shown to be upregulated in Ebola virus-infected cells in comparison to controls

Target Relevance

Without being bound by any particular theory, DAGLA's role in the inflammatory pathway is believed to be related to an upregulation in response to SARS-CoV-2 infection, in efforts to notify and support the organism's innate immunological reaction. However, excessive inflammatory prostaglandin production could end up having negative consequences on the host. Compounds capable of targeting DAGLA that exhibit sufficient antiviral activity (log₂FC<2*Stdv of negative control DMSO) and sufficiently low cytotoxicity (normalized cell number>0.65) were identified in Example 12 and are listed in Table 5.

TABLE 5
Compounds Targeting DAGLA

| Drug Name | Synonyms | CAS | Drugbank ID | Formula |
|---|---|---|---|---|
| mk-0752 | c-7617 cis-4-[(4-Chlorophenyl) | 471905-41-6 | DB12852 | C21H21ClF2O4 S |
| | sulfonyl]-4-(2,5-difluorophenyl) | | | |
| | cyclohexanepropanoic acid L- | | | |
| | 000891675 MK 0752 mk-0752 | | | |
| | MK0752 | | | |
| daprodustat | GSK-1278863 (topical) daprodustat | 960539-70-2 | DB11682 | C19H27N3O6 |
| | (suspension) 1278863A (oral) GSK | | | |
| | 1278863 (solution) JVR38ZM64B | | | |
| | 278863 GSK-1278863A (oral) | | | |
| | 1278863 (oral) | | | |
| cintirorgon | LYC-55716 | 2055536-64- | | C₂₇H₂₃F₆NO₆S |
| | | 4 | | |
| halofantrine | Halofantrina; Halofantrine; | 69756-53-2 | DB01218 | C26H30Cl2F3N O |
| | Halofantrinum, Halfan, SK&F- | | | |
| | 102866 WR-171669 Halfan | | | |
| | HALOFANTRINE | | | |
| | HYDROCHLORIDE | | | |
| halofantrine, | SK&F-102866 WR- | 69756-53-2, | 1218 | C₂₆H₃₀Cl₂F₃NO |
| halofantrine hcl, | 171669 Halfan HALOFANTRINE | 36167-63-2 | | |
| halofantrine | HYDROCHLORIDE | 106927-11-1 | | |
| hydrochloride | | 69756-53-2 | | |
| dinoprost | Dinoprost trometamol; PGF2-alpha | 36300-07-9 | DB01160, | C24H45NO8, |
| tromethamine, | THAM; PGF2A THAM; PGF2alpha | 38473-38-0 | DB12789 | C20H34O5 |
| dinoprost | THAM; Prostaglandin F2-alpha | 38562-01-5 | | |
| | THAM; Prostaglandin F2a | 51553-13-0 | | |
| | tromethamine; Prostaglandin F2alpha | 101913-98-8 | | |
| | tham; Prostin F2 Alpha, Dinoprost; | 4510-16-1 | | |
| | dinoprosta, Dinolytic; Enzaprost F; | 13535-33-6 | | |
| | Prosmon; Prostin F2 Alpha | 551-11-1 | | |
| | | 99437-94-2 | | |
| | | 23518-25-4 | | |
| | | 27415-26-5 | | |
| | | 42586-88-9 | | |
| | | 51830-21-8 | | |
| | | 58717-39-8 | | |
| pci-27483 | | 871266-63-6 | DB13000 | C26H24N6O9S |

### Target 6 Calpain-1 Catalytic Subunit

Identification

UniProtKb: P07384 (CAN1_HUMAN)

Gene: CAPN1

Evidence: primary siRNA screen and validation screen described herein

Alternative Names/Synonyms

CANP, CANP1, CANPL1, SPG76, muCANP, muCL, calpain-1 catalytic subunit, CANP 1, calcium-activated neutral proteinase 1, calpain 1, (mu/I) large subunit, calpain mu-type, calpain, large polypeptide Li, calpain-1 large subunit, cell proliferation-inducing gene 30 protein, cell proliferation-inducing protein 30, micromolar-calpain

Structure and Sequence

The CAPN1 gene encodes the Calpain-1 80-kDa catalytic subunit which, in combination with a 30-kDa regulatory subunit, makes up a heterodimeric intracellular protease. The cysteine protease is calcium-dependent and functional at a neutral pH. Upon Ca2+ binding, a conformational change occurs and results in greater accessibility of the active site in the catalytic domain. The catalytic domain of human Calpain-1 can be viewed here, https://www.rcsb.org/structure/2ARY and the crystal structure here, http://www.sciencedirect.com/science/article/pii/S0022283606015774. Refer to https://pubmed.ncbi.nlm.nih.gov/9271093/ for the calcium-binding properties and sites of human calpain.

Known Locations and Abundance

CAPN1 is located on chromosome 11q13 in humans and is expressed ubiquitously. Calpain-1 is localized in synaptic compartments and translocate from the cytoplasm to the plasma membrane once activated by sufficient calcium.

Physiology and Disease

Belonging to the Calpain family, Calpain-1 is a calcium-dependent protease possessing enzymatic activity that is necessary for the induction of long-term potentiation and is neuroprotective. The calpain signaling cascades are associated with synaptic plasticity, cytoskeletal regulation, AMPA receptor trafficking, inflammatory processes, actin polymerization and regulation of nearby protein synthesis. Once activated by micromolar concentrations of calcium, the enzyme cleaves its substrates such as cytoskeletal and submembranous proteins or FLG2, through proteolytic processing. Calpastatin is able to regulate calpain activity, as it is a specific endogenous inhibitor.

Whole-exome sequencing of individuals diagnosed with hereditary spastic paraplegia (HSP) revealed that mutations in CAPN1 are widely responsible for disease. Calpain-1 deficient models demonstrated the characteristics of HSP such as: neuronal and axonal dysfunction and degeneration, locomotor defects and axonal abnormalities, abnormal branchiomotor neuron migration and disorganized acetylated-tubulin axonal networks in the brain.

Calpains are known to be involved in virus-induced apoptotic myocardial injury, HIV and echovirus 1 replication, and the regulation of herpes simplex virus intracellular migration. Calpain also serves an interesting role in the pathogenesis of the Influenza A virus (IAV). Following infection by IAV, Ca2+ mobilization occurs which can activate the calcium-dependent calpain proteases. The active calpain then transduces downstream signaling that results in the expression of chemokines, cytokines and recruitment of immune cells to lung tissue. IAV replication within bronchial epithelial cells has been found to be promoted by calpains as they support the beginnings of the viral life cycle. Calpain inhibition in IAV-infected models resulted in decreased viral replication and host mortality thereby confirming the importance of calpain activity for viral pathogenesis. In instances where viral infection induces pneumonia or any respiratory distress, calpain inhibition could be advantageous.

Target Relevance

Simeprevir, a calpain IV and cathepsin F inhibitor, is being sought after for repurposing against SARS-Cov-2. Without being bound by any particular theory, it is believed that a small molecule inhibitor of calpain-1 disclosed herein can have the same results in the novel coronavirus SARS-CoV-2, given the existing data that calpain inhibition restricts viral replication and propagation. Compounds capable of targeting CAPN1 that exhibit sufficient antiviral activity (log₂FC<2*Stdv of negative control DMSO) and sufficiently low cytotoxicity (normalized cell number>065) were identified in Example 12 and are listed in Table 6.

TABLE 6
Compounds Targeting CAPN1

| Drug Name | Synonyms | CAS | Drugbank ID | Formula |
|---|---|---|---|---|
| mecillinam, (2s, | Ro-10-9070 Selexidine amdinocillin | 32887-01-7, | 1163 | C₁₅H₂₃N₃O₃S |
| 5r,6r)-6-{[(e)-1- | Coactin Ro- 109070 | 32887-01-7 | | |
| azepanylmethyl- | | 32887-08-4 | | |
| idene]amino}- | | 51556-89-9 | | |
| 3,3-dimethyl-7- | | 70178-27-7 | | |
| oxo-4-thia-1- | | 73937-07-2 | | |
| azabicyclo | | 79580-20-4 | | |
| [3.2.0]heptane- | | | | |
| 2-carboxylic | | | | |
| acid, mecillinam | | | | |
| psi-7976, psi- | Sofosbuvir, Sovaldi (tablet) sofosbuvir | 1190307-88-0 | 8934 | C₂₂H₂₉FN₃O₉P |
| 7977 | (tablet) SoviHep GS-7977 PSI-7977, | | | |
| | Elbanovir GI 7977 GI-7977 GS-7977 | | | |
| | isopropyl | | | |
| | (2S)-2-{[(S)-{[(2R,3R,4R,5R)-5-(2,4- | | | |
| | dioxo-3,4- | | | |
| | dihydropyrimidin-1(2H)-yl)-4-fluoro-3- | | | |
| | hydroxy-4- methyltetrahydrofuran-2- | | | |
| | yl]methoxy}(phenoxy) | | | |
| | phosphoryl]amino} propanoate | | | |
| | PSI 7977 PSI-7977 Resof | | | |
| | S)-Isopropyl 2-((S)-(((2R,3R,4R,5R)-5- | | | |
| | (2,4-dioxo-3,4- | | | |
| | dihydropyrimidin-1(2H)-yl)-4-fluoro-3- | | | |
| | hydroxy-4- methyltetrahydrofuran-2- | | | |
| | yl)methoxy)- | | | |
| | (phenoxy)phosphorylamino)propanoate | | | |
| | Soforal Sofosbuvir Sovaldi Sovaldi ® | | | |

### Target 7. Lysine-Specific Demethylase 5B

Identification

UniProtKb: Q9UGL1 (KDM5B_HUMAN)

Gene: KDM5B

Evidence: primary siRNA screen, validation screen, and individual siRNA screen described herein

Alternative Names/Synonyms

CT31, JARIDIB, MRT65, PLU-1, PLU1, PPP1R98, PUT1, RBBP2H1A, RBP2-H1, lysine-specific demethylase 5B, cancer/testis antigen 31, histone demethylase JARID1B, jumonji, AT rich interactive domain 1B, jumonji/ARID domain-containing protein 1B, lysine (K)-specific demethylase 5B, protein phosphatase 1, regulatory subunit 98, putative DNA/chromatin binding motif, retinoblastoma-binding protein 2 homolog 1, retinoblastoma-binding protein 2, homolog 1A

Structure and Sequence

Lysin-specific demethylase 5B, also known as PLU-1, is a 1544 amino acid multidomain protein that is encoded by the KDM5B gene located on chromosome 1. Alternative splicing of the sequence produces 2 isoforms. This multidomain protein binds 1 Fe²⁺ per subunit and is dependent on iron and 2-oxoglutarate. As a member of the KDM5 family, this enzyme has an N-terminal Jumonki (JmjN) domain, a DNA-binding ARID domain (AT-rich interactive domain), a catalytic JmjC domain, a C5HC2 zinc finger motif, a PLU1 motif and two to three methyl-lysine or methyl-arginine PHD domains. The three-dimensional structure of this demethylase can be found here, http://www.rcsb.org/structure/5A3Nn, the catalytic domain http://www.rcsb.org/structure/5A1F and the structural analysis described in (Johansson et al., 2016).

Known Locations and Abundance

PLU-1 is ubiquitously expressed but is most abundant in the testis. Abnormal expression is observed in various types of cancer. The protein is localized exclusively in the nucleus (UniProt).

Physiology and Disease

As a member of the KDM5 subfamily, lysine-specific demethylase 5B (PLU-1 or JARIDIB) is considered a transcriptional corepressor that participates in epigenetic regulation. The main function of this oxygenase is the catalyzed demethylation of all possible methylation sites from lysine 4 of histone H3, which are located at the transcriptional start site of activated genes. As a result of this hydroxylation reaction, PLU-1 regulates cell proliferation and stem cell self-renewal and differentiation, in terms of development. KDM5B is an important factor in proper neural differentiation and control of cellular senescence. It is speculated that this protein participates in genome stability and DNA repair (NCBI gene). Its transcriptional repressor properties have been identified to interact with BF-1 and PAX9 suggesting a role in groucho-mediated transcriptional repression.

Multiple studies have found that PLU-1 influences cancer cell proliferation, reduces the expression of tumor suppressor genes, promotes the development of drug tolerance sustains tumor-initiating cells. More specifically, this gene is upregulated in human breast cancers and the testis. PLU-1 influences the proliferative properties of breast cancer cells by repressing BRCA1, CAV1, 14-3-3-sigma and HOXA5. Lastly, a mutation in the KDM5B gene is associated with mental retardation, autosomal recessive 65 causing developmental delay, facial dysmorphism and camptodactyly. Research efforts are seeking to develop compounds that can inhibit the negative effects of PLU-1 for anticancer therapeutics.

Target Relevance

KDM5B has a role in cellular senescence by acting through: direct regulation of pRb, the p16/pRb pathway, p53/p21 pathway, regulation of CDKN2A locus. This H3K4 demethylase modulates gene expression that is oftentimes involved in senescence-associated cell cycle arrest. Cellular senescence is a stress response cellular mechanism that occurs to prevent proliferation of damaged or infected cells, as an immune defense by recruiting immune cells and as an antiviral response. Cellular senescence acts as an antiviral defense mechanism with the end objective of clearing out infected cells. This effect has also been observed in cancerous cell-lines where only depletion of KDM5B or inhibition of the gene product granted tumor eradication. The success and host benefits of senescence-mediated antiviral activity has been validated in vivo. The harmful effects that KDM5B has in cancerous and viral-infected models, can be additive in combination as seen with Hepatocellular carcinoma cells that are derived from tissue samples infected with hepatitis B virus (HBV) or hepatitis C virus (HCV). Without being bound by any particular theory, in viral-infected models such as SARS-CoV-2, small molecule inhibition of the demethylase, KDM5B (also known as JARID1B), would allow proper recruitment and action of immune cells to eradicate the virus and prevent further viral replication and propagation. Compounds capable of targeting KDM5B that exhibit sufficient antiviral activity (log₂FC<2*Stdv of negative control DMSO) and sufficiently low cytotoxicity (normalized cell number>0.65) were identified in Example 12 and are listed in Table 7.

TABLE 7
Compounds Targeting KDM5B

| Drug Name | Synonyms | CAS | Drugbank ID | Formula |
|---|---|---|---|---|
| pik-294 | | 900185-02-6 | | C₂₈H₂₃N₇O₂ |
| dadle, bw-180c | [D-Ala2, D-Leu5]-Enkephalin; H- | | DB08856 | C29H39N5O7 |
| | Tyr-D-Ala-Gly-Phe-D-Leu; L- | | | |
| | Tyrosyl-D-alanylglycyl-L- | | | |
| | phenylalanyl-D-leucine | | | |
| oxytetracycline, | 5-Hydroxytetracycline; | 79-57-2 | 595 | C₂₂H₂₄N₂O₉ |
| oxytetracycline | Embryostat; | | | |
| (terramycin), | Hydroxytetracyclinum; Mepatar; | | | |
| oxy tetracycline | Ossitetraciclina; Oxitetraciclina; | | | |
| dihydrate | Oxyterracin; Oxyterracine; | | | |
| | Oxytetracyclin; Oxytetracycline; | | | |
| | Oxyttracycline; Oxytetracycline | | | |
| | (anhydrous); Oxytetracycline | | | |
| | amphoteric; Oxytetracyclinum, | | | |
| | Terra-Cortril; Terramycin; | | | |
| | Urobiotic, Adamycin; | | | |
| | Anglocycline; Corsamycin; | | | |
| | Oxacin; Oxypan; Oxyterracyna; | | | |
| | Terrados; Tetran; Tetrasona | | | |
| cefonicid, | (6R,7R)-7-{[(2R)-2-hydroxy-2- | 61270-58-4, | DB01328 | C18H18N6O8S 3 |
| cefonicid sodium, | phenylacetyl]amino}-8-oxo-3- | 61270-78-8 | | |
| cefonicid (sodium) | ({[1-(sulfomethyl)-1H-tetrazol-5- | 61270-58-4 | | |
| | yl]sulfanyl}methyl)-5-thia-1- | 71420-79-6 | | |
| | azabicyclo[4.2.0]oct-2-ene-2- | | | |
| | carboxylic acid; Cefonicid; | | | |
| | Cefonicido; Cefonicidum | | | |

### Target 8. Mitogen-Activated Protein Kinase 11

Identification

UniProtKb: Q15759 (MK11_HUMAN)

Gene: MAPK11

Evidence: primary siRNA screen, validation screen, and individual siRNA screen described herein

Alternative Names/Synonyms

P38B3, P38BETA2, PRKM11, SAPK2, SAPK2B3, p138-2, p38Beta, mitogen-activated protein kinase 11, MAP kinase 11, MAP kinase p38 beta, mitogen-activated protein kinase p38 beta, mitogen-activated protein kinase p38-2, stress-activated protein kinase-2, stress-activated protein kinase-2b

Structure and Sequence

The Mitogen-activated protein kinase 11 is encoded by the MAPK11 gene located on chromosome 22. Two predominant isoforms are a result of alternative splicing. MAPK11, also referred to as p38β, is a serine-threonine kinase that serves a vital role in the signal transduction pathway that responds to extracellular stimuli such as proinflammatory cytokines or stress responses. The sequence of MAPK can be found here, https://www.ncbi.nlm.nih.gov/protein/Q15759_and the detailed protein structure here https://www.rcsb.org/pdb/protein/Q15759_and https://www.rcsb.org/structure/3GP0.

Known Locations and Abundance

P38beta is expressed highest in the brain and heart but is also expressed in placenta, lung, liver, skeletal muscle, kidney and pancreas (UniProt). The kinase is found to exist in the nucleus and cytoplasm.

Physiology and Disease

P38beta MAPK11 is activated by a number of factors like cytokines, physical environmental stress stimuli, and LPS that in turn phosphorylate the threonine and tyrosine residues. Once activated, the kinase is then able to activate a large quantity of transcription factors; for example, ADAM17, FGFR1, ATF1, TNF-alpha and activating transcription factor-2 (ATF-2). TNF-alpha acts to produce inflammatory cytokines, upregulate adhesion molecules, proliferation, differentiation and cell death whereas ATF-2 is involved in the cell cycle, differentiation, transformation and immunological responses. Inflammatory gene expression, of IL6, IL8 and IL12B for instance, is modulated by the complex MAPK signaling cascades. In addition to the abilities MAPK11 possesses in the nucleus, it also is able to influence protein turnover in the cytoplasm and modulate ectodomain shedding of transmembrane proteins (UniProt).

A study in monocytic cells revealed that histone deacetylase HDAC3 interacts with MAPK11 by repressing MAPK11/ATF-2 signaling. By inhibiting MAPK11, the transcriptional activity of ATF-2 is not able to regulate TNF expression. Therefore, HDAC3 modulates TNF expression and controls the gene expression of proinflammatory cytokines. Inflammatory stimuli can hyperactivate the immune system if an excessive amount of TNF is produced. By decreasing the phosphorylation state of MAPK11 that is necessary to transform the kinase into its activated form, HDAC3 is able to consequently repress the downstream TNF gene expression. These findings further confirm MAPK11's attributes to inflammatory and immune responses. An example therapy being researched that involves the MAPK signaling pathway in response to environmental stimuli is inhibition of p38b to treat severe asthma. Clinical trial NCT00676572 is inhibiting this signal cascade responsible for the inflammatory process under this condition.

P380 has an influential role in osteolytic bone destruction in patients diagnosed with bone metastatic breast cancer. Knockdown experiments of p38β demonstrated reduced osteoclast differentiation and bone destruction in breast cancer cells and SCID mouse models, respectively. This kinase increases the expression and secretion of monocyte chemotactic protein-1 which in turn activates osteoclast differentiation and activity. These findings, described in He et al., 2014, have resulted in seeking p38β inhibition as a potential therapeutic in treating bone-metastatic breast cancer.

The serine-threonine kinase is also tied to myelodysplastic syndromes (MDS) and acute myeloid leukemia (AML). These conditions induce a pathogenic inflammatory response in bone marrow. MAPK p38's overexpression in these conditions has led to subsequent research towards inhibiting the proinflammatory kinase's effects. Pexmetinib, an inhibitor of both Tie2 and p38 MAPK, blocked leukemic proliferation, prevented activation of downstream effector kinases and evaded the effects of TNF-alpha on healthy hematopoietic stem cells. The successful inhibition of malignant cells and reversed cytokine-induced suppression indicated that dual inhibition of Tie2 and p38 MAPK could be beneficial for the treatment of MDS and AML.

It has been reported that MAPK11 has interactions with the following HIV-1 proteins: envelope surface glycoprotein gp120, Nef, Tat and Vpr (NCBI Gene). MAPK11 was also found to participate in a signal transduction pathway that involves the promyelocytic leukemia protein and HDAC3 (Prospecbio).

Target Relevance

MAPK11 has an important role in the inflammatory response pathway. Its activation functions to in turn activate TNFa and ATF-2, among other transcription factors, which both serve multiple purposes involved in the immune system's response to infection. Without being bound by any particular theory, it is believed that MAPK11 can also exhibit this same key role in the immune system's fight against SARS-CoV-2 infection, however it can cause hyperactivation of the host immune system and end up causing more harm than benefit. Without being bound by any particular theory, it is believed that P38 kinase activities can also be employed by SARS-CoV-2 for viral reproduction and propagation as it has been found to with the human cytomegalovirus. Compounds capable of targeting MAPK11 that exhibit sufficient antiviral activity (log₂FC<2*Stdv of negative control DMSO) and sufficiently low cytotoxicity (normalized cell number>0.65) were identified in Example 12 and are listed in Table 8.

TABLE 8
Targeting MAPK11

| Drug Name | Synonyms | CAS | Drugbank ID | Formula |
|---|---|---|---|---|
| adx-47273 | | 851881-60-2 | | C₂₀H₁₇F₂N₃O₂ |
| tiaprofenic acid, | 2-(5-Benzoyl-thiophen-2-yl)-propionic | 33005-95-7, | 1600 | C₁₄H₁₂O₃S |
| 2-(5-benzoyl-2- | acid 2-(5-Benzyl-2-thienyl)propionsaeure | 33005-95-7 | | |
| thienyl) | 5- Benzoyl-alpha-methyl-2- | 59173-01-2 | | |
| propanoic acid, | thiopheneacetic acid 5-Benzoyl-alpha- | 62715-04-2 | | |
| tiaprofenic acid | methylthiophene-2- acetic acid | 103667-49-8 | | |
| | Acidetiaprofenique Acido tiaprofenico | 103667-50-1 | | |
| | Acidum tiaprofenicum Albert Albert | | | |
| | (capsule) Albert (tablet) alpha- Methyl- | | | |
| | 5-benzoyl-2-thienylacetic acid Artiflam | | | |
| | Artiflam (capsule) Artiflam (tablet) FC- | | | |
| | 3001 Flamirex RU-15060 RU-15060 | | | |
| | (capsule) RU-15060 (tablet) Suralgan | | | |
| | Surgam Surgam (capsule) Surgam | | | |
| | (tablet) Surgam SA Surgam SA (capsule) | | | |
| | Surgam SA (tablet) Surgamic Surgamic | | | |
| | (capsule) Surgamic (tablet) Surgamyl | | | |
| | Surgamyl (capsule) Surgamyl (tablet) | | | |
| | Tiafen Tiafen (capsule) Tiafen (tablet) | | | |
| | Tiaprofenic acid tiaprofenic acid | | | |
| | (capsule) tiaprofenic acid (tablet) | | | |
| | Tiaprofensaeure Tiaprofensaure | | | |

### Methods and Compositions

Disclosed herein include methods for preventing, delaying the onset of, or treating an infection or a disease caused by a RNA virus. In some embodiments, the method comprises: administering to a subject in need thereof a composition comprising a compound or a pharmaceutically acceptable salt, ester, solvate, stereoisomer, tautomer, or prodrug thereof, thereby preventing, delaying the onset of, or treating the infection or the disease, wherein the compound is selected from the compounds listed in Tables 2-8.

Disclosed herein include methods for preventing, delaying the onset of, or treating an inflammatory effect of an infection or a disease caused by a RNA virus. In some embodiments, the method comprises: administering to a subject in need thereof a composition comprising a compound or a pharmaceutically acceptable salt, ester, solvate, stereoisomer, tautomer, or prodrug thereof, thereby preventing, delaying the onset of, or treating the inflammatory effect, wherein the compound is selected from the compounds listed in Tables 2-8. The inflammatory effect can comprise respiratory failure, a sequela of respiratory failure, acute lung injury, or acute respiratory distress syndrome. The sequela of respiratory failure can comprise multi-organ failure. The composition can comprise a therapeutically or prophylactically effective amount of the compound.

The composition can be a pharmaceutical composition comprising the compound and one or more pharmaceutically acceptable excipients. The method can comprise: administering to the subject one or more additional antiviral agents. In some embodiments, at least one of the one or more additional antiviral agents can be co-administered to the subject with the composition. In some embodiments, at least one of the one or more additional antiviral agents can be administered to the subject before the administration of the composition, after the administration of the composition, or both. The composition can comprise one or more additional therapeutic agents. The one or more additional therapeutic agents can comprise one or more antiviral agents.

The composition can be administered to the subject by intravenous administration, nasal administration, pulmonary administration, oral administration, parenteral administration, or nebulization. The composition can be aspirated into at least one lung of the subject. The composition can be in the form of powder, pill, tablet, microtablet, pellet, micropellet, capsule, capsule containing microtablets, liquid, aerosols, or nanoparticles. The composition can be in a formulation for administration to the lungs. The composition can be administered to the subject once, twice, or three times a day. The composition can be administered to the subject once every day, every two days, or every three days. The composition can be administered to the subject over the course of at least two weeks, at least three weeks, at least four weeks, or at least five weeks.

The method can comprise: measuring the viral titer of the RNA virus in the subject before administering the composition to the subject, after administering the composition to the subject, or both. The viral titer can be lung bulk virus titer. In some embodiments, administrating the composition results in reduction of the viral titer of the RNA virus in the subject as compared to that in the subject before administration of the composition. The method can comprise: determining global virus distribution in the lungs of the subject. The method can comprise: measuring a neutrophil density within the lungs of the subject. In some embodiments, administering the composition results in reduction of the neutrophil density within the lungs of the subject as compared to that in the subject before administration of the composition. The method can comprise: measuring a total necrotized cell count within the lungs of the subject. In some embodiments, administering the composition results in reduction of the total necrotized cell count in the subject as compared to that in the subject before administration of the composition. The method can comprise: measuring a total protein level within the lungs of the subject. In some embodiments, administering the composition results in reduction of the total protein level within the lungs of the subject as compared to that in the subject before administration of the composition.

The subject in need thereof can be a subject that is suffering from the infection or the disease, or a subject that is at a risk for the infection or the disease. The infection or the disease can be in the respiratory tract of the subject. In some embodiments, the subject has been exposed to the RNA virus, is suspected to have been exposed to the RNA virus, or is at a risk of being exposed to the RNA virus. The subject can be a mammal (e.g., a human). The RNA virus can be a double-stranded RNA virus. The RNA virus can be a positive-sense single-stranded ssRNA virus. The positive-sense single-stranded ssRNA virus can be a coronavirus. The coronavirus can be an alpha coronavirus, a beta coronavirus, a gamma coronavirus, or a delta coronavirus. The coronavirus can be Middle East respiratory coronavirus (MERS-CoV), severe acute respiratory syndrome coronavirus (SARS-CoV), or SARS-CoV-2. The disease can be common cold, influenza, SARS, coronaviruses, COVID-19, hepatitis C, hepatitis E, West Nile fever, Ebola virus disease, rabies, polio, or measles.

The compound from Tables 2-8 and analogues thereof, a pharmaceutically acceptable salt of the compound from Tables 2-8 and analogues thereof, and/or a prodrug of the compound from Tables 2-8 and analogues thereof can be used in the methods and compositions disclosed herein for preventing, delaying the onset of, or treating an infection or a disease caused by a RNA virus (e.g., SARS-CoV-2). In some embodiments, one or more compounds disclosed in Tables 2-8 (or derivatives thereof) modulate host factors that govern SARS-CoV-2 replication, and thereby prevent and/or treat SARS-CoV-2 infection.

The compound from Tables 2-8 and analogues thereof are known and well described in the art. The structures and properties of the compound from Tables 2-8 and analogues thereof, and exemplary suitable chemical synthesis processes for these compounds have been described in details in various patents and patent application publications.

As disclosed herein, the compound from Tables 2-8 or an analogue thereof comprises individual stereoisomers, diasteteromers, conformational isomers as well as the racemates and pro-drugs thereof. The compound from Tables 2-8 or an analogue thereof can be used, for example, to treat RNA viral infections. For example, the compound from Tables 2-8 or an analogue thereof can be administered to a patient in need (for example, a patient suffering from, or at a risk of developing, one or more of the RNA viral infections disclosed herein) at a daily dosage in the range of about 0.01 to 9000 mg administered orally, for an average adult human. It is recognized by those of skill in the art that the exact dosage may be adjusted depending on the severity of symptoms, body weight of the individual and/or other clinical circumstances existing in a given individual. Moreover, it is also recognized that dosage may be adjusted when the compound from Tables 2-8 is used in combination with other pharmacologically active substances.

To prepare the pharmaceutical compositions of the present disclosure, the compounds from Tables 2-8 or analogues of compounds from Tables 2-8 can be intimately admixed with a pharmaceutically acceptable vehicle carrier according to conventional pharmaceutical compounding techniques, which may take a wide variety of forms depending on the form of preparation desired for administration (e.g., oral, transdermal, transmucosal, buccal, sublingual, transdermal, inhalation, nasal, rectal, vaginal, parenteral). In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules and tablets, suitable carriers and additives include starches, sugars, diluents, granulating agents, lubricants, binders, disintegrating agents and the like. Because of their ease in administration, tablets and capsules represent an advantageous oral dosage unit form, in which case solid pharmaceutical carriers are obviously employed. If desired, tablets may be sugar coated or enteric coated by standard techniques.

In addition, various controlled-release delivery methods, well known to those skilled in the art may be employed to improve bioavailability, reduce side effects, or transdermal delivery may be facilitated by various permeability enhancers or devises. Suppositories may be prepared, in which case cocoa butter could be used as the carrier. For parenterals, the carrier will usually comprise sterile water, though other ingredients, for example, for purposes such as aiding solubility or for preservation, may be included. Injectable suspensions may also be prepared in which case appropriate liquid carriers, suspending agents and the like may be employed. Inhalable formulations and aerosols, topical formulations, nanoparticle and microparticle formulations and bioerodible and non-bioerodible formulations may also be prepared.

Included within the scope of the present disclosure are the various individual anomers, diastereomers and enantiomers as well as mixtures thereof. Such compounds are included within the definition from Tables 2-8. For example, the selective use of a particular enantiomer (e.g. R or S) of compounds from Tables 2-8 to achieve a desired therapeutic effect is contemplated within the scope of the present disclosure since various enantiomers may have differential affinities for the host factor related to RNA viral replication. Also contemplated herein is the selective combination of various individual isomers, such as enantiomers in specific ratios (e.g. 3R:1S) to achieve a therapeutic effect. In addition, the compounds disclosed herein also include any pharmaceutically acceptable salts, for example: alkali metal salts, such as sodium and potassium; ammonium salts; monoalkylammonium salts; dialkylammonium salts; trialkylammonium salts; tetraalkylammonium salts; and tromethamine salts. Hydrates and other solvates of the compound of Tables 2-8 are included within the scope of the present disclosure.

Pharmaceutically acceptable salts of the compounds from Tables 2-8 or analogues thereof can be prepared by reacting the derivatives from Tables 2-8 with the appropriate base and recovering the salt. In some embodiments, a compound from Tables 2-8 or an analogues thereof is administered to the subject in a dosage of about 5-25 mg twice daily, or about 50 mg two or three times daily, or 100 mg once, twice or three times daily.

Also included within the scope of the present disclosure are various pro-drugs that may be converted by various physiologic processes into the active drug substance or which otherwise improves the bioavailability and/or pharmacological characteristics of the compounds disclosed herein. It is known to those of skill in the art that such pro-dugs may be created by creating derivatives of the compound from Tables 2-8 which may be changed by normal physiologic and/or metabolic processes occurring with the individual into the pharmacologically active molecules from Tables 2-8 or by combining the compound from Tables 2-8 with another molecule or promoiety so as to enhance or control for example; absorption, distribution, metabolism and/or excretion in an individual.

The present disclosure also encompasses prodrugs of the compounds disclosed herein, which on administration undergo chemical conversion by metabolic processes before becoming active pharmacological substances. In general, such prodrugs will be functional derivatives of the present compounds, which are readily convertible in vivo into the required compound from Tables 2-8. Prodrugs are any covalently bonded compounds, which release the active parent drug from Tables 2-8 in vivo. In cases in which compounds have unsaturated carbon-carbon double bonds, both the cis (Z) and trans (E) isomers are within the scope of the present disclosure. In cases wherein compounds may exist in tautomeric forms, such as ketoenol tautomers, each tautomeric form is contemplated as being included within the present disclosure whether existing in equilibrium or predominantly in one form. Conventional procedures for the selection and preparation of suitable prodrug derivatives are described, for example, in “Design of Prodrugs”, ed. H. Bundgaard, Elsevier, 1985. Prodrug designs are generally discussed in Hardma et al. (eds.), Goodman and Gilman's The Pharmacological Basis of Therapeutics, 9th ed., pages 11-16 (1996). A further thorough study of prodrug design is presented in Higuchi et al., Prodrugs as Novel Delivery Systems, vol. 14, ASCD Symposium Series, and in Roche (ed.), Bioreversible Carriers in Drug Design, American Pharmaceutical Association and Pergamon Press (1987).

The compounds from Tables 2-8 can be linked, coupled or otherwise attached to another molecule which would facilitate the transport of the compounds or derivatives across cellular or tissue barriers. For example, gastrointestinal absorption can be enhanced by coupling, linking or attaching to another molecule such as a bile acid derivative or analogues to exploit the intestinal bile acid uptake pathway so as to enhance the intestinal absorption. Examples of such conjugations of a specific drug molecule with a carrier molecule, for example a bile acid, are well known to those familiar with the art. For example, Kramer (Biochim. Biophys. Acta. 1227: 137-154, 1994b) describes the conjugation of bile acids with cholesterol lowering drugs (i.e. HMG-CoA reductase inhibitors) for example lovastatin to improve gastrointestinal absorption and to facilitate more specific target organ drug delivery.

In addition, the compounds from Tables 2-8 or analogues thereof can be linked, coupled or otherwise attached to molecules which improve penetration of the blood brain barrier. For example, coupling, linking or attaching the compounds or derivatives to an essential fatty acid or vitamin to improve penetration into the central nervous system. Such techniques and a large range of molecules and promoieties which can achieve these effects are well known to those skilled in the art of pharmaceutical science. Methods to produce prodrugs using choline derivatives are described in US Patent Application published as US2001007865. The specific examples noted in the foregoing examples are provided for illustrative purposes and are not meant in any way to limit the scope contemplated herein.

The compounds contemplated in the scope of the present disclosure may be used in conjunction with one or more other therapeutic agents (e.g., drug compounds) and used according to the methods of the present disclosure, for example the therapeutic agents have a use that is also effective in treating RNA viral infection and/or co-morbid conditions.

When administered, the pharmaceutical composition comprising one or more of the compounds disclosed herein are applied in pharmaceutically acceptable amounts and in pharmaceutically acceptable compositions. Such preparations may routinely contain salts, buffering agents, preservatives, compatible carriers, and optionally other therapeutic ingredients. When used in medicine the salts should be pharmaceutically acceptable, but non-pharmaceutically acceptable salts may conveniently be used to prepare pharmaceutically acceptable salts thereof and are not excluded herein. Such pharmacologically and pharmaceutically acceptable salts include, but are not limited to, those prepared from the following acids: hydrochloric, hydrobromic, sulfuric, nitric, phosphoric, maleic, acetic, salicylic, p-toluene sulfonic, tartaric, citric, methane sulfonic, formic, malonic, succinic, naphthalene-2-sulfonic, and benzene sulfonic. Also, pharmaceutically acceptable salts can be prepared as alkaline metal or alkaline earth salts, such as sodium, potassium or calcium salts. Suitable buffering agents include: acetic acid and a salt (1-2% W/V); citric acid and a salt (1-3% W/V); boric acid and a salt (0.5-2.5% W/V); and phosphoric acid and a salt (0.8-2% W/V). Suitable preservatives include benzalkonium chloride (0.003-0.03% W/V); chlorobutanol (0.3-0.9% W/V); parabens (0.01-0.25% W/V) and thimerosal (0.004-0.02% W/V).

The compound from Tables 2-8 and analogues thereof are preferred to be administered in safe and effective amounts. An effective amount means that amount necessary to delay; the onset, inhibit the progression, halt altogether the onset or progression of, or to reduce the clinical manifestations or symptoms of the particular condition being treated. In general, an effective amount for treating an RNA viral infection will be that amount necessary to inhibit the symptoms of the particular RNA viral infection in situ in a particular individual. When administered to an individual, effective amounts will depend, of course, on the particular condition being treated; the severity of the condition; individual patient parameters including age, physical condition, size and weight; concurrent treatment; frequency of treatment; and the mode of administration. These factors are well known to those of ordinary skill in the art and can be addressed with no more than routine experimentation. It is preferred generally that a minimum dose be used, that is, the lowest safe dosage that provides appropriate relief of symptoms.

Dosage may be adjusted appropriately to achieve desired drug levels, locally or systemically. Generally, daily doses of active compounds will be from about 0.001 mg/kg per day to 200 mg/kg per day. However, it is recognized that these are general ranges and the actual dose used as contemplated in a given individual may less or greater than this dosage range. In the event that the response in an individual subject is insufficient at such doses, even higher doses (or effective higher doses by a different, more localized delivery route) may be employed to the extent that patient tolerance permits.

A variety of administration routes can be suitable to the methods and compositions disclosed herein. The particular administration route selected can depend upon the particular drug selected, the severity of the disease state(s) being treated and the dosage required for therapeutic efficacy. The methods may be practiced using any mode of administration that is medically acceptable, meaning any mode that produces effective levels of the active compounds without causing clinically unacceptable adverse effects and multiple doses over a given period of time are also contemplated. Such modes of administration include oral, rectal, sublingual, transmucosal, buccal, inhalation, rectal, vaginal, parenteral topical, nasal, transdermal or parenteral routes. The term “parenteral” includes subcutaneous, intravenous, intramuscular, or infusion. Depot intramuscular injections suitably prepared may also be used for administration within the scope of the present disclosure.

The compositions may be conveniently presented in unit dosage form and may be prepared by any of the methods well known in the art of pharmacy. In general, the compositions are prepared by uniformly and intimately bringing the compounds into association with a liquid carrier, a finely divided solid carrier, or both, and then, if necessary, shaping the product.

Compositions suitable for oral administration may be presented as discrete units such as capsules, cachets, tablets, or lozenges, each containing a predetermined amount of the active compound. Other compositions include suspensions in aqueous liquors or non-aqueous liquids such as; a syrup, an elixir, or an emulsion.

Other delivery systems can include time-release, delayed release, sustained release or targeted release delivery systems. Such systems can avoid repeated administrations of the active compounds, increasing convenience to the subject and the physician or target release of the active compound to the tissue of interest. Many types of release delivery systems are available and known to those of ordinary skill in the art. They include polymer based systems such as polylactic and polyglycolic acid, polyanhydrides and polycaprolactone; nonpolymer systems that are lipids including sterols such as cholesterol, cholesterol esters and fatty acids or neutral fats such as mono-, di and triglycerides; hydrogel release systems; silastic systems; peptide based systems; wax coatings, compressed tablets using conventional binders and excipients, partially fused implants and the like. In addition, a pump-based hardware delivery system can be used, some of which are adapted for implantation, others of which are adapted for inhalation administration by nose or mouth.

Long-term sustained release devices, pharmaceutical compositions or molecular derivatives also may be used with the compounds described herein. “Long-term” release, as used herein, means that the drug delivery devise is constructed and arranged to deliver therapeutic levels of the active ingredient for at least 2 days, and preferably as long as 60 days. Long-term sustained release devices such as patches, implants and suppositories are well known to those of ordinary skill in the art and include some of the release systems described above. It is also contemplated by the inventors that the compounds described by the inventors may be formulated in such ways as to achieve various plasma profiles of the compounds in given individuals so as to maintain certain effective profiles of given plasma levels over a period of time. Such formulation strategies are well known to those skilled in the art and may for example include special coatings on tablets or granules containing the compounds disclosed herein either alone or in combination with other pharmacologically active substances. All such formulations are contemplated with the scope of the present disclosure.

### Methods for Treating Viral Infection

Disclosed herein include methods for preventing, delaying the onset of, or treating an infection or a disease caused by a RNA virus. In some embodiments, the method comprises administering to a subject in need thereof a composition comprising one or more compounds selected from the compounds listed in Tables 2-8, or a pharmaceutically acceptable salt, ester, solvate, stereoisomer, tautomer, or prodrug thereof, thereby preventing, delaying the onset of, or treating the infection or the disease.

Disclosed herein include methods for preventing, delaying the onset of, or treating an inflammatory effect of an infection or a disease caused by a RNA virus. In some embodiments, the method comprises administering to a subject in need thereof a composition comprising one or more compounds selected from the compounds listed in Tables 2-8, or a pharmaceutically acceptable salt, ester, solvate, stereoisomer, tautomer, or prodrug thereof, thereby preventing, delaying the onset of, or treating the inflammatory effect.

The inflammatory effect can comprise respiratory failure, a sequela of respiratory failure, acute lung injury, or acute respiratory distress syndrome. The sequela of respiratory failure can comprise multi-organ failure. As used herein, the terms “inflammation” and “inflammatory response” shall be given their ordinary meaning, and also include immune-related responses and/or allergic reactions to a physical, chemical, or biological stimulus. Measuring inflammation (e.g. lung inflammation) can comprise measuring the level of a pro-inflammatory cytokine, an anti-inflammatory cytokine, or a combination of pro-inflammatory cytokines and anti-inflammatory cytokines. Inflammation (e.g. lung inflammation) can comprise mast cell degranulation, plasma extravasation, and bronchoconstriction. Administering the composition can result in an at least, or at least about, 2% (e.g., 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 75%, 100%, 150%, 200%, 250%, 500%, 1000%, or higher and overlapping ranges therein) reduction of one or more of mast cell degranulation, plasma extravasation, and bronchoconstriction. In some embodiments of the methods and compositions provided herein, lymphopenia and/or mononuclear cell infiltration in the lungs is reduced by at least, or at least about, 2% (e.g., 2%, 3%, 4%, 5%, 6%, 7%, 8%, 9%1, 10%, 1%, %12%, 13%, 14%, 15%, 16%, 17%, 18%, 19%, 20%, 25%, 30%, 40%, 50%, 75%, 100%, 150%, 200%, 250%, 500%, 1000%, or higher and overlapping ranges therein).

A pro-inflammatory cytokine or a pro-inflammatory mediator can be an immuno-regulatory cytokine that favor inflammation. Pro-inflammatory cytokines that are generally responsible for early immune responses include IL-1, IL-6, and TNF-α. IL-1, IL-6, and TNF-α are also considered endogenous pyrogens as they contribute to increasing body temperature. Other examples of pro-inflammatory cytokines or pro-inflammatory mediators include IL-8, IL-11, IL-12, IL-18, GM-CSF, IFN-γ, TGF-β, leukemia inhibitory factors (LIF), oncostatin M (OSM), and a variety of chemokines that attract inflammatory cells. A pro-inflammatory cytokine generally up-regulates or increases the synthesis of secondary pro-inflammatory mediators and other pro-inflammatory cytokines by immune cells. In addition, pro-inflammatory cytokines can stimulate production of acute phase proteins that mediate inflammation and attract inflammatory cells. The method can comprise an at least, or at least about, 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) reduction in the level of one or more of interferon-γ (IFNγ), IL-1, IL-6, transforming growth factor-α (TGFα), transforming growth factor-β (TGFβ), CCL2, CXCL10, IL-11, IL-12, IL-18, GM-CSF, CXCL9 and IL-8 in the subject. The compositions and methods provided herein can reduce the production and/or amount of a pro-inflammatory cytokine and/or a pro-inflammatory mediator in the lung and/or serum by at least, or at least about, 2% (e.g., 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 75%, 100%, 150%, 200%, 250%, 500%, 1000%, or higher and overlapping ranges therein) compared to if the methods and compositions are not used.

The composition can comprise a therapeutically or prophylactically effective amount of one or more compounds selected from the compounds listed in Tables 2-8 (or a pharmaceutically acceptable salt, ester, solvate, stereoisomer, tautomer, or prodrug thereof). The subject in need can be a subject that can be suffering from the infection or the disease, or a subject that can be at a risk for the infection or the disease. The infection or the disease can be in the respiratory tract of the subject. The subject can have been exposed to the RNA virus, can be suspected to have been exposed to the RNA virus, or can be at a risk of being exposed to the RNA virus. The subject can be a mammal. The subject can be a human.

In some embodiments, the RNA virus can be a double-stranded RNA virus. The RNA virus can be a positive-sense single-stranded RNA virus. The positive-sense single-stranded RNA virus can be a coronavirus. The coronavirus can be an alpha coronavirus, a beta coronavirus, a gamma coronavirus, or a delta coronavirus. The coronavirus can be Middle East respiratory coronavirus (MERS-CoV), severe acute respiratory syndrome coronavirus (SARS-CoV), or SARS-CoV-2. The infection or a disease caused by the RNA virus can be common cold, influenza, SARS, coronaviruses, COVID-19, hepatitis C, hepatitis E, West Nile fever, Ebola virus disease, rabies, polio, or measles.

The method can comprise administering to the subject one or more additional antiviral agents. At least one of the one or more additional antiviral agents can be co-administered to the subject with the composition. At least one of the one or more additional antiviral agents can be administered to the subject before the administration of the composition, after the administration of the composition, or both. The composition can comprise one or more additional therapeutic agents. The one or more additional therapeutic agents comprise one or more antiviral agents. The antiviral agent can be selected from the group consisting of a nucleoside or a non-nucleoside analogue reverse-transcriptase inhibitor, a nucleotide analogue reverse-transcriptase inhibitor, a NS3/4A serine protease inhibitor, a NS5B polymerase inhibitor, and interferon alpha.

The composition can be a pharmaceutical composition comprising one or more compounds selected from the compounds listed in Tables 2-8 (or a pharmaceutically acceptable salt, ester, solvate, stereoisomer, tautomer, or prodrug thereof) and one or more pharmaceutically acceptable excipients. The composition can be administered to the subject by intravenous administration, nasal administration, pulmonary administration, oral administration, parenteral administration, or nebulization. The composition can be aspirated into at least one lung of the subject. The composition can be in the form of powder, pill, tablet, microtablet, pellet, micropellet, capsule, capsule containing microtablets, liquid, aerosols, or nanoparticles. The composition can be in a formulation for administration to the lungs. One or more compounds selected from the compounds listed in Tables 2-8 (or a pharmaceutically acceptable salt, ester, solvate, stereoisomer, tautomer, or prodrug thereof) can also be used prophylactically for preventing, delaying the onset of, or treating an infection or a disease or inflammation caused by a RNA virus. The prophylactically effective amount of one or more compounds selected from the compounds listed in Tables 2-8 (or a pharmaceutically acceptable salt, ester, solvate, stereoisomer, tautomer, or prodrug thereof) can be any therapeutically effective amount described herein.

One or more compounds selected from the compounds listed in Tables 2-8 (or a pharmaceutically acceptable salt, ester, solvate, stereoisomer, tautomer, or prodrug thereof) can be administered via any suitable route. Potential routes of administration of one or more compounds selected from the compounds listed in Tables 2-8 (or a pharmaceutically acceptable salt, ester, solvate, stereoisomer, tautomer, or prodrug thereof) include without limitation oral, parenteral (including intramuscular, subcutaneous, intradermal, intravascular, intravenous, intraarterial, intramedullary and intrathecal), intracavitary, intraperitoneal, and topical (including dermal/epicutaneous, transdermal, mucosal, transmucosal, intranasal [e.g., by nasal spray or drop], intraocular [e.g., by eye drop], pulmonary [e.g., by oral or nasal inhalation], buccal, sublingual, rectal and vaginal). In certain embodiments, one or more compounds selected from the compounds listed in Tables 2-8 (or a pharmaceutically acceptable salt, ester, solvate, stereoisomer, tautomer, or prodrug thereof) is administered orally (e.g., as a capsule or tablet, optionally with an enteric coating). In other embodiments, one or more compounds selected from the compounds listed in Tables 2-8 (or a pharmaceutically acceptable salt, ester, solvate, stereoisomer, tautomer, or prodrug thereof) is administered parenterally (e.g., intravenously, subcutaneously or intradermally). In further embodiments, one or more compounds selected from the compounds listed in Tables 2-8 (or a pharmaceutically acceptable salt, ester, solvate, stereoisomer, tautomer, or prodrug thereof) is administered topically (e.g., dermally/epicutaneously, transdermally, mucosally, transmucosally, buccally or sublingually).

In additional embodiments, one or more compounds selected from the compounds listed in Tables 2-8 (or a pharmaceutically acceptable salt, ester, solvate, stereoisomer, tautomer, or prodrug thereof) is administered without food. In some embodiments, one or more compounds selected from the compounds listed in Tables 2-8 (or a pharmaceutically acceptable salt, ester, solvate, stereoisomer, tautomer, or prodrug thereof) is administered at least about 1 or 2 hours before or after a meal. In certain embodiments, one or more compounds selected from the compounds listed in Tables 2-8 (or a pharmaceutically acceptable salt, ester, solvate, stereoisomer, tautomer, or prodrug thereof) is administered at least about 2 hours after an evening meal. One or more compounds selected from the compounds listed in Tables 2-8 (or a pharmaceutically acceptable salt, ester, solvate, stereoisomer, tautomer, or prodrug thereof) can also be taken substantially concurrently with food (e.g., within about 0.5, 1 or 2 hours before or after a meal, or with a meal).

The composition can be administered to the subject once, twice, or three times a day. The composition can be administered to the subject once every day, every two days, or every three days. The composition can be administered to the subject over the course of at least two weeks, at least three weeks, at least four weeks, or at least five weeks. The therapeutically effective amount and the frequency of administration of, and the length of treatment with one or more compounds selected from the compounds listed in Tables 2-8 (or a pharmaceutically acceptable salt, ester, solvate, stereoisomer, tautomer, or prodrug thereof) may depend on various factors, including the nature and the severity of the lung inflammation and/or infection/disease, the potency of the compound, the mode of administration, the age, the body weight, the general health, the gender and the diet of the subject, and the response of the subject to the treatment, and can be determined by the treating physician. In some embodiments, one or more compounds selected from the compounds listed in Tables 2-8 (or a pharmaceutically acceptable salt, ester, solvate, stereoisomer, tautomer, or prodrug thereof) is administered under a chronic dosing regimen. In certain embodiments, a therapeutically effective amount of one or more compounds selected from the compounds listed in Tables 2-8 (or a pharmaceutically acceptable salt, ester, solvate, stereoisomer, tautomer, or prodrug thereof) is administered over a period of at least about 6 weeks, 2 months, 10 weeks, 3 months, 4 months, 5 months, 6 months, 1 year, 1.5 years, 2 years, 3 years or longer (e.g., at least about 6 weeks, 2 months, 3 months or 6 months).

One or more compounds selected from the compounds listed in Tables 2-8 (or a pharmaceutically acceptable salt, ester, solvate, stereoisomer, tautomer, or prodrug thereof) can also be used prophylactically to for preventing, delaying the onset of, or treating an infection or a disease or inflammation caused by a RNA virus. The prophylactically effective amount of one or more compounds selected from the compounds listed in Tables 2-8 (or a pharmaceutically acceptable salt, ester, solvate, stereoisomer, tautomer, or prodrug thereof) can be any therapeutically effective amount described herein.

Administrating the composition can result in reduction of the viral titer of the RNA virus in the subject as compared to that in the subject before administration of the composition. The method can comprise determining global virus distribution in the lungs of the subject. The method can comprise measuring the viral titer of the RNA virus in the subject before administering the composition to the subject, after administering the composition to the subject, or both. The viral titer can be lung bulk virus titer.

The method can comprise measuring a neutrophil density within the lungs of the subject. Administering the composition can result in reduction of the neutrophil density within the lungs of the subject as compared to that in the subject before administration of the composition. Administering the composition can result in an at least, or at least about, 2% (e.g., 2%, 3%, 4%, 5%, 6%, 7%, 8%, 9%, 10%, 11%, 12%, 13%, 14%, 15%, 16%, 17%, 1%, %19%, 20%, 25%, 30%, 40%, 50%, 75%, 100%, 150%, 200%, 250%, 500%, 1000%, or higher and overlapping ranges therein) reduction of the neutrophil density within the lungs of the subject as compared to that in the subject before administration of the composition.

The method can comprise measuring a total necrotized cell count within the lungs of the subject. Administering the composition can result in reduction of the total necrotized cell count in the subject as compared to that in the subject before administration of the composition. The method can comprise measuring a total protein level within the lungs of the subject. Administering the composition can result in reduction of the total protein level within the lungs of the subject as compared to that in the subject before administration of the composition. In some embodiments, administering the composition results in an at least, or at least about, 2% (e.g., 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 75%, 100%, 150%, 200%, 250%, 500%, 1000%, or higher and overlapping ranges therein) reduction of the total protein level within the lungs of the subject as compared to that in the subject before administration of the composition.

Disclosed herein include methods for imparting resistance to an RNA virus to a cell(s). In some embodiments, the method comprises: contacting the cell(s) with a composition comprising a compound or a pharmaceutically acceptable salt, ester, solvate, stereoisomer, tautomer, or prodrug thereof, thereby imparting resistance to the RNA virus to the cell(s), wherein the compound is selected from the compounds listed in Tables 2-8. The method can comprise: contacting a plurality of cells with the composition. The method can comprise: determining the infection rate of the plurality of cells after being contacted with the composition. In some embodiments, contacting the cell with the composition is in a subject. In some embodiments, contacting the cell with the composition occurs in vitro, ex vivo, and/or in vivo. In some embodiments, the cell expresses angiotensin-converting enzyme 2 (ACE2). The cell can be a lung cell, an enterocyte, an endothelial cell, an epithelial cell, a kidney cell, an arterial smooth muscle cell, a cell of the respiratory tract, or any combination thereof. The cell can be the cell of a subject. The cell can be in a subject.

Disclosed herein include methods for treating or preventing infection of a cell by a RNA virus. Disclosed herein include methods for inhibiting infection of a cell by a RNA virus. In some embodiments, said methods comprises contacting the cell(s) with a composition comprising a compound or a pharmaceutically acceptable salt, ester, solvate, stereoisomer, tautomer, or prodrug thereof, wherein the compound is selected from the compounds listed in Tables 2-8.

Contacting cell(s) with the compositions provided herein (e.g., comprising one or more compounds selected from the compounds listed in Tables 2-8, or a pharmaceutically acceptable salt, ester, solvate, stereoisomer, tautomer, or prodrug thereof) can impart resistance to an RNA virus, reduce, inhibit, or prevent infection by an RNA virus, and/or treat an infection caused by an RNA virus. Contacting cell(s) with the compositions provided herein can reduce the rate of infection by an RNA virus by at least about 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 1000-fold, 10000-fold, or a number or a range between any of these values) as compared to cell(s) not contacted with said compositions. Cell(s) contacted with the compositions provided herein can be at least about 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 1000-fold, 10000-fold, or a number or a range between any of these values) more resistant to an RNA virus as compared to cell(s) not contacted with said compositions. Contacting cell(s) with the compositions provided herein can reduce RNA virus replication by at least about 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 1000-fold, 10000-fold, or a number or a range between any of these values) as compared to cell(s) not contacted with said compositions. The compositions provided herein can have no cytotoxicity or minimal cytotoxicity. The compositions provided herein can be non-toxic at a concentration of about 10, 9, 8, 7, 6, 5, 4, 3, 2, 1, 0.1, 0.5, 0.01, 0.005, 0.001, 0.0005, 0.0001, or a number or a range between any of these values, μM. The compositions provided herein can therapeutically effective at a concentration of about 10, 9, 8, 7, 6, 5, 4, 3, 2, 1, 0.1, 0.5, 0.01, 0.005, 0.001, 0.0005, 0.0001, or a number or a range between any of these values, μM. Contacting cells with the compositions provided herein can reduce normalized cell number by less than about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 70%, 80%, 90%, or a number or a range between any of these values.

### Kits and Compositions

Disclosed herein include kits comprising one or more compounds selected from the compounds listed in Tables 2-8, or a pharmaceutically acceptable salt, ester, solvate, stereoisomer, tautomer, or prodrug thereof, and a label indicating that the kit is for preventing, delaying the onset of, or treating an infection or a disease caused by a RNA virus.

Disclosed herein include kits comprising one or more compounds selected from the compounds listed in Tables 2-8, or a pharmaceutically acceptable salt, ester, solvate, stereoisomer, tautomer, or prodrug thereof, and a label indicating that the kit is for preventing, delaying the onset of, or treating an inflammatory effect of an infection or a disease caused by a RNA virus.

In some embodiments, the label indicates that the kit is for prophylaxis administration. In some embodiments, the label indicates that the kit is for low-risk patients, optionally low-risk patients exposed to an RNA virus or suspected of being exposed to an RNA virus. In some embodiments, the label indicates that the kit is for high-risk and/or severe disease patients post-infection with a RNA virus. In some embodiments, the label indicates one or more compounds selected from the compounds listed in Tables 2-8 (or the pharmaceutically acceptable salt, ester, solvate, stereoisomer, tautomer, or prodrug thereof) is administered at a daily dose of at least about 600 mg, 620 mg, 640 mg, 660 mg, 680 mg, 700 mg, 720 mg, 740 mg, 760 mg, 780 mg, 800 mg, 820 mg, 840 mg, 860 mg, 880 mg, 900 mg, 920 mg, 940 mg, 960 mg, 980 mg, 1000 mg, 1020 mg, 1040 mg, 1060 mg, 1080 mg, 1100 mg, 1120 mg, 1140 mg, 1160 mg, 1180 mg, 1200 mg, 1220 mg, 1240 mg, 1260 mg, 1280 mg, 1300 mg, 1320 mg, 1340 mg, 1360 mg, 1380 mg, 1400 mg, 1420 mg, 1440 mg, 1460 mg, 1480 mg, 1500 mg, 1520 mg, 1540 mg, 1560 mg, 1580 mg, 1600 mg, 1620 mg, 1640 mg, 1660 mg, 1680 mg, 1700 mg, 1720 mg, 1740 mg, 1760 mg, 1780 mg, 1800 mg, 1820 mg, 1840 mg, 1860 mg, 1880 mg, 1900 mg, 1920 mg, 1940 mg, 1960 mg, 1980 mg, 2000 mg, 2020 mg, 2040 mg, 2060 mg, 2080 mg, 2100 mg, 2120 mg, 2140 mg, 2160 mg, 2180 mg, 2200 mg, 2220 mg, 2240 mg, 2260 mg, 2280 mg, 2300 mg, 2320 mg, 2340 mg, 2360 mg, 2380 mg, 2400 mg, 2420 mg, 2440 mg, 2460 mg, 2480 mg, or 2500 mg, optionally the administering comprises once daily or twice daily oral administration.

The RNA virus can be a coronavirus. The coronavirus can be Middle East respiratory coronavirus (MERS-CoV), severe acute respiratory syndrome coronavirus (SARS-CoV), or SARS-CoV-2.

Disclosed herein include compositions comprising one or more compounds selected from the compounds listed in Tables 2-8, or a pharmaceutically acceptable salt, ester, solvate, stereoisomer, tautomer, or prodrug thereof, for use in preventing, delaying the onset of, or treating an infection or a disease caused by a RNA virus.

Disclosed herein include compositions comprising one or more compounds selected from the compounds listed in Tables 2-8, or a pharmaceutically acceptable salt, ester, solvate, stereoisomer, tautomer, or prodrug thereof, for use in preventing, delaying the onset of, or treating an inflammatory effect of an infection or a disease caused by a RNA virus.

The inflammatory effect can comprise respiratory failure, a sequela of respiratory failure, acute lung injury, or acute respiratory distress syndrome. The sequela of respiratory failure can comprise multi-organ failure. The composition can comprise a therapeutically or prophylactically effective amount of one or more compounds selected from the compounds listed in Tables 2-8 (or a pharmaceutically acceptable salt, ester, solvate, stereoisomer, tautomer, or prodrug thereof).

The therapeutically effective amount and the frequency of administration of, and the length of treatment with one or more compounds selected from the compounds listed in Tables 2-8 (or a pharmaceutically acceptable salt, ester, solvate, stereoisomer, tautomer, or prodrug thereof) may depend on various factors, including the nature and the severity of the lung inflammation and/or infection/disease, the potency of one or more compounds selected from the compounds listed in Tables 2-8 (or a pharmaceutically acceptable salt, ester, solvate, stereoisomer, tautomer, or prodrug thereof), the mode of administration, the age, the body weight, the general health, the gender and the diet of the subject, and the response of the subject to the treatment, and can be determined by the treating physician. In some embodiments, a therapeutically effective amount of one or more compounds selected from the compounds listed in Tables 2-8 (or a pharmaceutically acceptable salt, ester, solvate, stereoisomer, tautomer, or prodrug thereof) for treating or preventing lung inflammation, an infection, and/or a disease as described herein is about 0.1-200 mg, 0.1-150 mg, 0.1-100 mg, 0.1-50 mg, 0.1-30 mg, 0.5-20 mg, 0.5-10 mg or 1-10 mg (e.g., per day or per dose), or as deemed appropriate by the treating physician, which can be administered in a single dose or in divided doses. In certain embodiments, the therapeutically effective dose (e.g., per day or per dose) of one or more compounds selected from the compounds listed in Tables 2-8 (or a pharmaceutically acceptable salt, ester, solvate, stereoisomer, tautomer, or prodrug thereof) for treating or preventing lung inflammation, an infection, and/or a disease as described herein is about 0.1-1 mg (e.g., about 0.1 mg, 0.5 mg or 1 mg), about 1-5 mg (e.g., about 1 mg, 2 mg, 3 mg, 4 mg or 5 mg), about 5-10 mg (e.g., about 5 mg, 6 mg, 7 mg, 8 mg, 9 mg or 10 mg), about 10-20 mg (e.g., about 10 mg, 15 mg or 20 mg), about 20-30 mg (e.g., about 20 mg, 25 mg or 30 mg), about 30-40 mg (e.g., about 30 mg, 35 mg or 40 mg), about 40-50 mg (e.g., about 40 mg, 45 mg or 50 mg), about 50-100 mg (e.g., about 50 mg, 60 mg, 70 mg, 80 mg, 90 mg or 100 mg), about 100-150 mg (e.g., about 100 mg, 125 mg or 150 mg), about 150-200 mg (e.g., about 150 mg, 175 mg or 200 mg), about 200-300 mg (e.g., about 200 mg, 220 mg, 240 mg, 260 mg, 280 mg, or 300 mg), about 300-400 mg (e.g., about 300 mg, 320 mg, 340 mg, 360 mg, 380 mg, or 400 mg), about 400-500 mg (e.g., about 400 mg, 420 mg, 440 mg, 460 mg, 480 mg, or 500 mg), about 500-600 mg (e.g., about 500 mg, 520 mg, 540 mg, 560 mg, 580 mg, or 600 mg), or about 600-700 mg (e.g., about 600 mg, 620 mg, 640 mg, 660 mg, 680 mg, or 700 mg). In certain embodiments, the therapeutically effective dose (e.g., per day or per dose) of one or more compounds selected from the compounds listed in Tables 2-8 (or a pharmaceutically acceptable salt, ester, solvate, stereoisomer, tautomer, or prodrug thereof) for treating or preventing lung inflammation, an infection, and/or a disease as described herein is about 600 mg, 620 mg, 640 mg, 660 mg, 680 mg, 700 mg, 720 mg, 740 mg, 760 mg, 780 mg, 800 mg, 820 mg, 840 mg, 860 mg, 880 mg, 900 mg, 920 mg, 940 mg, 960 mg, 980 mg, 1000 mg, 1020 mg, 1040 mg, 1060 mg, 1080 mg, 1100 mg, 1120 mg, 1140 mg, 1160 mg, 1180 mg, 1200 mg, 1220 mg, 1240 mg, 1260 mg, 1280 mg, 1300 mg, 1320 mg, 1340 mg, 1360 mg, 1380 mg, 1400 mg, 1420 mg, 1440 mg, 1460 mg, 1480 mg, 1500 mg, 1520 mg, 1540 mg, 1560 mg, 1580 mg, 1600 mg, 1620 mg, 1640 mg, 1660 mg, 1680 mg, 1700 mg, 1720 mg, 1740 mg, 1760 mg, 1780 mg, 1800 mg, 1820 mg, 1840 mg, 1860 mg, 1880 mg, 1900 mg, 1920 mg, 1940 mg, 1960 mg, 1980 mg, 2000 mg, 2020 mg, 2040 mg, 2060 mg, 2080 mg, 2100 mg, 2120 mg, 2140 mg, 2160 mg, 2180 mg, 2200 mg, 2220 mg, 2240 mg, 2260 mg, 2280 mg, 2300 mg, 2320 mg, 2340 mg, 2360 mg, 2380 mg, 2400 mg, 2420 mg, 2440 mg, 2460 mg, 2480 mg, 2500 mg, or greater. In some embodiments, one or more compounds selected from the compounds listed in Tables 2-8 (or a pharmaceutically acceptable salt, ester, solvate, stereoisomer, tautomer, or prodrug thereof) is administered for treating or preventing lung inflammation, an infection, and/or a disease as described herein at a daily dose, weekly dose, and/or monthly dose of about 0.1-1 mg (e.g., about 0.1 mg, 0.5 mg or 1 mg), about 1-5 mg (e.g., about 1 mg, 2 mg, 3 mg, 4 mg or 5 mg), about 5-10 mg (e.g., about 5 mg, 6 mg, 7 mg, 8 mg, 9 mg or 10 mg), about 10-20 mg (e.g., about 10 mg, 15 mg or 20 mg), about 20-30 mg (e.g., about 20 mg, 25 mg or 30 mg), about 30-40 mg (e.g., about 30 mg, 35 mg or 40 mg), about 40-50 mg (e.g., about 40 mg, 45 mg or 50 mg), about 50-100 mg (e.g., about 50 mg, 60 mg, 70 mg, 80 mg, 90 mg or 100 mg), about 100-150 mg (e.g., about 100 mg, 125 mg or 150 mg), about 150-200 mg (e.g., about 150 mg, 175 mg or 200 mg), about 200-300 mg (e.g., about 200 mg, 220 mg, 240 mg, 260 mg, 280 mg, or 300 mg), about 300-400 mg (e.g., about 300 mg, 320 mg, 340 mg, 360 mg, 380 mg, or 400 mg), about 400-500 mg (e.g., about 400 mg, 420 mg, 440 mg, 460 mg, 480 mg, or 500 mg), about 500-600 mg (e.g., about 500 mg, 520 mg, 540 mg, 560 mg, 580 mg, or 600 mg), or about 600-700 mg (e.g., about 600 mg, 620 mg, 640 mg, 660 mg, 680 mg, or 700 mg). In some embodiments, one or more compounds selected from the compounds listed in Tables 2-8 (or a pharmaceutically acceptable salt, ester, solvate, stereoisomer, tautomer, or prodrug thereof) is administered for treating or preventing lung inflammation, an infection, and/or a disease as described herein at a daily dose, weekly dose, and/or monthly dose of about 600 mg, 620 mg, 640 mg, 660 mg, 680 mg, 700 mg, 720 mg, 740 mg, 760 mg, 780 mg, 800 mg, 820 mg, 840 mg, 860 mg, 880 mg, 900 mg, 920 mg, 940 mg, 960 mg, 980 mg, 1000 mg, 1020 mg, 1040 mg, 1060 mg, 1080 mg, 1100 mg, 1120 mg, 1140 mg, 1160 mg, 1180 mg, 1200 mg, 1220 mg, 1240 mg, 1260 mg, 1280 mg, 1300 mg, 1320 mg, 1340 mg, 1360 mg, 1380 mg, 1400 mg, 1420 mg, 1440 mg, 1460 mg, 1480 mg, 1500 mg, 1520 mg, 1540 mg, 1560 mg, 1580 mg, 1600 mg, 1620 mg, 1640 mg, 1660 mg, 1680 mg, 1700 mg, 1720 mg, 1740 mg, 1760 mg, 1780 mg, 1800 mg, 1820 mg, 1840 mg, 1860 mg, 1880 mg, 1900 mg, 1920 mg, 1940 mg, 1960 mg, 1980 mg, 2000 mg, 2020 mg, 2040 mg, 2060 mg, 2080 mg, 2100 mg, 2120 mg, 2140 mg, 2160 mg, 2180 mg, 2200 mg, 2220 mg, 2240 mg, 2260 mg, 2280 mg, 2300 mg, 2320 mg, 2340 mg, 2360 mg, 2380 mg, 2400 mg, 2420 mg, 2440 mg, 2460 mg, 2480 mg, 2500 mg, or greater. The daily dose, weekly dose, and/or monthly dose of one or more compounds selected from the compounds listed in Tables 2-8 (or a pharmaceutically acceptable salt, ester, solvate, stereoisomer, tautomer, or prodrug thereof) can comprise a single administration (e.g., a weekly dose can administered once per week) or multiple administrations. In some embodiments, the dosing regimen comprises administering one or more loading doses and one or more maintenance doses. The term “loading dose” shall be given its ordinary meaning, and shall also refer to a single dose or short duration regimen of a multiple doses having a dosage higher than one or more maintenance doses. A loading dose can, for example, rapidly increase the blood concentration level of one or more compounds selected from the compounds listed in Tables 2-8 (or a pharmaceutically acceptable salt, ester, solvate, stereoisomer, tautomer, or prodrug thereof). In some embodiments, the loading dose can increase the blood concentration of one or more compounds selected from the compounds listed in Tables 2-8 (or a pharmaceutically acceptable salt, ester, solvate, stereoisomer, tautomer, or prodrug thereof) to a therapeutically effective level in conjunction with a maintenance dose of the compound. The loading dose can be administered once per day, or more than once per day (e.g., up to 4 times per day). The term “maintenance dose” as used herein shall be given its ordinary meaning, and shall also refer to a dose that is serially administered (e.g., at least twice) which is intended to either slowly raise blood concentration levels of one or more compounds selected from the compounds listed in Tables 2-8 (or a pharmaceutically acceptable salt, ester, solvate, stereoisomer, tautomer, or prodrug thereof) to a therapeutically effective level, or to maintain such a therapeutically effective level. The daily dose of the maintenance dose can lower than the total daily dose of the loading dose.

The RNA virus can be a double-stranded RNA virus. The RNA virus can be a positive-sense single-stranded RNA virus. The positive-sense single-stranded RNA virus can be a coronavirus. The coronavirus can be an alpha coronavirus, a beta coronavirus, a gamma coronavirus, or a delta coronavirus. The coronavirus can be Middle East respiratory coronavirus (MERS-CoV), severe acute respiratory syndrome coronavirus (SARS-CoV), or SARS-CoV-2.

The composition can be a pharmaceutical composition comprising one or more compounds selected from the compounds listed in Tables 2-8 (or a pharmaceutically acceptable salt, ester, solvate, stereoisomer, tautomer, or prodrug thereof) and one or more pharmaceutically acceptable excipients. The composition can comprise one or more additional therapeutic agents. The one or more additional therapeutic agents comprise one or more antiviral agents. The one or more antiviral agents can be selected from the group consisting of a nucleoside or a non-nucleoside analogue reverse-transcriptase inhibitor, a nucleotide analogue reverse-transcriptase inhibitor, a NS3/4A serine protease inhibitor, a NS5B polymerase inhibitor, and interferon alpha.

The composition can be in the form of powder, pill, tablet, microtablet, pellet, micropellet, capsule, capsule containing microtablets, liquid, aerosols, or nanoparticles. The composition can be in a formulation for administration to the lungs. As disclosed herein, one or more compounds selected from the compounds listed in Tables 2-8 (or a pharmaceutically acceptable salt, ester, solvate, stereoisomer, tautomer, or prodrug thereof) can be formulated for administration in a pharmaceutical composition comprising a physiologically acceptable surface active agents, carriers, diluents, excipients, smoothing agents, suspension agents, film forming substances, coating assistants, or a combination thereof. In some embodiments, one or more compounds selected from the compounds listed in Tables 2-8 (or a pharmaceutically acceptable salt, ester, solvate, stereoisomer, tautomer, or prodrug thereof) is formulated for administration with a pharmaceutically acceptable carrier or diluent. One or more compounds selected from the compounds listed in Tables 2-8 (or a pharmaceutically acceptable salt, ester, solvate, stereoisomer, tautomer, or prodrug thereof) can be formulated as a medicament with a standard pharmaceutically acceptable carrier(s) and/or excipient(s) as is routine in the pharmaceutical art. The exact nature of the formulation will depend upon several factors including the desired route of administration. One or more compounds selected from the compounds listed in Tables 2-8 (or a pharmaceutically acceptable salt, ester, solvate, stereoisomer, tautomer, or prodrug thereof) can be formulated for oral, intravenous, intragastric, intravascular or intraperitoneal administration. Standard pharmaceutical formulation techniques may be used, such as those disclosed in Remington's The Science and Practice of Pharmacy, 21st Ed., Lippincott Williams & Wilkins (2005), incorporated herein by reference in its entirety.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, glucose and sucrose: starches, such as corn starch and potato starch; cellulose and its derivatives, such as sodium carboxymethyl cellulose, powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroraa; polyols such as propylene glycol, glycerine, sorbitol, mannitol, and polyethylene glycol; aiginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with the subject therapeutic agent is basically determined by the way the composition is to be administered.

The compositions described herein are preferably provided in unit dosage form. As used herein, a “unit dosage form” is a composition containing an amount of one or more compounds selected from the compounds listed in Tables 2-8 (or a pharmaceutically acceptable salt, ester, solvate, stereoisomer, tautomer, or prodrug thereof) that is suitable for administration to an animal, preferably mammal subject, in a single dose, according to good medical practice. The preparation of a single or unit dosage form however, does not imply that the dosage form is administered once per day or once per course of therapy. Such dosage forms are contemplated to be administered once, twice, thrice or more per day and may be administered as infusion over a period of time (e.g., from about 30 minutes to about 2-6 hours), or administered as a continuous infusion, and may be given more than once during a course of therapy, though a single administration is not specifically excluded. The skilled artisan will recognize that the formulation does not specifically contemplate the entire course of therapy and such decisions are left for those skilled in the art of treatment rather than formulation.

The compositions useful as described above may be in any of a variety of suitable forms for a variety of routes for administration, for example, for oral, nasal, rectal, topical (including transdermal), ocular, intracerebral, intracranial, intrathecal, intra-arterial, intravenous, intramuscular, or other parental routes of administration. The skilled artisan will appreciate that oral and nasal compositions include compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropies, surface-active agents, and encapsulating substances. Optional pharmaceutically-active materials may be included, which do not substantially interfere with the activity of one or more compounds selected from the compounds listed in Tables 2-8 (or a pharmaceutically acceptable salt, ester, solvate, stereoisomer, tautomer, or prodrug thereof). The amount of carrier employed in conjunction with one or more compounds selected from the compounds listed in Tables 2-8 (or a pharmaceutically acceptable salt, ester, solvate, stereoisomer, tautomer, or prodrug thereof) is sufficient to provide a practical quantity of material for administration per unit dose of the disclosed compositions. Techniques and compositions for making dosage forms useful in the methods described herein are described in the following references, ail incorporated by reference herein: Modern Pharmaceutics, 4th Ed., Chapters 9 and 10 (Banker & Rhodes, editors, 2002); Lieberman et αi, Pharmaceutical Dosage Forms: Tablets (1989), and Ansel, Introduction to Pharmaceutical Dosage Forms 8th Edition (2004).

Various oral dosage forms can be used, including such solid forms as tablets, capsules, and granules. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carriers suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Other compositions useful for attaining systemic delivery of the subject therapeutic agents include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

For topical use, creams, ointments, gels, solutions or suspensions, etc., containing one or more compounds selected from the compounds listed in Tables 2-8 (or a pharmaceutically acceptable salt, ester, solvate, stereoisomer, tautomer, or prodrug thereof) are employed. Topical formulations may generally be comprised of a pharmaceutical carrier, co-solvent, emulsifier, penetration enhancer, preservative system, and emollient.

For intravenous administration, one or more compounds selected from the compounds listed in Tables 2-8 (or a pharmaceutically acceptable salt, ester, solvate, stereoisomer, tautomer, or prodrug thereof) and compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-31 1 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenyl mercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of one or more compounds selected from the compounds listed in Tables 2-8 (or a pharmaceutically acceptable salt, ester, solvate, stereoisomer, tautomer, or prodrug thereof) and another agent, the combination may be provided to caregivers as a mixture, or the caregivers may mix the two agents prior to administration, or the two agents may be administered separately.

In non-human animal studies, applications of potential products are commenced at higher dosage levels, with dosage being decreased until the desired effect is no longer achieved or adverse side effects disappear. The dosage may range broadly, depending upon the desired effects and the therapeutic indication. Typically, dosages may be between about 0.1 mg/kg and 4000 mg/kg body weight, preferably between about 80 mg/kg and 1600 mg/kg body weight. Alternatively dosages may be based and calculated upon the surface area of the patient, as understood by those of skill in the art.

Depending on the severity and responsiveness of the condition to be treated, dosing can also be a single administration of a slow release composition, with course of treatment lasting from several days to several weeks or until cure is effected or diminution of the disease state is achieved. The amount of a composition to be administered will depend on many factors including the subject being treated, the severity of the affliction, the manner of administration, the judgment of the prescribing physician. One or more compounds selected from the compounds listed in Tables 2-8 (or a pharmaceutically acceptable salt, ester, solvate, stereoisomer, tautomer, or prodrug thereof) or combination of therapeutic agents (e.g., an antiviral agent provided herein in combination with one or more compounds selected from the compounds listed in Tables 2-8 (or a pharmaceutically acceptable salt, ester, solvate, stereoisomer, tautomer, or prodrug thereof)) may be administered orally or via injection at a dose from 0, 1 mg/kg to 4000 mg/kg of the patient's body weight per day. The dose range for adult humans is generally from 1 g to 100 g/day. Tablets or other forms of presentation provided in discrete units may conveniently contain an amount of one or more compounds selected from the compounds listed in Tables 2-8 (or a pharmaceutically acceptable salt, ester, solvate, stereoisomer, tautomer, or prodrug thereof) or combination of compounds disclosed herein (e.g., an antiviral agent provided herein in combination with one or more compounds selected from the compounds listed in Tables 2-8 (or a pharmaceutically acceptable salt, ester, solvate, stereoisomer, tautomer, or prodrug thereof)) which is effective at such dosage or as a multiple of the same, for instance, units containing 1 g to 60 g (for example, from about 5 g to 20 g, from about 10 g to 50 g, from about 20 g to 40 g, or from about 25 g to 35 g). The precise amount of therapeutic agent administered to a patient is the responsibility of the attendant physician. However, the dose employed can depend on a number of factors, including the age and sex of the patient, the precise disorder being treated, and its severity. Additionally, the route of administration may vary depending on the condition and its severity. A typical dose of one or more compounds selected from the compounds listed in Tables 2-8 (or a pharmaceutically acceptable salt, ester, solvate, stereoisomer, tautomer, or prodrug thereof) can be from 0.02 g to 1.25 g per kg of body weight, for example from 0.1 g to 0.5 g per kg of body weight, depending on such parameters. In some embodiments, the dosage of one or more compounds selected from the compounds listed in Tables 2-8 (or a pharmaceutically acceptable salt, ester, solvate, stereoisomer, tautomer, or prodrug thereof) can be from 1 g to 100 g, for example, from 10 g to 80 g, from 15 g to 60 g, from 20 g to 40 g, or from 25 g to 35 g. A physician will be able to determine the required dosage of one or more compounds selected from the compounds listed in Tables 2-8 (or a pharmaceutically acceptable salt, ester, solvate, stereoisomer, tautomer, or prodrug thereof) for any particular subject.

The exact formulation, route of administration and dosage for the pharmaceutical compositions comprising one or more compounds selected from the compounds listed in Tables 2-8 (or a pharmaceutically acceptable salt, ester, solvate, stereoisomer, tautomer, or prodrug thereof) or combination of therapeutic agents disclosed herein can be chosen by the individual physician in view of the patient's condition. (See, e.g., Fingl et al. 1975, in “The Pharmacological Basis of Therapeutics,” which is hereby incorporated herein by reference, with particular reference to Ch. 1). Typically, the dose range of the composition administered to the patient can be from about 0.1 to about 4000 mg/kg of the patient's body weight. The dosage may be a single one or a series of two or more given in the course of one or more days, as is needed by the patient. In instances where human dosages for therapeutic agents have been established for at least some condition, the present disclosure will use those same dosages, or dosages that are between about 0.1% and about 5000%, more preferably between about 25% and about 1000% of the established human dosage. Where no human dosage is established, as will be the case for newly-discovered pharmaceutical compounds, a suitable human dosage can be inferred from ED₅₀ or ID₅₀ values, or other appropriate values derived from in vitro or in vivo studies, as qualified by toxicity studies and efficacy studies in animals.

The attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Although the exact dosage will be determined on a drug-by-drug basis, in most cases, some generalizations regarding the dosage can be made. In cases of administration of a pharmaceutically acceptable salt, dosages may be calculated as the free base. In some embodiments, the composition is administered 1 to 4 times per day. Alternatively the compositions disclosed herein may be administered by continuous intravenous infusion, e.g., at a dose of each active ingredient up to 100 g per day. As will be understood by those of skill in the art, in certain situations it may be necessary to administer the compositions disclosed herein in amounts that exceed, or even far exceed, the above-stated, preferred dosage range in order to effectively and aggressively treat particularly aggressive diseases or infections. In some embodiments, one or more compounds selected from the compounds listed in Tables 2-8 (or a pharmaceutically acceptable salt, ester, solvate, stereoisomer, tautomer, or prodrug thereof) or combination of therapeutic agents disclosed herein will be administered for a period of continuous therapy, for example for a week or more, or for months or years.

In some embodiments, the dosing regimen of one or more compounds selected from the compounds listed in Tables 2-8 (or a pharmaceutically acceptable salt, ester, solvate, stereoisomer, tautomer, or prodrug thereof) or combination of therapeutic agents disclosed herein is administered for a period of time, which time period can be, for example, from at least about 1 week to at least about 4 weeks, from at least about 4 weeks to at least about 8 weeks, from at least about 4 weeks to at least about 12 weeks, from at least about 4 weeks to at least about 16 weeks, or longer. The dosing regimen of one or more compounds selected from the compounds listed in Tables 2-8 (or a pharmaceutically acceptable salt, ester, solvate, stereoisomer, tautomer, or prodrug thereof) or combination of therapeutic agents disclosed herein can be administered three times a day, twice a day, daily, every other day, three times a week, every other week, three times per month, once monthly, substantially continuously or continuously.

A compound disclosed herein (e.g., one or more compounds selected from the compounds listed in Tables 2-8, or a pharmaceutically acceptable salt, ester, solvate, stereoisomer, tautomer, or prodrug thereof) can be administered alone or in the form of a composition (e.g., a pharmaceutical composition). In some embodiments, a pharmaceutical composition comprises one or more compounds selected from the compounds listed in Tables 2-8 (or a pharmaceutically acceptable salt, ester, solvate, stereoisomer, tautomer, or prodrug thereof), and one or more pharmaceutically acceptable carriers or excipients. The composition can optionally contain one or more additional therapeutic agents as described herein. A pharmaceutical composition can contain a therapeutically effective amount of one or more compounds selected from the compounds listed in Tables 2-8 (or a pharmaceutically acceptable salt, ester, solvate, stereoisomer, tautomer, or prodrug thereof) and one or more pharmaceutically acceptable carriers or excipients, and can be formulated for administration to a subject for therapeutic use. For purposes of the content of a pharmaceutical composition, the terms “therapeutic agent”, “active ingredient”, “active agent” and “drug” encompass prodrugs.

A pharmaceutical composition can contain one or more compounds selected from the compounds listed in Tables 2-8 (or a pharmaceutically acceptable salt, ester, solvate, stereoisomer, tautomer, or prodrug thereof) in substantially pure form. In some embodiments, the purity of the therapeutic agent is at least about 95%, 96%, 97%, 98% or 99%. In certain embodiments, the purity of the therapeutic agent is at least about 98% or 99%. In addition, a pharmaceutical composition is substantially free of contaminants or impurities. In some embodiments, the level of contaminants or impurities other than residual solvent in a pharmaceutical composition is no more than about 5%, 4%, 3%, 2% or 1% relative to the combined weight of the intended active and inactive ingredients. In certain embodiments, the level of contaminants or impurities other than residual solvent in a pharmaceutical composition is no more than about 2% or 1% relative to the combined weight of the intended active and inactive ingredients. Pharmaceutical compositions generally are prepared according to current good manufacturing practice (GMP), as recommended or required by, e.g., the Federal Food, Drug, and Cosmetic Act § 501(a)(2)(B) and the International Conference on Harmonisation Q7 Guideline.

Pharmaceutically acceptable carriers and excipients include pharmaceutically acceptable materials, vehicles and substances. Non-limiting examples of excipients include liquid and solid fillers, diluents, binders, lubricants, glidants, solubilizers, surfactants, dispersing agents, disintegration agents, emulsifying agents, wetting agents, suspending agents, thickeners, solvents, isotonic agents, buffers, pH adjusters, stabilizers, preservatives, antioxidants, antimicrobial agents, antibacterial agents, antifungal agents, absorption-delaying agents, sweetening agents, flavoring agents, coloring agents, adjuvants, encapsulating materials and coating materials. The use of such excipients in pharmaceutical formulations is known in the art. For example, conventional vehicles and carriers include without limitation oils (e.g., vegetable oils, such as sesame oil), aqueous solvents (e.g., saline, phosphate-buffered saline [PBS] and isotonic solutions [e.g., Ringer's solution]), and solvents (e.g., dimethyl sulfoxide [DMSO] and alcohols [e.g., ethanol, glycerol and propylene glycol]). Except insofar as any conventional carrier or excipient is incompatible with the active ingredient, the disclosure encompasses the use of conventional carriers and excipients in formulations containing a therapeutic agent (e.g., one or more compounds selected from the compounds listed in Tables 2-8, or a pharmaceutically acceptable salt, ester, solvate, stereoisomer, tautomer, or prodrug thereof). See, e.g., Remington: The Science and Practice of Pharmacy, 21st Ed., Lippincott Williams & Wilkins (Philadelphia, Pennsylvania [2005]); Handbook of Pharmaceutical Excipients, 5th Ed., Rowe et al., Eds., The Pharmaceutical Press and the American Pharmaceutical Association (2005); Handbook of Pharmaceutical Additives, 3rd Ed., Ash and Ash, Eds., Gower Publishing Co. (2007); and Pharmaceutical Preformulation and Formulation, Gibson, Ed., CRC Press (Boca Raton, Florida, 2004).

Proper formulation can depend on various factors, such as the mode of administration chosen. Potential modes of administration of pharmaceutical compositions comprising one or more compounds selected from the compounds listed in Tables 2-8 (or a pharmaceutically acceptable salt, ester, solvate, stereoisomer, tautomer, or prodrug thereof) include without limitation oral, parenteral (including intramuscular, subcutaneous, intradermal, intravascular, intravenous, intraarterial, intraperitoneal, intramedullary, intrathecal and topical), intracavitary, and topical (including dermal/epicutaneous, transdermal, mucosal, transmucosal, intranasal [e.g., by nasal spray or drop], pulmonary [e.g., by oral or nasal inhalation], buccal, sublingual, rectal [e.g., by suppository], and vaginal [e.g., by suppository]).

As an example, formulations of one or more compounds selected from the compounds listed in Tables 2-8 (or a pharmaceutically acceptable salt, ester, solvate, stereoisomer, tautomer, or prodrug thereof) suitable for oral administration can be presented as, e.g., boluses; tablets, capsules, pills, cachets or lozenges; as powders or granules; as semisolids, electuaries, pastes or gels; as solutions or suspensions in an aqueous liquid or/and a non-aqueous liquid; or as oil-in-water liquid emulsions or water-in-oil liquid emulsions.

Tablets can contain one or more compounds selected from the compounds listed in Tables 2-8 (or a pharmaceutically acceptable salt, ester, solvate, stereoisomer, tautomer, or prodrug thereof) in admixture with, e.g., a filler or inert diluent (e.g., calcium carbonate, calcium phosphate, lactose, mannitol or microcrystalline cellulose), a binding agent (e.g., a starch, gelatin, acacia, alginic acid or a salt thereof, or microcrystalline cellulose), a lubricating agent (e.g., stearic acid, magnesium stearate, talc or silicon dioxide), and a disintegrating agent (e.g., crospovidone, croscarmellose sodium or colloidal silica), and optionally a surfactant (e.g., sodium lauryl sulfate). The tablets can be uncoated or can be coated with, e.g., an enteric coating that protects the active ingredient from the acidic environment of the stomach, or with a material that delays disintegration and absorption of the active ingredient in the gastrointestinal tract and thereby provides a sustained action over a longer time period. In certain embodiments, a tablet comprises one or more compounds selected from the compounds listed in Tables 2-8 (or a pharmaceutically acceptable salt, ester, solvate, stereoisomer, tautomer, or prodrug thereof), mannitol, microcrystalline cellulose, magnesium stearate, silicon dioxide, croscarmellose sodium and sodium lauryl sulfate, and optionally lactose monohydrate, and the tablet is optionally film-coated (e.g., with Opadry®).

Push-fit capsules or two-piece hard gelatin capsules can contain one or more compounds selected from the compounds listed in Tables 2-8 (or a pharmaceutically acceptable salt, ester, solvate, stereoisomer, tautomer, or prodrug thereof) in admixture with, e.g., a filler or inert solid diluent (e.g., calcium carbonate, calcium phosphate, kaolin or lactose), a binder (e.g., a starch), a glidant or lubricant (e.g., talc or magnesium stearate), and a disintegrant (e.g., crospovidone), and optionally a stabilizer or/and a preservative. For soft capsules or single-piece gelatin capsules, one or more compounds selected from the compounds listed in Tables 2-8 (or a pharmaceutically acceptable salt, ester, solvate, stereoisomer, tautomer, or prodrug thereof) can be dissolved or suspended in a suitable liquid (e.g., liquid polyethylene glycol or an oil medium, such as a fatty oil, peanut oil, olive oil or liquid paraffin), and the liquid-filled capsules can contain one or more other liquid excipients or/and semi-solid excipients, such as a stabilizer or/and an amphiphilic agent (e.g., a fatty acid ester of glycerol, propylene glycol or sorbitol).

Compositions for oral administration can also be formulated as solutions or suspensions in an aqueous liquid or/and a non-aqueous liquid, or as oil-in-water liquid emulsions or water-in-oil liquid emulsions. Dispersible powder or granules of one or more compounds selected from the compounds listed in Tables 2-8 (or a pharmaceutically acceptable salt, ester, solvate, stereoisomer, tautomer, or prodrug thereof) can be mixed with any suitable combination of an aqueous liquid, an organic solvent or/and an oil and any suitable excipients (e.g., any combination of a dispersing agent, a wetting agent, a suspending agent, an emulsifying agent or/and a preservative) to form a solution, suspension or emulsion.

In some embodiments, one or more compounds selected from the compounds listed in Tables 2-8 (or a pharmaceutically acceptable salt, ester, solvate, stereoisomer, tautomer, or prodrug thereof) is contained in an amphiphilic vehicle of a liquid or semi-solid formulation for oral administration which provides improved solubility, stability and bioavailability of the compound, as described in US 2010/0209496. The amphiphilic vehicle contains a solution, suspension, emulsion (e.g., oil-in-water emulsion) or semi-solid mixture of the compound admixed with liquid or/and semi-solid excipients which fills an encapsulated dosage form (e.g., a hard gelatin capsule or a soft gelatin capsule containing a plasticizer [e.g., glycerol or/and sorbitol]). In some embodiments, the amphiphilic vehicle comprises an amphiphilic agent selected from fatty acid esters of glycerol (glycerin), propylene glycol and sorbitol. In certain embodiments, the amphiphilic agent is selected from mono- and di-glycerides of C₈-C₁₂ saturated fatty acids. In further embodiments, the amphiphilic agent is selected from CAPMUL® MCM, CAPMUL® MCM 8, CAPMUL® MCM 10, IMWITOR® 308, ITMWITOR® 624, IMWITOR® 742, IMWITOR® 988, CAPRYOL™ PGMC, CAPRYOL™ 90, LAUROGLYCOL™ 90, CAPTEX® 200, CRILL™ 1, CRILL™ 4, PECEOL® and MAIS INE™ 35-1. In some embodiments, the amphiphilic vehicle further comprises propylene glycol, a propylene glycol-sparing agent (e.g., ethanol or/and glycerol), or an antioxidant (e.g., butylated hydroxyanisole, butylated hydroxytoluene, propyl gallate or/and sodium sulfite), or any combination thereof. In additional embodiments, the amphiphilic vehicle contains on a weight basis about 0.1-5% of the compound, about 50-90% of the amphiphilic agent, about 5-40% of propylene glycol, about 5-20% of the propylene glycol-sparing agent, and about 0.01-0.5% of the antioxidant.

One or more compounds selected from the compounds listed in Tables 2-8 (or a pharmaceutically acceptable salt, ester, solvate, stereoisomer, tautomer, or prodrug thereof) can also be formulated for parenteral administration by injection or infusion to circumvent gastrointestinal absorption and first-pass metabolism. A representative parenteral route is intravenous.

Additional advantages of intravenous administration include direct administration of a therapeutic agent into systemic circulation to achieve a rapid systemic effect, and the ability to administer the agent continuously or/and in a large volume if desired. Formulations for injection or infusion can be in the form of, e.g., solutions, suspensions or emulsions in oily or aqueous vehicles, and can contain excipients such as suspending agents, dispersing agents or/and stabilizing agents. For example, aqueous or non-aqueous (e.g., oily) sterile injection solutions can contain one or more compounds selected from the compounds listed in Tables 2-8 (or a pharmaceutically acceptable salt, ester, solvate, stereoisomer, tautomer, or prodrug thereof) along with excipients such as an antioxidant, a buffer, a bacteriostat and solutes that render the formulation isotonic with the blood of the subject. Aqueous or non-aqueous sterile suspensions can contain one or more compounds selected from the compounds listed in Tables 2-8 (or a pharmaceutically acceptable salt, ester, solvate, stereoisomer, tautomer, or prodrug thereof) along with excipients such as a suspending agent and a thickening agent, and optionally a stabilizer and an agent that increases the solubility of the compound to allow for the preparation of a more concentrated solution or suspension. As another example, a sterile aqueous solution for injection or infusion (e.g., subcutaneously or intravenously) can contain one or more compounds selected from the compounds listed in Tables 2-8 (or a pharmaceutically acceptable salt, ester, solvate, stereoisomer, tautomer, or prodrug thereof), NaCl, a buffering agent (e.g., sodium citrate), a preservative (e.g., meta-cresol), and optionally a base (e.g., NaOH) or/and an acid (e.g., HCl) to adjust pH.

For topical administration, one or more compounds selected from the compounds listed in Tables 2-8 (or a pharmaceutically acceptable salt, ester, solvate, stereoisomer, tautomer, or prodrug thereof) can be formulated as, e.g., a buccal or sublingual tablet or pill. Advantages of a buccal or sublingual tablet or pill include avoidance of first-pass metabolism and circumvention of gastrointestinal absorption. A buccal or sublingual tablet or pill can also be designed to provide faster release of the compound for more rapid uptake of it into systemic circulation. In addition to a therapeutically effective amount of one or more compounds selected from the compounds listed in Tables 2-8 (or a pharmaceutically acceptable salt, ester, solvate, stereoisomer, tautomer, or prodrug thereof), the buccal or sublingual tablet or pill can contain suitable excipients, including without limitation any combination of fillers and diluents (e.g., mannitol and sorbitol), binding agents (e.g., sodium carbonate), wetting agents (e.g., sodium carbonate), disintegrants (e.g., crospovidone and croscarmellose sodium), lubricants (e.g., silicon dioxide [including colloidal silicon dioxide] and sodium stearyl fumarate), stabilizers (e.g., sodium bicarbonate), flavoring agents (e.g., spearmint flavor), sweetening agents (e.g., sucralose), and coloring agents (e.g., yellow iron oxide).

For topical administration, one or more compounds selected from the compounds listed in Tables 2-8 (or a pharmaceutically acceptable salt, ester, solvate, stereoisomer, tautomer, or prodrug thereof) can also be formulated for intranasal administration. The nasal mucosa provides a big surface area, a porous endothelium, a highly vascular subepithelial layer and a high absorption rate, and hence allows for high bioavailability. An intranasal solution or suspension formulation can comprise one or more compounds selected from the compounds listed in Tables 2-8 (or a pharmaceutically acceptable salt, ester, solvate, stereoisomer, tautomer, or prodrug thereof) along with excipients such as a solubility enhancer (e.g., propylene glycol), a humectant (e.g., mannitol or sorbitol), a buffer and water, and optionally a preservative (e.g., benzalkonium chloride), a mucoadhesive agent (e.g., hydroxyethylcellulose) or/and a penetration enhancer. In certain embodiments, a nasal spray formulation comprises one or more compounds selected from the compounds listed in Tables 2-8 (or a pharmaceutically acceptable salt, ester, solvate, stereoisomer, tautomer, or prodrug thereof), microcrystalline cellulose, sodium carboxymethylcellulose, dextrose and water, and optionally an acid (e.g., HCl) to adjust pH. An intranasal solution or suspension formulation can be administered to the nasal cavity by any suitable means, including but not limited to a dropper, a pipette, or spray using, e.g., a metering atomizing spray pump.

An additional mode of topical administration is pulmonary, including by oral inhalation and nasal inhalation, which is described in detail below.

Other suitable topical formulations and dosage forms include without limitation ointments, creams, gels, lotions, pastes and the like, as described in Remington: The Science and Practice of Pharmacy, 21st Ed., Lippincott Williams & Wilkins (Philadelphia, Pennsylvania, 2005).

Ointments are semi-solid preparations that are typically based on petrolatum or a petroleum derivative. Creams are viscous liquids or semi-solid emulsions, either oil-in-water or water-in-oil. Cream bases are water-washable, and contain an oil phase, an emulsifier and an aqueous phase. The oil phase, also called the “internal” phase, generally comprises petrolatum and a fatty alcohol (e.g., cetyl or stearyl alcohol). The aqueous phase typically, although not necessarily, exceeds the oil phase in volume, and usually contains a humectant. The emulsifier in a cream formulation is generally a non-ionic, anionic, cationic or amphoteric surfactant. Gels are semi-solid, suspension-type systems. Single-phase gels contain organic macromolecules (polymers) distributed substantially uniformly throughout the carrier liquid, which is typically aqueous but can also contain an alcohol (e.g., ethanol or isopropanol) and optionally an oil. Lotions are preparations to be applied to the skin surface without friction, and are typically liquid or semi-liquid preparations in which solid particles, including the active agent, are present in a water or alcohol base. Lotions are usually suspensions of finely divided solids and typically contain suspending agents to produce better dispersion as well as compounds useful for localizing and holding the active agent in contact with the skin. Pastes are semi-solid dosage forms in which the active agent is suspended in a suitable base. Depending on the nature of the base, pastes are divided between fatty pastes or those made from single-phase aqueous gels.

Various excipients can be included in a topical formulation. For example, solvents, including a suitable amount of an alcohol, can be used to solubilize the active agent. Other optional excipients include without limitation gelling agents, thickening agents, emulsifiers, surfactants, stabilizers, buffers, antioxidants, preservatives, cooling agents (e.g., menthol), opacifiers, fragrances and colorants. For an active agent having a low rate of permeation through the skin or mucosal tissue, a topical formulation can contain a permeation enhancer to increase the permeation of the active agent through the skin or mucosal tissue. A topical formulation can also contain an irritation-mitigating excipient that reduces any irritation to the skin or mucosa caused by the active agent, the permeation enhancer or any other component of the formulation.

In some embodiments, one or more compounds selected from the compounds listed in Tables 2-8 (or a pharmaceutically acceptable salt, ester, solvate, stereoisomer, tautomer, or prodrug thereof) is delivered from a sustained-release composition. As used herein, the term “sustained-release composition” encompasses sustained-release, prolonged-release, extended-release, slow-release and controlled-release compositions, systems and devices. Use of a sustained-release composition can have benefits, such as an improved profile of the amount of the drug or an active metabolite thereof delivered to the target site(s) over a time period, including delivery of a therapeutically effective amount of the drug or an active metabolite thereof over a prolonged time period. In certain embodiments, the sustained-release composition delivers the compound over a period of at least about 1 day, 2 days, 3 days, 1 week, 2 weeks, 3 weeks, 1 month, 2 months, 3 months or longer. In some embodiments, the sustained-release composition is a drug-encapsulation system, such as nanoparticles, microparticles or a capsule made of, e.g., a biodegradable polymer or/and a hydrogel. In certain embodiments, the sustained-release composition comprises a hydrogel. Non-limiting examples of polymers of which a hydrogel can be composed include polyvinyl alcohol, acrylate polymers (e.g., sodium poly acrylate), and other homopolymers and copolymers having a relatively large number of hydrophilic groups (e.g., hydroxyl or/and carboxylate groups). In other embodiments, the sustained-release drug-encapsulation system comprises a membrane-enclosed reservoir, wherein the reservoir contains a drug and the membrane is permeable to the drug. Such a drug-delivery system can be in the form of, e.g., a transdermal patch.

In some embodiments, the sustained-release composition is an oral dosage form, such as a tablet or capsule. For example, a drug can be embedded in an insoluble porous matrix such that the dissolving drag must make its way out of the matrix before it can be absorbed through the gastrointestinal tract. Alternatively, a drug can be embedded in a matrix that swells to form a gel through which the drug exits. Sustained release can also be achieved by way of a single-layer or multi-layer osmotic controlled-release oral delivery system (OROS). An OROS is a tablet with a semi-permeable outer membrane and one or more small laser-drilled holes in it. As the tablet passes through the body, water is absorbed through the semipermeable membrane via osmosis, and the resulting osmotic pressure pushes the drug out through the hole(s) in the tablet and into the gastrointestinal tract where it can be absorbed.

In further embodiments, the sustained-release composition is formulated as polymeric nanoparticles or microparticles, wherein the polymeric particles can be delivered, e.g., by inhalation or injection or from an implant. In some embodiments, the polymeric implant or polymeric nanoparticles or microparticles are composed of a biodegradable polymer. In certain embodiments, the biodegradable polymer comprises lactic acid or/and glycolic acid [e.g., an L-lactic acid-based copolymer, such as poly(L-lactide-co-glycolide) or poly(L-lactic acid-co-D,L-2-hydroxyoctanoic acid)]. For example, biodegradable polymeric microspheres composed of polylactic acid or/and polyglycolic acid can serve as sustained-release pulmonary drug-delivery systems. The biodegradable polymer of the polymeric implant or polymeric nanoparticles or microparticles can be selected so that the polymer substantially completely degrades around the time the period of treatment is expected to end, and so that the byproducts of the polymer's degradation, like the polymer, are biocompatible.

For a delayed or sustained release of one or more compounds selected from the compounds listed in Tables 2-8 (or a pharmaceutically acceptable salt, ester, solvate, stereoisomer, tautomer, or prodrug thereof), a composition can also be formulated as a depot that can be implanted in or injected into a subject, e.g., intramuscularly or subcutaneously. A depot formulation can be designed to deliver the compound over a longer period of time, e.g., over a period of at least about 1 week, 2 weeks, 3 weeks, 1 month, 6 weeks, 2 months, 3 months or longer. For example, the compound can be formulated with a polymeric material (e.g., polyethylene glycol (PEG), polylactic acid (PLA) or polyglycolic acid (PGA), or a copolymer thereof (e.g., PLGA)), a hydrophobic material (e.g., as an emulsion in an oil) or/and an ion-exchange resin, or as a sparingly soluble derivative (e.g., a sparingly soluble salt). As an illustrative example, one or more compounds selected from the compounds listed in Tables 2-8 (or a pharmaceutically acceptable salt, ester, solvate, stereoisomer, tautomer, or prodrug thereof) can be incorporated or embedded in sustained-release microparticles composed of PLGA and formulated as a monthly depot.

One or more compounds selected from the compounds listed in Tables 2-8 (or a pharmaceutically acceptable salt, ester, solvate, stereoisomer, tautomer, or prodrug thereof) can also be contained or dispersed in a matrix material. The matrix material can comprise a polymer (e.g., ethylene-vinyl acetate) and controls the release of the compound by controlling dissolution or/and diffusion of the compound from, e.g., a reservoir, and can enhance the stability of the compound while contained in the reservoir. Such a release system can be designed as a sustained-release system, can be configured as, e.g., a transdermal or transmucosal patch, and can contain an excipient that can accelerate the compound's release, such as a water-swellable material (e.g., a hydrogel) that aids in expelling the compound out of the reservoir. For example, U.S. Pat. Nos. 4,144,317 and 5,797,898 describe examples of such a release system.

The release system can provide a temporally modulated release profile (e.g., pulsatile release) when time variation in plasma levels is desired, or a more continuous or consistent release profile when a constant plasma level is desired. Pulsatile release can be achieved from an individual reservoir or from a plurality of reservoirs. For example, where each reservoir provides a single pulse, multiple pulses (“pulsatile” release) are achieved by temporally staggering the single pulse release from each of multiple reservoirs.

Alternatively, multiple pulses can be achieved from a single reservoir by incorporating several layers of a release system and other materials into a single reservoir. Continuous release can be achieved by incorporating a release system that degrades, dissolves, or allows diffusion of a compound through it over an extended time period. In addition, continuous release can be approximated by releasing several pulses of a compound in rapid succession (“digital” release). An active release system can be used alone or in conjunction with a passive release system, as described in U.S. Pat. No. 5,797,898.

In addition, pharmaceutical compositions comprising one or more compounds selected from the compounds listed in Tables 2-8 (or a pharmaceutically acceptable salt, ester, solvate, stereoisomer, tautomer, or prodrug thereof) can be formulated as, e.g., liposomes, micelles (e.g., those composed of biodegradable natural or/and synthetic polymers, such as lactosomes), microspheres, microparticles or nanoparticles, whether or not designed for sustained release. For example, liposomes can be used as sustained release pulmonary drug-delivery systems that deliver drugs to the alveolar surface for treatment of lung diseases and systemic diseases.

The pharmaceutical compositions can be manufactured in any suitable manner known in the art, e.g., by means of conventional mixing, dissolving, suspending, granulating, dragee-making, levigating, emulsifying, encapsulating, entrapping or compressing processes.

A pharmaceutical composition can be presented in unit dosage form as a single dose wherein all active and inactive ingredients are combined in a suitable system, and components do not need to be mixed to form the composition to be administered. The unit dosage form can contain an effective dose, or an appropriate fraction thereof, of one or more compounds selected from the compounds listed in Tables 2-8 (or a pharmaceutically acceptable salt, ester, solvate, stereoisomer, tautomer, or prodrug thereof). Representative examples of a unit dosage form include a tablet, capsule or pill for oral administration, and powder in a vial or ampoule for oral or nasal inhalation.

Alternatively, a pharmaceutical composition can be presented as a kit, wherein the active ingredient, excipients and carriers (e.g., solvents) are provided in two or more separate containers (e.g., ampoules, vials, tubes, bottles or syringes) and need to be combined to form the composition to be administered. The kit can contain instructions for storing, preparing and administering the composition (e.g., a solution to be injected intravenously).

A kit can contain all active and inactive ingredients in unit dosage form or the active ingredient and inactive ingredients in two or more separate containers, and can contain instructions for using the pharmaceutical composition.

In some embodiments, a kit contains one or more compounds selected from the compounds listed in Tables 2-8 (or a pharmaceutically acceptable salt, ester, solvate, stereoisomer, tautomer, or prodrug thereof) or a pharmaceutically acceptable salt, solvate, hydrate, clathrate, polymorph, prodrug or metabolite thereof, and instructions for administering one or more compounds selected from the compounds listed in Tables 2-8 (or a pharmaceutically acceptable salt, ester, solvate, stereoisomer, tautomer, or prodrug thereof). In certain embodiments, one or more compounds selected from the compounds listed in Tables 2-8 (or a pharmaceutically acceptable salt, ester, solvate, stereoisomer, tautomer, or prodrug thereof) is contained or incorporated in, or provided by, a device or system configured for pulmonary delivery of the compound by oral inhalation, such as a metered-dose inhaler, a dry powder inhaler or a nebulizer.

Inhalation Formulations and Devices

Pulmonary administration can be accomplished by, e.g., oral inhalation or nasal inhalation. Advantages of pulmonary drug delivery include, but are not limited to: 1) avoidance of first pass hepatic metabolism; 2) fast drug action; 3) large surface area of the alveolar region for absorption, high permeability of the lungs (thin air-blood barrier), and profuse vasculature of the airways; 4) smaller doses to achieve equivalent therapeutic effect compared to other oral routes; 5) local action within the respiratory tract; 6) reduced systemic side effects; and 7) reduced extracellular enzyme levels compared to the gastrointestinal tract due to the large alveolar surface area. An advantage of oral inhalation over nasal inhalation includes deeper penetration/deposition of the drug into the lungs. Pulmonary administration, whether by oral or nasal inhalation, can be a suitable route of administration for drugs that are intended to act locally in the lungs or/and systemically, for which the lungs serve as a portal to the systemic circulation.

Oral or nasal inhalation can be achieved by means of, e.g., a metered-dose inhaler (MDI), a nebulizer or a dry powder inhaler (DPI). For example, one or more compounds selected from the compounds listed in Tables 2-8 (or a pharmaceutically acceptable salt, ester, solvate, stereoisomer, tautomer, or prodrug thereof) can be formulated for aerosol administration to the respiratory tract by oral or nasal inhalation. The drug is delivered in a small particle size (e.g., between about 0.5 micron and about 5 microns), which can be obtained by micronization, to improve, e.g., drug deposition in the lungs and drug suspension stability. The drug can be provided in a pressurized pack with a suitable propellant, such as a hydrofluoroalkane (HFA, e.g., 1,1,1,2-tetrafluoroethane [HFA-134a]), a chlorofluorocarbon (CFC, e.g., dichlorodifluoromethane, trichlorofluoromethane or dichlorotetrafluoroethane), or a suitable gas (e.g., oxygen, compressed air or carbon dioxide). The drug in the aerosol formulation is dissolved, or more often suspended, in the propellant for delivery to the lungs. The aerosol can contain excipients such as a surfactant (which enhances penetration into the lungs by reducing the high surface tension forces at the air-water interface within the alveoli, may also emulsify, solubilize or/and stabilize the drug, and can be, e.g., a phospholipid such as lecithin) or/and a stabilizer. For example, an MDI formulation can comprise one or more compounds selected from the compounds listed in Tables 2-8 (or a pharmaceutically acceptable salt, ester, solvate, stereoisomer, tautomer, or prodrug thereof), a propellant (e.g., an HFA such as 1,1,1,2-tetrafluoroethane), a surfactant (e.g., a fatty acid such as oleic acid), and a co-solvent (e.g., an alcohol such as ethanol). The MDI formulation can optionally contain a dissolved gas (e.g., C0₂). After device actuation, the bursting of C0₂ bubbles within the emitted aerosol droplets breaks up the droplets into smaller droplets, thereby increasing the respirable fraction of drug. As another example, a nebulizer formulation can comprise one or more compounds selected from the compounds listed in Tables 2-8 (or a pharmaceutically acceptable salt, ester, solvate, stereoisomer, tautomer, or prodrug thereof), a surfactant (e.g., a Tween® such as polysorbate 80), a chelator or preservative (e.g., edetate disodium), an isotonicity agent (e.g., sodium chloride), pH buffering agents (e.g., citric acid/sodium citrate), and water. The drug can be delivered by means of, e.g., a nebulizer or an MDI with or without a spacer, and the drug dose delivered can be controlled by a metering chamber (nebulizer) or a metering valve (MDI).

Metered-dose inhalers (also called pressurized metered-dose inhalers [pMDI]) are the most widely used inhalation devices. A metering valve delivers a precise amount of aerosol (e.g., about 20-100 μL) each time the device is actuated. MDIs typically generate aerosol faster than the user can inhale, which can result in deposition of much of the aerosol in the mouth and the throat. The problem of poor coordination between device actuation and inhalation can be addressed by using, e.g., a breath-actuated MDI or a coordination device. A breath-actuated MDI (e.g., Easibreathe®) is activated when the device senses the user's inspiration and discharges a drug dose in response. The inhalation flow rate is coordinated through the actuator and the user has time to actuate the device reliably during inhalation. In a coordination device, a spacer (or valved holding chamber), which is a tube attached to the mouthpiece end of the inhaler, serves as a reservoir or chamber holding the drug that is sprayed by the inhaler and reduces the speed at which the aerosol enters the mouth, thereby allowing for the evaporation of the propellant from larger droplets. The spacer simplifies use of the inhaler and increases the amount of drug deposited in the lungs instead of in the upper airways. The spacer can be made of an anti-static polymer to minimize electrostatic adherence of the emitted drug particles to the inner walls of the spacer.

Nebulizers generate aerosol droplets of about 1-5 microns. They do not require user coordination between device actuation and inhalation, which can significantly affect the amount of drug deposited in the lungs. Compared to MDIs and DPIs, nebulizers can deliver larger doses of drug, albeit over a longer administration time. Examples of nebulizers include without limitation human-powered nebulizers, jet nebulizers (e.g., AeroEclipse® II BAN [breath-actuated], CompAIR™ NE-C801 [virtual valve], PARI LC® Plus [breath-enhanced] and SideStream Plus [breath-enhanced]), ultrasonic wave nebulizers, and vibrating mesh nebulizers (e.g., Akita2® Apixneb, I-neb AAD System with metering chambers, Micro Air® NE-U22, Omron U22 and PARI eFlow® rapid). As an example, a pulsed ultrasonic nebulizer can aerosolize a fixed amount of the drug per pulse, and can comprise an opto-acoustical trigger that allows the user to synchronize each breath to each pulse.

Respimat® Soft Mist™ inhaler combines advantages of an MDI and a nebulizer. It is a small, hand-held inhaler that does not need a power supply (like an MDI) and slowly aerosolizes a propellant-free drug solution as a soft mist (like a nebulizer), thereby reducing drug deposition in the oropharyngeal region and increasing drug deposition in the central and peripheral lung regions. The Soft Mist™ inhaler can create a large fraction of respirable droplets with slow velocity from a metered volume of drug solution. A drug delivered from the Soft Mist™ inhaler can potentially achieve the same therapeutic outcome at a significantly lower dose compared to delivery from an MDI.

For oral or nasal inhalation using a dry powder inhaler (DPI), one or more compounds selected from the compounds listed in Tables 2-8 (or a pharmaceutically acceptable salt, ester, solvate, stereoisomer, tautomer, or prodrug thereof) can be provided in the form of a dry micronized powder, where the drug particles are of a certain small size (e.g., between about 0.5 micron and about 5 microns) to improve, e.g., aerodynamic properties of the dispersed powder and drug deposition in the lungs. Particles between about 0.5 micron and about 5 microns deposit by sedimentation in the terminal bronchioles and the alveolar regions. By contrast, the majority of larger particles (>5 microns) do not follow the stream of air into the many bifurcations of the airways, but rather deposit by impaction in the upper airways, including the oropharyngeal region of the throat. A DPI formulation can contain the drug particles alone or blended with a powder of a suitable larger base/carrier, such as lactose, starch, a starch derivative (e.g., hydroxypropylmethyl cellulose) or polyvinylpyrrolidine. The carrier particles enhance flow, reduce aggregation, improve dose uniformity and aid in dispersion of the drug particles. A DPI formulation can optionally contain an excipient such as magnesium stearate or/and leucine that improves the performance of the formulation by interfering with inter-particle bonding (by anti-adherent action). The powder formulation can be provided in unit dose form, such as a capsule (e.g., a gelatin capsule) or a cartridge in a blister pack, which can be manually loaded or pre-loaded in an inhaler. The drug particles can be drawn into the lungs by placing the mouthpiece or nosepiece of the inhaler into the mouth or nose, taking a sharp, deep inhalation to create turbulent airflow, and holding the breath for a period of time (e.g., about 5-10 seconds) to allow the drug particles to settle down in the bronchioles and the alveolar regions. When the user actuates the DPI and inhales, airflow through the device creates shear and turbulence, inspired air is introduced into the powder bed, and the static powder blend is fluidized and enters the user's airways. There, the drug particles separate from the carrier particles due to turbulence and are carried deep into the lungs, while the larger carrier particles impact on the oropharyngeal surfaces and are cleared. Thus, the user's inspiratory airflow achieves powder de-agglomeration and aeroionisation, and determines drug deposition in the lungs. (While a passive DPI requires rapid inspiratory airflow to de-agglomerate drug particles, rapid inspiration is not recommended with an MDI or nebulizer, since it creates turbulent airflow and fast velocity which increase drug deposition by impaction in the upper airways.) Compared to an MDI, a DPI (including a passive, breath-activated DPI) can potentially deliver larger doses of drug, and larger-size drugs (e.g., macromolecules), to the lungs.

Lactose (e.g., alpha-lactose monohydrate) is the most commonly used carrier in DPI formulations. Examples of grades/types of lactose monohydrate for DPI formulations include without limitation DCL 11, Flowlac® 100, Inhalac® 230, Lactohale® 300, Lactopress® SD 250 (spray-dried lactose), Respitose® SV003 and Sorbolac® 400. A DPI formulation can contain a single lactose grade or a combination of different lactose grades. For example, a fine lactose grade like Lactohale® 300 or Sorbolac® 400 may not be a suitable DPI carrier and may need to be blended with a coarse lactose grade like DCL 11, Flowlac® 100, Inhalac® 230 or Respitose® SV003 (e.g., about a 1:9 ratio of fine lactose to coarse lactose) to improve flow. The distribution of the carrier particle sizes affects the fine particle fraction/dose (FPF or FPD) of the drug, with a high FPF being desired for drug delivery to the lungs. FPF/FPD is the respirable fraction/dose mass out of the DPI device with an aerodynamic particle size<5 microns in the inspiration air. High FPF, and hence good DPI performance, can be obtained from, e.g., DPI formulations having an approximately 1:9 ratio of fine lactose (e.g., Lactohale® 300) to coarse lactose (e.g., Respitose® SV003) and about 20% w/w overages to avoid deposition of the drug in the capsule shell or the DPI device and to deliver essentially all of the drug to the airways.

Other carriers for DPI formulations include without limitation glucose, mannitol (e.g., crystallized mannitol [Pearlitol 110 C] and spray-dried mannitol [Pearlitol 100 SD]), maltitol (e.g., crystallized maltitol [Maltisorb P90]), sorbitol and xylitol.

To improve the performance of DPI formulations, pulmospheres can be used. These relatively large porous, hollow particles have low particle density and improved dispersibility. Pulmospheres can be prepared using a polymeric or non-polymeric excipient by, e.g., solvent evaporation or spray drying. For example, pulmospheres can be made of phosphatidylcholine, the primary component of human lung surfactant. The relatively large size of pulmospheres allows them to remain in the alveolar region longer than their non-porous counterparts by avoiding phagocytic clearance. Pulmospheres can also be used in aerosol formulations for MDIs as well as for DPIs.

Dry powder inhalers can be classified by dose type into single-unit dose (including disposable and reusable) and multi-dose (including multi-dose reservoirs and multi-unit dose). In a single-unit dose DPI, the formulation can be a powder mix of a micronized drug powder and a carrier and can be supplied in individual capsules, which are inserted into the inhaler for a single dose and are removed and discarded after use. The capsule body containing the dose falls into the device, while the cap is retained in the entry port for subsequent disposal. As the user inhales, the portion of the capsule containing the drug experiences erratic motion in the airstream, causing dislodged particles to be entrained and subsequently inhaled. Particle de-aggregation is caused mainly by turbulence promoted by the grid upstream of the mouthpiece or nosepiece. Examples of single-unit dose DPIs include without limitation Aerolizer®, AIR®, Conix One® (foil seal), Diskhaler®, Diskus®, Handihaler®, Microhaler®, Rotahaler® and Turbo Spin®.

A multi-unit dose DPI uses factory-metered and -sealed doses packaged in a manner so that the device can hold multiple doses without the user having to reload. The packaging typically contains replaceable disks or cartridges, or strips of foil-polymer blister packaging that may or may not be reloadable. For example, individual doses can be packaged in blister packs on a disk cassette. Following piercing, inspiratory flow through the packaging depression containing the drug induces dispersion of the powder. The aerosol stream is mixed with a bypass flow entering through holes in the mouthpiece or nosepiece, which gives rise to turbulence and promotes particle de-agglomeration. Advantages of the prepackaging include protection from the environment until use and ensurance of adequate control of dose uniformity. Examples of multi-unit dose DPIs include without limitation Acu-Breath®, Bulkhaler®, Certihaler®, DirectHaler®, Diskhaler®, Diskus®, Dispohaler®, M®, MF-DPI®, Miat-Haler®, NEXT DPI®, Prohaler®, Swinhaler® and Technohaler®.

A multi-dose reservoir DPI stores the formulation in bulk, and has a built-in mechanism to meter individual doses from the bulk upon actuation. It contains multiple doses of small pellets of micronized drug that disintegrate into their primary particles during metering and inhalation. One dose can be dispensed into the dosing chamber by a simple back-and-forth twisting action on the base of the reservoir. Scrapers actively force the drug into conical holes, which causes the pellets to disintegrate. Fluidization of the powder is achieved by shear force as air enters the inhaler, and particle de-agglomeration occurs via turbulence. Advantages of multi-dose reservoir DPIs include their relative ease and low cost of manufacture, and the ease of inclusion of a large number of doses within the device. Examples of multi-dose reservoir DPIs include without limitation Acu-Breath®, Airmax®, Bulkhaler®, Certihaler®, Clickhaler®, Cyclovent®, Dispohaler®, JAGO®, MF-DPI®, Miat-Haler®, NEXT DPI®, Swinhaler® and Turbuhaler®.

Most DPIs are breath-activated (“passive”), relying on the user's inhalation for aerosol generation. Examples of passive DPIs include without limitation Airmax®, Novolizer®, Otsuka DPI (compact cake), and the DPIs mentioned above. The air classifier technology (ACT) is an efficient passive powder dispersion mechanism employed in DPIs. In ACT, multiple supply channels generate a tangential airflow that results in a cyclone within the device during inhalation. There are also power-assisted (“active”) DPIs (based on, e.g., pneumatics, impact force or vibration) that use energy to aid, e.g., particle de-agglomeration. For example, the active mechanism of Exubera® inhalers utilizes mechanical energy stored in springs or compressed-air chambers. Examples of active DPIs include without limitation Actispire® (single-unit dose), Aspirair® (multi-dose), Exubera® (single-unit dose), MicroDose® (multi-unit dose and electronically activated), Omnihaler® (single-unit dose), Pfeiffer DPI (single-unit dose), and Spiros® (multi-unit dose).

### RNA Viruses

Disclosed herein include methods for preventing, delaying the onset of, or treating an infection, disease, or inflammation caused by a RNA virus. The present disclosure contemplates treating a broad range of viral diseases, including infections of all types, locations, sizes, and characteristics. The RNA virus can be a double-stranded RNA virus. The RNA virus can be a positive-sense single-stranded RNA virus. The positive-sense single-stranded RNA virus can be a coronavirus. The coronavirus can be an alpha coronavirus, a beta coronavirus, a gamma coronavirus, or a delta coronavirus. The coronavirus can be Middle East respiratory coronavirus (MERS-CoV), severe acute respiratory syndrome coronavirus (SARS-CoV), or SARS-CoV-2.

The infection or disease caused by the RNA virus can be common cold, influenza, SARS, coronaviruses, COVID-19, hepatitis C, hepatitis E, West Nile fever, Ebola virus disease, rabies, polio, or measles.

The methods and compositions disclosed herein are useful for preventing, delaying the onset of, or treating an infection, disease, or inflammation caused by a RNA virus. The subject can have been exposed to the RNA virus, can be suspected to have been exposed to the RNA virus, or can be at a risk of being exposed to the RNA virus. The compositions may be used as a prophylactic (to prevent the development of a viral infection) or may be used to treat existing viral infections.

The RNA virus can be an enveloped virus. The RNA virus can a retrovirus. The RNA virus can be a filovirus, arenavirus, bunyavirus, or a rhabdovirus. The RNA virus can be a hepadnavirus, coronavirus, or a flavivirus. The RNA virus can be Respiratory syncytial virus, Parainfluenza virus, Enterovirus 71, Hantavirus, SARS virus, SARS-associated coronavirus, severe acute respiratory syndrome coronavirus (SARS-CoV), or SARS-CoV-2, Sin Nombre virus, Respiratory reovirus. The present disclosure encompasses the treatment of infections with derivatives of any of the viruses disclosed herein. As disclosed herein, the term “derivative of a virus” can refer to a strain of virus that has mutated from an existing viral strain.

The RNA virus can comprise any serotype of human rhinovirus (HRV). HRV may include, without limitation, the species Rhinovirus A (including, but not limited to, serotypes HRV-A1, HRV-A2, HRV-A7, HRV-A8, HRV-A9, HRV-A10, HRV-A11, HRV-A12, HRV-A13, HRV-A15, HRV-A16, HRV-A18, HRV-A19, HRV-A20, HRV-A21, HRV-A22, HRV-A23, HRV-A24, HRV-A25, HRV-A28, HRV-A29, HRV-A30, HRV-A31, HRV-A32, HRV-A33, HRV-A34, HRV-A36, HRV-A38, HRV-A39, HRV-A40, HRV-A41, HRV-A43, HRV-A44, HRV-A45, HRV-A46, HRV-A47, HRV-A49, HRV-A50, HRV-A51, HRV-A53, HRV-A54, HRV-A55, HRV-A56, HRV-A57, HRV-A58, HRV-A59, HRV-A60, HRV-A61, HRV-A62, HRV-A63, HRV-A64, HRV-A65, HRV-A66, HRV-A67, HRV-A68, HRV-A71, HRV-A73, HRV-A74, HRV-A75, HRV-A76, HRV-A77, HRV-A78, HRV-A80, HRV-A81, HRV-A82, HRV-A85, HRV-A88, HRV-A89, HRV-A90, HRV-A94, HRV-A95, HRV-A96, HRV-A98, HRV-A100, HRV-A101, HRV-A102 and HRV-A103), Rhino virus B (including, but not limited to, the serotypes HRV-B3, HRV-B4, HRV-B5, HRV-B6, HRV-B14, HRV-B17, HRV-B26, HRV-B27, HRV-B35, HRV-B37, HRV-B42, HRV-B48, HRV-B52, HRV-B69, HRV-B70, HRV-B72, HRV-B79, HRV-B83, HRV-B84, HRV-B86, HRV-B91, HRV-B92, HRV-B93, HRV-B97, and HRV-B99), and Rhinovirus C (including, but not limited to, serotypes HRV-C1, HRV-C2, HRV-C3, HRV-C4, HRV-C5, HRV-C6, HRV-C7, HRV-C8, HRV-C9, HRV-C10, HRV-C11, HRV-C12, HRV-C13, HRV-C14, HRV-C15, HRV-C16, HRV-C17, HRV-C18, HRV-C19, HRV-C20, HRV-C21, HRV-C22, HRV-C23, HRV-C24, HRV-C25, HRV-C26, HRV-C27, HRV-C28, HRV-C29, HRV-C30, HRV-C31, HRV-C32, HRV-C33, HRV-C34, HRV-C35, HRV-C36, HRV-C37, HRV-C38, HRV-C39, HRV-C40, HRV-C41, HRV-C42, HRV-C43, HRV-C44, HRV-C45, HRV-C46, HRV-C47, HRV-C48, HRV-C49, HRV-C50 and HRV-C51).

In some embodiments the RNA virus is an influenza A virus. Non-limiting examples of influenza A viruses include subtype H10N4, subtype H10N5, subtype H10N7, subtype H10N8, subtype H10N9, subtype H11N1, subtype H11N13, subtype H11N2, subtype H11N4, subtype H11N6, subtype H11N8, subtype H11N9, subtype H12N1, subtype H12N4, subtype H12N5, subtype H12N8, subtype H13N2, subtype H13N3, subtype H13N6, subtype H13N7, subtype H14N5, subtype H14N6, subtype H15N8, subtype H15N9, subtype H16N3, subtype H1N1, subtype H1N2, subtype H1N3, subtype H1N6, subtype H1N9, subtype H2N1, subtype H2N2, subtype H2N3, subtype H2N5, subtype H2N7, subtype H2N8, subtype H2N9, subtype H3N1, subtype H3N2, subtype H3N3, subtype H3N4, subtype H3N5, subtype H3N6, subtype H3N8, subtype H3N9, subtype H4N1, subtype H4N2, subtype H4N3, subtype H4N4, subtype H4N5, subtype H4N6, subtype H4N8, subtype H4N9, subtype H5N1, subtype H5N2, subtype H5N3, subtype H5N4, subtype H5N6, subtype H5N7, subtype H5N8, subtype H5N9, subtype H6N1, subtype H6N2, subtype H6N3, subtype H6N4, subtype H6N5, subtype H6N6, subtype H6N7, subtype H6N8, subtype H6N9, subtype H7N1, subtype H7N2, subtype H7N3, subtype H7N4, subtype H7N5, subtype H7N7, subtype H7N8, subtype H7N9, subtype H8N4, subtype H8N5, subtype H9N1, subtype H9N2, subtype H9N3, subtype H9N5, subtype H9N6, subtype H9N7, subtype H9N8, and subtype H9N9.

Specific examples of strains of influenza A virus include, but are not limited to: A/sw/Iowa/15/30 (H1N1); A/WSN/33 (H1N1); A/eq/Prague/1/56 (H7N7); A/PR/8/34; A/mallard/Potsdam/178-4/83 (H2N2); A/herring gull/DE/712/88 (H16N3); A/sw/Hong Kong/168/1993 (H1N1); A/mallard/Alberta/211/98 (H1N1); A/shorebird/Delaware/168/06 (H16N3); A/sw/Netherlands/25/80 (H1N1); A/sw/Germany/2/81 (H1N1); A/sw/Hannover/1/81 (H1N1); A/sw/Potsdam/1/81 (H1N1); A/sw/Potsdam/15/81 (H1N1); A/sw/Potsdam/268/81 (H1N1); A/sw/Finistere/2899/82 (H1N1); A/sw/Potsdam/35/82 (H3N2); A/sw/Cote d'Armor/3633/84 (H3N2); A/sw/Gent/1/84 (H3N2); A/sw/Netherlands/12/85 (H1N1); A/sw/Karrenzien/2/87 (H3N2); A/sw/Schwerin/103/89 (H1N1); A/turkey/Germany/3/91 (H1N1); A/sw/Germany/8533/91 (H1N1); A/sw/Belgium/220/92 (H3N2); A/sw/GentN230/92 (H1N1); A/sw/Leipzig/145/92 (H3N2); A/sw/Re220/92 hp (H3N2); A/sw/Bakum/909/93 (H3N2); A/sw/Schleswig-Holstein/1/93 (H1N1); A/sw/Scotland/419440/94 (H1N2); A/sw/Bakum/5/95 (H1N1); A/sw/Best/5C/96 (H1N1); A/sw/England/17394/96 (H1N2); A/sw/Jena/5/96 (H3N2); A/sw/Oedenrode/7C/96 (H3N2); A/sw/Lohne/1/97 (H3N2); A/sw/Cote d'Armor/790/97 (H1N2); A/sw/Bakum/1362/98 (H3N2); A/sw/Italy/1521/98 (H1N2); A/sw/Italy/1553-2/98 (H3N2); A/sw/Italy/1566/98 (H1N1); A/sw/Italy/1589/98 (H1N1); A/sw/Bakum/8602/99 (H3N2); A/sw/Cotes d'Armor/604/99 (H1N2); A/sw/Cote d'Armor/1482/99 (H1N1); A/sw/Gent/7625/99 (H1N2); A/Hong Kong/1774/99 (H3N2); A/sw/Hong Kong/5190/99 (H3N2); A/sw/Hong Kong/5200/99 (H3N2); A/sw/Hong Kong/5212/99 (H3N2); A/sw/Ille et Villaine/1455/99 (H1N1); A/sw/Italy/1654-1/99 (H1N2); A/sw/Italy/2034/99 (H1N1); A/sw/Italy/2064/99 (H1N2); A/sw/Berlin/1578/00 (H3N2); A/sw/Bakum/1832/00 (H1N2); A/sw/Bakum/1833/00 (H1N2); A/sw/Cote d'Armor/800/00 (H1N2); A/sw/Hong Kong/7982/00 (H3N2); A/sw/Italy/1081/00 (H1N2); A/sw/Belzig/2/01 (H1N1); A/sw/Belzig/54/01 (H3N2); A/sw/Hong Kong/9296/01 (H3N2); A/sw/Hong Kong/9745/01 (H3N2); A/sw/Spain/33601/01 (H3N2); A/sw/Hong Kong/1144/02 (H3N2); A/sw/Hong Kong/1197/02 (H3N2); A/sw/Spain/39139/02 (H3N2); A/sw/Spain/42386/02 (H3N2); A/Switzerland/8808/2002 (H1N1); A/sw/Bakum/1769/03 (H3N2); A/sw/Bissendorf/IDT1864/03 (H3N2); A/sw/Ehren/IDT2570/03 (H1N2); A/sw/Gescher/IDT2702/03 (H1N2); A/sw/Haselünne/2617/03 hp (H1N1); A/sw/Loningen/IDT2530/03 (H1N2); A/sw/IVD/IDT2674/03 (H1N2); A/sw/Nordkirchen/IDT1993/03 (H3N2); A/sw/Nordwalde/IDT2197/03 (H1N2); A/sw/Norden/IDT2308/03 (H1N2); A/sw/Spain/50047/03 (H1N1); A/sw/Spain/51915/03 (H1N1); A/sw/Vechta/2623/03 (H1N1); A/sw/Visbek/IDT2869/03 (H1N2); A/sw/Waltersdorf/IDT2527/03 (H1N2); A/sw/Damme/IDT2890/04 (H3N2); A/sw/Geldern/IDT2888/04 (H1N1); A/sw/Granstedt/IDT3475/04 (H1N2); A/sw/Greven/IDT2889/04 (H1N1); A/sw/Gudensberg/IDT2930/04 (H1N2); A/sw/Gudensberg/IDT2931/04 (H1N2); A/sw/Lohne/IDT3357/04 (H3N2); A/sw/Nortrup/IDT3685/04 (H1N2); A/sw/Seesen/IDT3055/04 (H3N2); A/sw/Spain/53207/04 (H1N1); A/sw/Spain/54008/04 (H3N2); A/sw/Stolzenau/IDT3296/04 (H1N2); A/sw/Wedel/IDT2965/04 (H1N1); A/sw/Bad Griesbach/IDT4191/05 (H3N2); A/sw/Cloppenburg/IDT4777/05 (H1N2); A/sw/Dotlingen/IDT3780/05 (H1N2); A/sw/Dotlingen/IDT4735/05 (H1N2); A/sw/Egglham/IDT5250/05 (H3N2); A/sw/Harkenblek/IDT4097/05 (H3N2); A/sw/Hertzen/IDT4317/05 (H3N2); A/sw/Krogel/IDT4192/05 (H1N1); A/sw/Laer/IDT3893/05 (H1N1); A/sw/Laer/IDT4126/05 (H3N2); A/sw/Merzen/IDT4114/05 (H3N2); A/sw/Muesleringen-S./IDT4263/05 (H3N2); A/sw/Osterhofen/IDT4004/05 (H3N2); A/sw/Sprenge/IDT3805/05 (H1N2); A/sw/Stadtlohn/IDT3853/05 (H1N2); A/swNoglarn/IDT4096/05 (H1N1); A/sw/Wohlerst/IDT4093/05 (H1N1); A/sw/Bad Griesbach/IDT5604/06 (H1N1); A/sw/Herzlake/IDT5335/06 (H3N2); A/sw/Herzlake/IDT5336/06 (H3N2); A/sw/Herzlake/IDT5337/06 (H3N2); and A/wild boar/Germany/R169/2006 (H3N2).

Other specific examples of strains of influenza A virus include, but are not limited to: A/Toronto/3141/2009 (H1N1); A/Regensburg/D6/2009 (H1N1); A/Bayern/62/2009 (H1N1); A/Bayern/62/2009 (H1N1); A/Bradenburg/19/2009 (H1N1); A/Bradenburg/20/2009 (H1N1); A/Distrito Federal/2611/2009 (H1N1); A/Mato Grosso/2329/2009 (H1N1); A/Sao Paulo/1454/2009 (H1N1); A/Sao Paulo/2233/2009 (H1N1); A/Stockholm/37/2009 (H1N1); A/Stockholm/41/2009 (H1N1); A/Stockholm/45/2009 (H1N1); A/swine/Alberta/OTH-33-1/2009 (H1N1); A/swine/Alberta/OTH-33-14/2009 (H1N1); A/swine/Alberta/OTH-33-2/2009 (H1N1); A/swine/Alberta/OTH-33-21/2009 (H1N1); A/swine/Alberta/OTH-33-22/2009 (H1N1); A/swine/Alberta/OTH-33-23/2009 (H1N1); A/swine/Alberta/OTH-33-24/2009 (H1N1); A/swine/Alberta/OTH-33-25/2009 (H1N1); A/swine/Alberta/OTH-33-3/2009 (H1N1); A/swine/Alberta/OTH-33-7/2009 (H1N1); A/Beijing/502/2009 (H1N1); A/Firenze/10/2009 (H1N1); A/Hong Kong/2369/2009 (H1N1); A/Italy/85/2009 (H1N1); A/Santo Domingo/572N/2009 (H1N1); A/Catalonia/385/2009 (H1N1); A/Catalonia/386/2009 (H1N1); A/Catalonia/387/2009 (H1N1); A/Catalonia/390/2009 (H1N1); A/Catalonia/394/2009 (H1N1); A/Catalonia/397/2009 (H1N1); A/Catalonia/398/2009 (H1N1); A/Catalonia/399/2009 (H1N1); A/Sao Paulo/2303/2009 (H1N1); A/Akita/1/2009 (H1N1); A/Castro/JXP/2009 (H1N1); A/Fukushima/1/2009 (H1N1); A/Israel/276/2009 (H1N1); A/Israel/277/2009 (H1N1); A/Israel/70/2009 (H1N1); A/Iwate/1/2009 (H1N1); A/Iwate/2/2009 (H1N1); A/Kagoshima/1/2009 (H1N1); A/Osaka/180/2009 (H1N1); A/Puerto Montt/Bio87/2009 (H1N1); A/Sao Paulo/2303/2009 (H1N1); A/Sapporo/1/2009 (H1N1); A/Stockholm/30/2009 (H1N1); A/Stockholm/31/2009 (H1N1); A/Stockholm/32/2009 (H1N1); A/Stockholm/33/2009 (H1N1); A/Stockholm/34/2009 (H1N1); A/Stockholm/35/2009 (H1N1); A/Stockholm/36/2009 (H1N1); A/Stockholm/38/2009 (H1N1); A/Stockholm/39/2009 (H1N1); A/Stockholm/40/2009 (H1N1;) A/Stockholm/42/2009 (H1N1); A/Stockholm/43/2009 (H1N1); A/Stockholm/44/2009 (H1N1); A/Utsunomiya/2/2009 (H1N1); A/WRAIR/0573N/2009 (H1N1); and A/Zhejiang/DTID-ZJU01/2009 (H1N1).

In some embodiments the RNA virus is an influenza B virus. Non-limiting examples of influenza B viruses include strain Aichi/5/88, strain Akita/27/2001, strain Akita/5/2001, strain Alaska/16/2000, strain Alaska/1777/2005, strain Argentina/69/2001, strain Arizona/146/2005, strain Arizona/148/2005, strain Bangkok/163/90, strain Bangkok/34/99, strain Bangkok/460/03, strain Bangkok/54/99, strain Barcelona/215/03, strain Beijing/15/84, strain Beijing/184/93, strain Beijing/243/97, strain Beijing/43/75, strain Beijing/5/76, strain Beijing/76/98, strain Belgium/WV106/2002, strain Belgium/WV107/2002, strain Belgium/WV109/2002, strain Belgium/WV114/2002, strain Belgium/WV122/2002, strain Bonn/43, strain Brazil/952/2001, strain Bucharest/795/03, strain Buenos Aires/161/00), strain Buenos Aires/9/95, strain Buenos Aires/SW16/97, strain Buenos AiresNL518/99, strain Canada/464/2001, strain Canada/464/2002, strain Chaco/366/00, strain Chaco/R113/00, strain Cheju/303/03, strain Chiba/447/98, strain Chongqing/3/2000, strain clinical isolate SA1 Thailand/2002, strain clinical isolate SA10 Thailand/2002, strain clinical isolate SA100 Philippines/2002, strain clinical isolate SA101 Philippines/2002, strain clinical isolate SA1 10 Philippines/2002), strain clinical isolate SA112 Philippines/2002, strain clinical isolate SA113 Philippines/2002, strain clinical isolate SA114 Philippines/2002, strain clinical isolate SA2 Thailand/2002, strain clinical isolate SA20 Thailand/2002, strain clinical isolate SA38 Philippines/2002, strain clinical isolate SA39 Thailand/2002, strain clinical isolate SA99 Philippines/2002, strain CNIC/27/2001, strain Colorado/2597/2004, strain CordobaNA418/99, strain Czechoslovakia/16/89, strain Czechoslovakia/69/90, strain Daeku/10/97, strain Daeku/45/97, strain Daeku/47/97, strain Daeku/9/97, strain B/Du/4/78, strain B/Durban/39/98, strain Durban/43/98, strain Durban/44/98, strain B/Durban/52/98, strain Durban/55/98, strain Durban/56/98, strain England/1716/2005, strain England/2054/2005), strain England/23/04, strain Finland/154/2002, strain Finland/159/2002, strain Finland/160/2002, strain Finland/161/2002, strain Finland/162/03, strain Finland/162/2002, strain Finland/162/91, strain Finland/164/2003, strain Finland/172/91, strain Finland/173/2003, strain Finland/176/2003, strain Finland/184/91, strain Finland/188/2003, strain Finland/190/2003, strain Finland/220/2003, strain Finland/WV5/2002, strain Fujian/36/82, strain Geneva/5079/03, strain Genoa/11/02, strain Genoa/2/02, strain Genoa/21/02, strain Genova/54/02, strain Genova/55/02, strain Guangdong/05/94, strain Guangdong/08/93, strain Guangdong/5/94, strain Guangdong/55/89, strain Guangdong/8/93, strain Guangzhou/7/97, strain Guangzhou/86/92, strain Guangzhou/87/92, strain Gyeonggi/592/2005, strain Hannover/2/90, strain Harbin/07/94, strain Hawaii/10/2001, strain Hawaii/1990/2004, strain Hawaii/38/2001, strain Hawaii/9/2001, strain Hebei/19/94, strain Hebei/3/94), strain Henan/22/97, strain Hiroshima/23/2001, strain Hong Kong/110/99, strain Hong Kong/1115/2002, strain Hong Kong/112/2001, strain Hong Kong/123/2001, strain Hong Kong/1351/2002, strain Hong Kong/1434/2002, strain Hong Kong/147/99, strain Hong Kong/156/99, strain Hong Kong/157/99, strain Hong Kong/22/2001, strain Hong Kong/22/89, strain Hong Kong/336/2001, strain Hong Kong/666/2001, strain Hong Kong/9/89, strain Houston/1/91, strain Houston/1/96, strain Houston/2/96, strain Hunan/4/72, strain Ibaraki/2/85, strain ncheon/297/2005, strain India/3/89, strain India/77276/2001, strain Israel/95/03, strain Israel/WV187/2002, strain Japan/1224/2005, strain Jiangsu/10/03, strain Johannesburg/1/99, strain Johannesburg/96/01, strain Kadoma/1076/99, strain Kadoma/122/99, strain Kagoshima/15/94, strain Kansas/22992/99, strain Khazkov/224/91, strain Kobe/1/2002, strain, strain Kouchi/193/99, strain Lazio/1/02, strain Lee/40, strain Leningrad/129/91, strain Lissabon/2/90), strain Los Angeles/1/02, strain Lusaka/270/99, strain Lyon/1271/96, strain Malaysia/83077/2001, strain Maputo/1/99, strain Mar del Plata/595/99, strain Maryland/i/01, strain Memphis/i/01, strain Memphis/12/97-MA, strain Michigan/22572/99, strain Mie/1/93, strain Milano/1/01, strain Minsk/318/90, strain Moscow/3/03, strain Nagoya/20/99, strain Nanchang/1/00, strain Nashville/107/93, strain Nashville/45/91, strain Nebraska/2/01, strain Netherland/801/90, strain Netherlands/429/98, strain New York/1/2002, strain NIB/48/90, strain Ningxia/45/83, strain Norway/1/84, strain Oman/16299/2001, strain Osaka/1059/97, strain Osaka/983/97-V2, strain Oslo/1329/2002, strain Oslo/1846/2002, strain Panama/45/90, strain Paris/329/90, strain Parma/23/02, strain Perth/211/2001, strain Peru/1364/2004, strain Philippines/5072/2001, strain Pusan/270/99, strain Quebec/173/98, strain Quebec/465/98, strain Quebec/7/01, strain Roma/1/03, strain Saga/S172/99, strain Seoul/13/95, strain Seoul/37/91, strain Shangdong/7/97, strain Shanghai/361/2002), strain Shiga/T30/98, strain Sichuan/379/99, strain Singapore/222/79, strain Spain/WV27/2002, strain Stockholm/10/90, strain Switzerland/5441/90, strain Taiwan/0409/00, strain Taiwan/0722/02, strain Taiwan/97271/2001, strain Tehran/80/02, strain Tokyo/6/98, strain Trieste/28/02, strain Ulan Ude/4/02, strain United Kingdom/34304/99, strain USSR/100/83, strain Victoria/103/89, strain Vienna/1/99, strain Wuhan/356/2000, strain WV194/2002, strain Xuanwu/23/82, strain Yamagata/1311/2003, strain Yamagata/K500/2001, strain Alaska/12/96, strain GA/86, strain NAGASAKI/1/87, strain Tokyo/942/96, and strain Rochester/02/2001.

The RNA virus can be an influenza C virus, for example strain Aichi/1/81, strain Ann Arbor/i/50, strain Aomori/74, strain California/78, strain England/83, strain Greece/79, strain Hiroshima/246/2000, strain Hiroshima/252/2000, strain Hyogo/1/83, strain Johannesburg/66, strain Kanagawa/1/76, strain Kyoto/1/79, strain Mississippi/80, strain Miyagi/1/97, strain Miyagi/5/2000, strain Miyagi/9/96, strain Nara/2/85, strain NewJersey/76, strain pig/Beijing/115/81, strain Saitama/3/2000), strain Shizuoka/79, strain Yamagata/2/98, strain Yamagata/6/2000, strain Yamagata/9/96, strain BERLIN/1/85, strain ENGLAND/892/8, strain GREAT LAKES/1167/54, strain JJ/50, strain PIG/BEIJING/10/81, strain PIG/BEIJING/439/82), strain TAYLOR/1233/47, and strain C/YAMAGATA/10/81.

### Combination Therapies and Additional Therapeutic Agents

Disclosed herein include methods for preventing, delaying the onset of, or treating an infection or a disease caused by a RNA virus. In some embodiments, the method comprises: administering to a subject in need thereof (1) a first compound selected from the compounds listed in Tables 2-8, or a pharmaceutically acceptable salt, ester, solvate, stereoisomer, tautomer, or prodrug thereof, and (2) a second compound selected from the compounds listed in Tables 2-8, or a pharmaceutically acceptable salt, ester, solvate, stereoisomer, tautomer, or prodrug thereof, thereby preventing, delaying the onset of, or treating the inflammatory effect, wherein the first compound and the second compound are different.

Disclosed herein include methods for preventing, delaying the onset of, or treating an inflammatory effect of an infection or a disease caused by a RNA virus. In some embodiments, the method comprises: administering to a subject in need thereof (1) a first compound selected from the compounds listed in Tables 2-8, or a pharmaceutically acceptable salt, ester, solvate, stereoisomer, tautomer, or prodrug thereof, and (2) a second compound selected from the compounds listed in Tables 2-8, or a pharmaceutically acceptable salt, ester, solvate, stereoisomer, tautomer, or prodrug thereof, thereby preventing, delaying the onset of, or treating the inflammatory effect, wherein the first compound and the second compound are different.

The method can comprise: administering to the subject (3) a third compound selected from the compounds listed in Tables 2-8, or a pharmaceutically acceptable salt, ester, solvate, stereoisomer, tautomer, or prodrug thereof, wherein the first, second and third compounds are different.

Disclosed herein include methods for imparting resistance to an RNA virus to a cell(s). In some embodiments, the method comprises: contacting the cell(s) with (1) a first compound selected from the compounds listed in Tables 2-8, or a pharmaceutically acceptable salt, ester, solvate, stereoisomer, tautomer, or prodrug thereof, and (2) a second compound selected from the compounds listed in Tables 2-8, or a pharmaceutically acceptable salt, ester, solvate, stereoisomer, tautomer, or prodrug thereof, thereby imparting resistance to the RNA virus to the cell(s), wherein the first compound and the second compound are different. The method can comprise: contacting the cell(s) with (3) a third compound selected from the compounds listed in Tables 2-8, or a pharmaceutically acceptable salt, ester, solvate, stereoisomer, tautomer, or prodrug thereof, wherein the first, second and third compounds are different. The method can comprise: contacting a plurality of cells with the first compound, the second compound, and/or the third compound. The method can comprise: determining the infection rate of the plurality of cells after being contacted with the first compound, the second compound, and/or the third compound. In some embodiments, contacting the cell with the first compound, the second compound, and/or the third compound is in a subject. In some embodiments, contacting the cell with the first compound, the second compound, and/or the third compound occurs in vitro, ex vivo, and/or in vivo. In some embodiments, the cell expresses angiotensin-converting enzyme 2 (ACE2). The cell can be a lung cell, an enterocyte, an endothelial cell, an epithelial cell, a kidney cell, an arterial smooth muscle cell, a cell of the respiratory tract, or any combination thereof. The cell can be the cell of a subject. The cell can be in a subject.

The first compound, the second compound, and/or the third compound can be administered in a therapeutically or prophylactically effective amount. The first compound can be selected from the compounds listed in Table 2, and the second compound can be selected from the compounds listed in Table 3. The first compound can be selected from the compounds listed in Table 2, and the second compound can be selected from the compounds listed in Table 4. The first compound can be selected from the compounds listed in Table 2, and the second compound can be selected from the compounds listed in Table 5. The first compound can be selected from the compounds listed in Table 2, and the second compound can be selected from the compounds listed in Table 6. The first compound can be selected from the compounds listed in Table 2, and the second compound can be selected from the compounds listed in Table 7. The first compound can be selected from the compounds listed in Table 2, and the second compound can be selected from the compounds listed in Table 8. The first compound can be selected from the compounds listed in Table 3, and the second compound can be selected from the compounds listed in Table 4. The first compound can be selected from the compounds listed in Table 3, and the second compound can be selected from the compounds listed in Table 5. The first compound can be selected from the compounds listed in Table 3, and the second compound can be selected from the compounds listed in Table 6. The first compound can be selected from the compounds listed in Table 3, and the second compound can be selected from the compounds listed in Table 7. The first compound can be selected from the compounds listed in Table 3, and the second compound can be selected from the compounds listed in Table 8. The first compound can be selected from the compounds listed in Table 4, and the second compound can be selected from the compounds listed in Table 5. The first compound can be selected from the compounds listed in Table 4, and the second compound can be selected from the compounds listed in Table 6. The first compound can be selected from the compounds listed in Table 4, and the second compound can be selected from the compounds listed in Table 7. The first compound can be selected from the compounds listed in Table 4, and the second compound can be selected from the compounds listed in Table 8. The first compound can be selected from the compounds listed in Table 5, and the second compound can be selected from the compounds listed in Table 6. The first compound can be selected from the compounds listed in Table 5, and the second compound can be selected from the compounds listed in Table 7. The first compound can be selected from the compounds listed in Table 5, and the second compound can be selected from the compounds listed in Table 8. The first compound can be selected from the compounds listed in Table 6, and the second compound can be selected from the compounds listed in Table 7. The first compound can be selected from the compounds listed in Table 6, and the second compound can be selected from the compounds listed in Table 8. The first compound can be selected from the compounds listed in Table 7, and the second compound can be selected from the compounds listed in Table 8.

The first compound can be selected from the compounds listed in Table 2, the second compound can be selected from the compounds listed in Table 3, and the third compound can be selected from the compounds listed in Table 4. The first compound can be selected from the compounds listed in Table 2, the second compound can be selected from the compounds listed in Table 3, and the third compound can be selected from the compounds listed in Table 5. The first compound can be selected from the compounds listed in Table 2, the second compound can be selected from the compounds listed in Table 3, and the third compound can be selected from the compounds listed in Table 6. The first compound can be selected from the compounds listed in Table 2, the second compound can be selected from the compounds listed in Table 3, and the third compound can be selected from the compounds listed in Table 7. The first compound can be selected from the compounds listed in Table 2, the second compound can be selected from the compounds listed in Table 3, and the third compound can be selected from the compounds listed in Table 8. The first compound can be selected from the compounds listed in Table 2, the second compound can be selected from the compounds listed in Table 4, and the third compound can be selected from the compounds listed in Table 5. The first compound can be selected from the compounds listed in Table 2, the second compound can be selected from the compounds listed in Table 4, and the third compound can be selected from the compounds listed in Table 6. The first compound can be selected from the compounds listed in Table 2, the second compound can be selected from the compounds listed in Table 4, and the third compound can be selected from the compounds listed in Table 7. The first compound can be selected from the compounds listed in Table 2, the second compound can be selected from the compounds listed in Table 4, and the third compound can be selected from the compounds listed in Table 8. The first compound can be selected from the compounds listed in Table 2, the second compound can be selected from the compounds listed in Table 5, and the third compound can be selected from the compounds listed in Table 6. The first compound can be selected from the compounds listed in Table 2, the second compound can be selected from the compounds listed in Table 5, and the third compound can be selected from the compounds listed in Table 7. The first compound can be selected from the compounds listed in Table 2, the second compound can be selected from the compounds listed in Table 5, and the third compound can be selected from the compounds listed in Table 8. The first compound can be selected from the compounds listed in Table 2, the second compound can be selected from the compounds listed in Table 6, and the third compound can be selected from the compounds listed in Table 7. The first compound can be selected from the compounds listed in Table 2, the second compound can be selected from the compounds listed in Table 6, and the third compound can be selected from the compounds listed in Table 8. The first compound can be selected from the compounds listed in Table 2, the second compound can be selected from the compounds listed in Table 7, and the third compound can be selected from the compounds listed in Table 8. The first compound can be selected from the compounds listed in Table 3, the second compound can be selected from the compounds listed in Table 4, and the third compound can be selected from the compounds listed in Table 5. The first compound can be selected from the compounds listed in Table 3, the second compound can be selected from the compounds listed in Table 4, and the third compound can be selected from the compounds listed in Table 6. The first compound can be selected from the compounds listed in Table 3, the second compound can be selected from the compounds listed in Table 4, and the third compound can be selected from the compounds listed in Table 7. The first compound can be selected from the compounds listed in Table 3, the second compound can be selected from the compounds listed in Table 4, and the third compound can be selected from the compounds listed in Table 8. The first compound can be selected from the compounds listed in Table 3, the second compound can be selected from the compounds listed in Table 5, and the third compound can be selected from the compounds listed in Table 6. The first compound can be selected from the compounds listed in Table 3, the second compound can be selected from the compounds listed in Table 5, and the third compound can be selected from the compounds listed in Table 7. The first compound can be selected from the compounds listed in Table 3, the second compound can be selected from the compounds listed in Table 5, and the third compound can be selected from the compounds listed in Table 8. The first compound can be selected from the compounds listed in Table 3, the second compound can be selected from the compounds listed in Table 6, and the third compound can be selected from the compounds listed in Table 7. The first compound can be selected from the compounds listed in Table 3, the second compound can be selected from the compounds listed in Table 6, and the third compound can be selected from the compounds listed in Table 8. The first compound can be selected from the compounds listed in Table 3, the second compound can be selected from the compounds listed in Table 7, and the third compound can be selected from the compounds listed in Table 8. The first compound can be selected from the compounds listed in Table 4, the second compound can be selected from the compounds listed in Table 5, and the third compound can be selected from the compounds listed in Table 6. The first compound can be selected from the compounds listed in Table 4, the second compound can be selected from the compounds listed in Table 5, and the third compound can be selected from the compounds listed in Table 7. The first compound can be selected from the compounds listed in Table 4, the second compound can be selected from the compounds listed in Table 5, and the third compound can be selected from the compounds listed in Table 8. The first compound can be selected from the compounds listed in Table 4, the second compound can be selected from the compounds listed in Table 6, and the third compound can be selected from the compounds listed in Table 7. The first compound can be selected from the compounds listed in Table 4, the second compound can be selected from the compounds listed in Table 6, and the third compound can be selected from the compounds listed in Table 8. The first compound can be selected from the compounds listed in Table 4, the second compound can be selected from the compounds listed in Table 7, and the third compound can be selected from the compounds listed in Table 8. The first compound can be selected from the compounds listed in Table 5, the second compound can be selected from the compounds listed in Table 6, and the third compound can be selected from the compounds listed in Table 7. The first compound can be selected from the compounds listed in Table 5, the second compound can be selected from the compounds listed in Table 6, and the third compound can be selected from the compounds listed in Table 8. The first compound can be selected from the compounds listed in Table 5, the second compound can be selected from the compounds listed in Table 7, and the third compound can be selected from the compounds listed in Table 8. The first compound can be selected from the compounds listed in Table 6, the second compound can be selected from the compounds listed in Table 7, and the third compound can be selected from the compounds listed in Table 8.

The first, second and/or third compound can be in a pharmaceutical composition comprising one or more pharmaceutically acceptable excipients. The method can comprise: administering to the subject one or more additional therapeutic agents. The therapeutic agent can be selected from the group consisting of a nucleoside or a non-nucleoside analogue reverse-transcriptase inhibitor, a nucleotide analogue reverse-transcriptase inhibitor, a NS3/4A serine protease inhibitor, a NS5B polymerase inhibitor, and interferon alpha.

At least one of the one or more additional therapeutic agents can be administered to the subject before the administration of the first, second or third compound; after the administration of the first, second or third compound; or both. At least two of the first, second and third compounds can be co-administered in a single composition or in separate compositions to the subject. The first, second and third compounds can be co-administered in a single composition or in separate compositions to the subject. The first, second and/or third compound can be administered to the subject by intravenous administration, nasal administration, pulmonary administration, oral administration, parenteral administration, nebulization, or a combination thereof. The first, second and/or third compound can be aspirated into at least one lung of the subject.

At least one of the first, second and third compounds can be in a composition in the form of powder, pill, tablet, microtablet, pellet, micropellet, capsule, capsule containing microtablets, liquid, aerosols, or nanoparticles. At least one of the first, second and third compounds can be in a composition in a formulation for administration to the lungs. At least one of the first, second and third compounds can be administered to the subject once, twice, or three times a day. At least one of the first, second and third compounds can be administered to the subject once every day, every two days, or every three days. At least one of the first, second and third compounds can be administered to the subject over the course of at least two weeks, at least three weeks, at least four weeks, or at least five weeks.

The method can comprise: measuring the viral titer of the RNA virus in the subject before administering the first, second and/or the third compound to the subject, after administering the first, second and/or the third compound to the subject, or both. The viral titer can be lung bulk virus titer. In some embodiments, administrating the first, second and/or the third compound results in reduction of the viral titer of the RNA virus in the subject as compared to that in the subject before administration of the first, second and/or the third compound. The method can comprise: determining global virus distribution in the lungs of the subject. The method can comprise: measuring a neutrophil density within the lungs of the subject. In some embodiments, administering the first, second and/or the third compound results in reduction of the neutrophil density within the lungs of the subject as compared to that in the subject before administration of the first, second and/or the third compound. The method can comprise: measuring a total necrotized cell count within the lungs of the subject. Administering the first, second and/or the third compound can result in reduction of the total necrotized cell count in the subject as compared to that in the subject before administration of the first, second and/or the third compound. The method can comprise: measuring a total protein level within the lungs of the subject. In some embodiments, administering the first, second and/or the third compound results in reduction of the total protein level within the lungs of the subject as compared to that in the subject before administration of the first, second and/or the third compound.

In some embodiments, the method can comprise administering to the subject in need thereof one or more additional therapeutic agents (e.g., antiviral agents). The additional therapeutic agents (e.g., antiviral agents) can be co-administered to the subject with the composition. The additional therapeutic agents (e.g., antiviral agents) can be administered to the subject before the administration of the composition, after the administration of the composition, or both. The composition can comprise one or more additional therapeutic agents (e.g., antiviral agents).

The antiviral agent can be selected from the group consisting of a nucleoside or a non-nucleoside analogue reverse-transcriptase inhibitor, a nucleotide analogue reverse-transcriptase inhibitor, a NS3/4A serine protease inhibitor, a NS5B polymerase inhibitor, and interferon alpha.

As disclosed herein, co-administration of particular ratios and/or amounts of one or more compounds selected from the compounds listed in Tables 2-8 (or a pharmaceutically acceptable salt, ester, solvate, stereoisomer, tautomer, or prodrug thereof, e.g., a therapeutic agent) and one or more additional therapeutic agents (e.g., antiviral agents) can result in synergistic effects in preventing, delaying the onset of, or treating an infection, disease, or inflammatory effect caused by a RNA virus. These synergistic effects can be such that the one or more effects of the combination compositions are greater than the one or more effects of each component alone at a comparable dosing level, or they can be greater than the predicted sum of the effects of all of the components at a comparable dosing level, assuming that each component acts independently. The synergistic effect can be, be about, be greater than, or be greater than about, 5, 10, 20, 30, 50, 75, 100, 110, 120, 150, 200, 250, 350, or 500% better than the effect of treating a subject with one of the components alone, or the additive effects of each of the components when administered individually. The effect can be any of the measurable effects described herein. The composition comprising a plurality of components can be such that the synergistic effect is, for example, a reduction in lung inflammation and that lung inflammation is reduced to a greater degree as compared to the sum of the effects of administering each component, determined as if each component exerted its effect independently, also referred to as the predicted additive effect herein. For example, if a composition comprising component (a) yields an effect of a 20% reduction in lung inflammation and a composition comprising component (b) yields an effect of 50% reduction in lung inflammation, then a composition comprising both component (a) and component (b) would have a synergistic effect if the combination composition's effect on lung inflammation was greater than 70%.

A synergistic combination composition can have an effect that is greater than the predicted additive effect of administering each component of the combination composition alone as if each component exerted its effect independently. For example, if the predicted additive effect is 70%, an actual effect of 140% is 70% greater than the predicted additive effect or is 1 fold greater than the predicted additive effect. The synergistic effect can be at least, or at least about, 20, 50, 75, 90, 100, 150, 200 or 300% greater than the predicted additive effect. In some embodiments, the synergistic effect can be at least, or at least about, 0.2, 0.5, 0.9, 1.1, 1.5, 1.7, 2, or 3 fold greater than the predicted additive effect.

In some embodiments, the synergistic effect of the combination compositions can also allow for reduced dosing amounts, leading to reduced side effects to the subject and reduced cost of treatment. Furthermore, the synergistic effect can allow for results that are not achievable through any other treatments. Therefore, proper identification, specification, and use of combination compositions can allow for significant improvements in the reduction and prevention of lung inflammation.

The additional therapeutic agents provided herein can include antagonists of transient receptor potential cation channels, including but not limited to transient receptor potential ankyrin A1 (TRPA1) antagonists {e.g., camphor, isopentenyl pyrophosphate, A967079, GRC-17536, (4R)-1,2,3,4-tetrahydro-4-[3-(3-methoxypropoxy)phenyl]-2-thioxo-5H-indeno[1,2-d]pyrimidin-5-one, 2-amino-4-arylthiazole compounds disclosed in WO 2012/085662 A1, and specialized pro-resolving mediators (SPMs) (e.g., metabolites of polyunsaturated fatty acids [PUFAs])}, transient receptor potential vanilloid (TRPV) antagonists (e.g., TRPV1 antagonists [e.g., capsazepine, iodo-resiniferatoxin, AMG-517, GRC-6211, NGD-8243, SB-705498 and SPMs {e.g., PUFA metabolites}] and TRPV3 antagonists [e.g., SPMs {e.g., PUFA metabolites}]), and analogs, derivatives and salts thereof.

The additional therapeutic agents provided herein can include TRPV1 agonists that cause decrease in TRPV1 activity (desensitization) upon prolonged exposure of TRPV1 to the stimuli, including but not limited to capsaicin, camphor, carvacrol, menthol, methyl salicylate, resiniferatoxin, tinyatoxin, and analogs, derivatives and salts thereof.

The additional therapeutic agents provided herein can include antagonists of protease-activated receptors (PARs) and inhibitors of activating proteases, including but not limited to PAR1 antagonists (e.g., SCH-530,348), PAR2 antagonists {e.g., AY-117, ENMD-1068, ENMD-106836, GB-83, tetracyclines (e.g., doxycycline, minocycline and tetracycline), FSLLRY-NH₂ (PAR-3888-PD, Ac-FSLLRY-NH₂ and anti-PAR2 antibodies (e.g., SAM-11 [SC-13504], P2pal-21 and P2pal-2135}, PAR4 antagonists {e.g, ethanol, YD-3, statins atorvastatin, cerivastatin, fluvastatin, lovastatin, pitavastatin, pravastatin, rosuvastatin and simvastatin), pepducin P4 pal-10, pepducin P4 pal-15, trans-cinnamoyl-APGKF-NH₂, trans-cinnamoyl-YPGKF-NH₂, and anti-PAR4 antibodies (e.g., C-19 and SC-1249)}, inhibitors of serine proteases {e.g., benzamidine hydrochloride, 4-iodo-1-benzothiophene-2-carboximidamide hydrochloride (inhibits trypsin and tryptase), inhibitors of kallikreins (e.g., camostat, nafamostat, gabexate, ecallantide and α₁-inhibitor), trypsin inhibitors tosyllysine chloromethyl ketone [TLCK]hydrochloride, α₁-antitrypsin, aprotinin, ovomucin and soybean trypsin inhibitor), and tryptase inhibitors (e.g., camostat, nafamostat, gabexate, AMG-126737 and APC-366)}, inhibitors of cysteine proteases {e.g., E-64 (non-specific inhibitor), JNJ-10329670, RWJ-445380, cystatin C, leupeptin, stefin A, stefin B, testican-1, chloroquine, fluoromethyl ketone, naphthalene endoperoxide (inhibits cathepsin B, L and S), CA-074 (inhibits cathepsin B), odanacatib (MK-0822, inhibits cathepsin K), CLIK-148 and CLIK-195 (inhibit cathepsin L), and CLIK-60 and E-6438 (inhibit cathepsin S)}, and analogs, derivatives, fragments and salts thereof,

The additional therapeutic agents provided herein can include antagonists of endothelin receptors, including but not limited to selective endothelin A receptor (ETAR) antagonists {e.g., ambrisentan, atrasentan, sitaxentan, zibotentan, BQ-123, 4-amino-N-(3,4-dimethylisoxazol-5-yl)benzenesulfonamide; (2R)-2-[[(2R)-2-[[(2 S)-2-(azepane-1-carbonylamino)-4-methylpentanoyl]amino]-3-(1-formylindol-3-yl)propanoyl]amino]-3-(1H-indol-3-yl)propanoic acid; 3-benzodioxol-5-yl)-1-[2-(dibutylamino)-2-oxoethyl]-2-(4-methoxyphenyl)pyrrolidine-3-carboxylic acid; (2R,3R,4S)-4-(1,3-benzodioxol-5-yl)-1-[2-(dibutylamino)-2-oxoethyl]-2-(4-methoxyphenyl)pyrrolidine-3-carboxylic acid; (2R,3R,4S)-4-(1,3-benzodioxol-5-yl)-1[2-(dibutylamino)-2-oxoethyl]-2-(2-methoxyphenyl)pyrrolidine-3-carboxylic acid; 3-(1,3-benzodioxol-5-yl)-5-hydroxy-5-(4-methoxyphenyl)-4-[(3,4,5-trimethoxyphenyl)methyl]furan-2-one; 2-(1,3-benzodioxol-5-yl)-4-(4-methoxyphenyl)-4-oxo-3-[(3,4,5-trimethoxyphenyl)methyl]but-2-enoate; 5-(4-bromophenyl)-6-[2-(5-bromopyrimidin-2-yl)oxyethoxy]-N-(propylsulfamoyl)pyrimidin-4-amine; 4-tert-butyl-N-[6-(2-hydroxyethoxy)-5-(2-methoxyphenoxy)-2-(pyrimidin-2-yl)pyrimidin-4-yl]benzenesulfonamide; [(7R)-5-chloro-3-[(1E,3E,5S)-3,5-dimethylhepta-1,3-dienyl]-7-methyl-6,8-dioxoisochromen-7-yl]acetate; N-(4-chloro-3-methyl-1,2-oxazol-5-yl)-2-[2-(6-methyl-2H-1,3-benzodioxol-5-yl)acetyl]thiophene-3-sulfonamide; (2S)-2-(4,6-dimethoxypyrimidin-2-yl)oxy-3-methoxy-3,3-diphenylpropanoic acid; (2S)-2-[(4,6-dimethylpyrimidin-2-yl)oxyl-3-methoxy-3,3-diphenylpropanoic acid; N-[6-(2-hydroxyethoxy)-5-(2-methoxyphenoxy)-2-[2-(2H-tetrazol-5-yl)pyridin-4-yl]pyrimidin-4-yl]-5-methylpyridine-2-sulfonamide; N-[6-(2-hydroxyethoxy)-5-(2-methoxyphenoxy)-2-[2-(2H-tetrazol-5-yl)pyridin-4-yl]pyrimidin-4-yl]-5-propan-2-ylpyridine-2-sulfonamide; 6-(2-hydroxy-ethoxy)-5-(2-methoxyphenoxy)-2-[2-(1,2,3-triaza-4-azanidacyclopenta-2,5-dien-5-yl)pyridin-4-yl]pyrimidin-4-yl]-(5-methylpyridin-2-yl)sulfonylazanide; 2-[(3R,6R,9S,12R,15S)-6-(1H-indol-3-ylmethyl)-9-(2-methylpropyl)-2,5,8,11,14-pentaoxo-12-propan-2-yl-1,4,7,10,13-pentazabicyclo[13.3.0]octadecan-3-yl]acetic acid; N-[6-methoxy-5-(2-methoxyphenoxy)-2-pyridin-4-ylpyrimidin-4-yl]-5-methylpyridisulfonamide; N-(3-methoxy-5-methylpyrazin-2-yl)-2-[4-(1,3,4-oxadiazol-2-yl)phenyl]pyridine-3-sulfonamide; and N-[5-(2-methoxyphenoxy)-2-pyridin-4-yl-6-(trideuteriomethoxy)pyrimidine-4-yl]-5-methylpyridine-2-sulfonamide}, selective endothelin B receptor (ETBR) antagonists (e.g., A-192621 and BQ-788), dual ETAR/ETBR antagonists (e.g., bosentan, macitentan and tezosentan), and analogs, derivatives and salts thereof.

The additional therapeutic agents provided herein can include inhibitors of Toll-like receptors (TLRs), including, but not limited to TIR7/non-TLR9 inhibitors (e.g., ODN 2087, ODN 20958 and ODN 20959), dual TLR7/TLR9 inhibitors (e.g., chloroquine, hydroxychloroquine, quinacrine, AT791, DV056, E6446, IMO-3100, IMO-8400 and ODN 2088), and analogs, derivatives, fragments and salts thereof.

The additional therapeutic agents provided herein can include inhibitors of mitogen-activated protein (MAP) kinases, including but not limited to p38 MAP kinase inhibitors {e.g., BMS-582949, CPSI-2364, 4-(4-fluorophenyl)-2-(4-hydroxyphenyl)-5-(4-pyridyl)-1H-imidazole, trans-4-[4-(4-fluorophenyl)-5-(2-methoxy-4-pyrimidinyl)-1H-imidazole-1-yl-]cyclohexanol, and 4-(4-fluorophenyl)-2-(4-methylsulfinylphenyl)-5-(4-pyridyl)-1H-imidazole}, and analogs, derivatives and salts thereof.

The additional therapeutic agents provided herein can include inhibitors of mitogen-activated protein kinase kinases (MEKs), including but not limited to MEK 1 inhibitors {e.g., N-[3-[5-(2-aminopyrimidin-4-yl)-2-tert-butyl-1,3-thiazol-4-yl]-2-fluorophenyl]-2,6-difluorobenzenesulfonamide; N-[3-[5-(2-aminopyrimidin-4-yl)-2-tert-butyl-1,3-thiazol-4-yl]-2-fluorophenyl]-2,6-difluorobenzenesulfonamide, methanesulfonic acid; 6-(4-bromo-2-chloroanilino)-7-fluoro-N-(2-hydroxyethoxy)-3-methylbenzimidazole-5-carboxamide; 5-bromo-N-(2,3-dihydroxypropoxy)-3,4-difluoro-2-(2-fluoro-4-iodoanilino)benzamide; 6-(4-bromo-2-fluoroanilino)-7-fluoro-N-(2-hydroxyethoxy)-3-methylbenzimidazole-5-carboxamide; 2-[4-[(2-butyl-4-oxo-1,3-diazaspiro[4.4]non-1-en-3-yl)methyl]-2-(ethoxymethyl)phenyl]-N-(3,4-dimethyl-1,2-oxazol-5-yl)benzenesulfonamide; 2-[4-[(2-butyl-4-oxo-1,3-diazaspiro[4.4]non-1-en-3-yl)methyl]-2-(ethoxymethyl)phenyl]-N-(4,5-dimethyl-1,2-oxazol-3-yl)benzenesulfonamide; 2-[4-[(2-butyl-4-oxo-1,3-diazaspiro [4.4]non-1-en-3-yl)methyl]-2-propylphenyl]-N-(4,5-dimethyl-1,2-oxazol-3-yl)benzenesulfonamide; 2-(2-chloro-4-iodoanilino)-N-(cyclopropylmethoxy)-3,4-difluorobenzamide; N-[3-[3-cyclopropyl-5-(2-fluoro-4-iodoanilino)-6,8-dimethyl-2,4,7-trioxopyrido[4,3-d]pyrimidin-1-yl]phenyl]acetamide; 3,4-difluoro-2-(2-fluoro-4-iodoanilino)-N-(2-hydroxyethoxy)-5-[(3-oxooxazinan-2-yl)methyl]benzamide; N-[3,4-difluoro-2-(2-fluoro-4-iodoanilino)-6-methoxyphenyl]-[(2S)-2,3-dihydroxypropyl]cyclopropane-1-sulfonamide; [3,4-difluoro-2-(2-fluoro-4-iodoanilino)phenyl]-[3-hydroxy-3-[(2S)-piperidin-2-yl]azetidin-1-yl]methanone; N-[(2R)-2,3-dihydroxypropoxy]-3,4-difluoro-2-(2-fluoro-4-iodoanilino)benzamide; (2S,3S)-2-[(4R)-4-[4-[(2R)-2,3-dihydroxypropoxy]phenyl]-2,5-dioxoimidazolidin-1-yl]-N-(2-fluoro-4-iodophenyl)-3-phenylbutanamide; 3-[(2R)-2,3-dihydroxypropyl]-6-fluoro-5-(2-fluoro-4-iodoanilino)-8-methylpyrido[2,3-(1]pyrimidine-4,7-dione; N-[(2S)-2,3-dihydroxypropyl]-3-(2-fluoro-4-iodoanilino)pyridine-4-carboxamide, and 2-(2-fluoro-4-iodoanilino)-N-(2-hydroxyethoxy)-1,5-dimethyl-6-oxopyridine-3-carboxamide}, and analogs, derivatives and salts thereof.

The additional therapeutic agents provided herein can include inhibitors of calcitonin gene-related peptide (CGRP) or receptor therefor or the production thereof, including but not limited to CORP receptor antagonists (e.g., olcegepant, telcagepant, ubrogepant, eptinezumab [ALD-403], AMG-334, LY-2951742 and TEV-48125), and analogs, derivatives, fragments and salts thereof.

The additional therapeutic agents provided herein can include inhibitors of gastrin-releasing peptide (GRP) or the receptor therefor (GRPR, aka bombesin receptor 2 [BBR2]) or the production thereof, including but not limited to CRPR antagonists (e.g.; RC-3095), and analogs, derivatives and salts thereof.

The additional therapeutic agents provided herein can include inhibitors of nerve growth factor (NGF) or receptors therefor tropomyosin kinase receptor A [TrkA]) or the production thereof, including but not limited to NGF inhibitors (e.g., fulranumab and tanezumab), NGF receptor inhibitors (e.g., TrkA inhibitors such as A0879, CT327 and K252a), and analogs, derivatives, fragments and salts thereof.

The additional therapeutic agents provided herein can include inhibitors of neurotensin or receptors therefor (e.g., neurotensin receptor 1 [NTSR1], NTSR2 and so 1) or the production thereof, including but not limited to selective NTSR1 antagonists (e.g., SR-48,692), selective NTSR2 antagonists (e.g., levocabastine), unselective receptor antagonists (e.g., SR-142,948), and analogs, derivatives and salts thereof.

The additional therapeutic agents provided herein can include inhibitors of somatostatin or receptors therefor (e.g., somatostatin receptors [SSTRs] 1 to 5) or the production thereof, including but not limited to selective SSTR2 antagonists (e.g., CYN 154806), selective SSTRS antagonists (e.g., BIM 23056), unselective SSTR antagonists (e.g., cyclosomatostatin), and analogs, derivatives, fragments and salts thereof.

The additional therapeutic agents provided herein can include inhibitors of vasoactive intestinal peptide (VIP) or receptors therefor (e.g., VIPR1 and VIPR2) or the production thereof, including but not limited to VIP receptor antagonists {e.g., PG 97-269, ViPhyb, VIP(6-28)-NH₂, [p-Cl-D-Phe⁶, Leu¹⁷]VIP-NH₂, [Ac-His¹, D-Phe², Lys¹⁵, Arg¹⁶]VIP(3-7)GRF(8-27)-NH₂, and [Ac-Tyr¹, D-Phe²]GRF(1-29)-NH₂}, and analogs, derivatives, fragments and salts thereof.

The additional therapeutic agents provided herein can include inhibitors of bradykinin or receptors therefor (e.g., B1 and B2) or the production thereof, including but not limited to bradykinin inhibitors (e.g., aloe, bromelain and polyphenols), bradykinin receptor B2 antagonists (e.g., icatibant and FR-173657), inhibitors of kallikreins (e.g., ecallantide, camostat, nafamostat, gabexate and C1-inhibitor), and analogs, derivatives and salts thereof.

The additional therapeutic agents provided herein can include inhibitors of corticotropin-releasing hormone (CRH, aka corticoliberin) or receptors therefor (e.g., CRHR1 and CRHR2) or the production thereof, including but not limited to CRHR1 antagonists (e.g., antalarmin, pexacerfont, CP-154,526 LWH-234, NBI-27914 and R-121,919), CRHR2 antagonists (e.g., astressin-B), and analogs, derivatives and salts thereof.

The additional therapeutic agents provided herein can include antihistamines, including but not limited to antihistamines that inhibit action at the histamine H₁ receptor (e.g., acrivastine, antazoline, astemizole, azatadine, azelastine, bepotasiine, bilastine, bromodiphenhydramine, brompheniramine, buclizine, carbinoxamine, cetirizine, chlorcyclizine, chlorodiphenhydramine, chlorpheniramine, chlorpromazine, chloropyramine, cidoxepin, clemastine, cyclizine, cyproheptadine, desloratadine, dexbrompheniramine, dexchlorpheniramine, dimenhydrinate, dimetindene, diphenhydramine, doxepin, doxylamine, ebastine, embramine, esmirtazapine [(S)-(+)-enantiomer of mirtazapine], fexofenadine, hydroxyzine, ketotifen, levocabastine, levocetirizine, loratadine, meclozine mepyramine, mirtazapine, mizolastine, olopatadine, orphenadrine, phenindamine, pheniramine, phenyltoloxamine, promethazine, pyrilamine, quetiapine, quifenadine, rupatadine, terfenadine, trimeprazine tripelennamine and triprolidine), antihistamines that inhibit action at the histamine H₃ receptor (e.g., betahistine, burimamide, ciproxifan, clobenpropit, conessine, failproxifan, impentamine, iodophenpropit, irdabisant, pitolisant, thioperamide, A-349,821, ABT-239 and VUF-568), antihistamines that inhibit action at the histamine H₄ receptor (e.g., clobenpropit, thioperamide, A943931, A987306, JNJ-7777120, VUF-6002 and ZPL-389), and analogs, derivatives and salts thereof.

The additional therapeutic agents provided herein can include inhibitors of phospholipase A2 (e.g., secreted and cytosolic PLA2), including but not limited to arachidonyl trifluoromethyl ketone, bromoenol lactone, chloroquine, cytidine 5-diphosphoamines, darapladib, quinacrine, vitamin E, RO-061606, ZPL-521, lipocortins (annexins), and analogs, derivatives, fragments and salts thereof.

The additional therapeutic agents provided herein can include inhibitors of pro-inflammatory prostaglandins (e.g., prostaglandin E2) or receptors therefor or the production thereof, including but not limited to non-steroidal anti-inflammatory drugs (NSAIDs) (e.g., non-selective COX-1/COX-2 inhibitors such as aspirin and selective COX-2 inhibitors such as coxibs), glucocorticoids, cyclopentenone prostaglandins (e.g., prostaglandin J2 [PGJ2], Δ12-PGJ2 and 15-deoxy-Δ12,14-PGJ2), and analogs, derivatives and salts thereof, inhibitors of leukotrienes or receptors therefor or the production thereof, including but not limited to leukotriene receptor antagonists (e.g., cinalukast, gemilukast, iralukast, montelukast, pranlukast, tomelukast, verlukast, zafirlukast, CP-199330, HAMI-3379, ICI-198615 and MK-571), 5-lipoxygenase inhibitors (e.g., baicalein, caffeic acid, curcumin, hyperforin, meclofenamic acid, meclofenamate sodium, zileuton and MK-886), and analogs, derivatives and salts thereof.

The additional therapeutic agents provided herein can include mast cell stabilizers, including but not limited to cromoglicic acid (cromolyn), ketotifen, methylxanthines, nedocromil, olopatadine, omalizumab, pemirolast, quercetin. β₂-adrenoreceptor agonists {including short-acting β₂-adrenergic agonists (e.g., bitolterol, fenoterol, isoprenaline [isoproterenol], levosalbutamol [levalbuterol], orciprenaline [metaproterenol], pirbuterol, procaterol, ritodrine, salbutamol [albuterol] and terbutaline), long-acting β₂-adrenergic agonists arformoterol, bambuterol, clenbuterol, formoterol and salmeterol), and ultralong-acting β₂-adrenergic agonists (e.g., carmoterol, indacaterol, milveterol, olodaterol and vilanterol)}, and analogs, derivatives and salts thereof.

The additional therapeutic agents provided herein can include Janus kinase (JAX) inhibitors, including, but not limited to JAK1 inhibitors (e.g., GLPG0634 and GSK2586184). JAK2 inhibitors (e.g., lestaurtinib, pacritinib, CYT387 and TG101348), JAK3 inhibitors (e.g., ASP-015K, 8348 and VX-509), dual JAK1/JAK2 inhibitors (e.g., baricitinib and ruxolitinib), dual JAK1/JAK3 inhibitors (e.g., tofacitinib), and analogs, derivatives and salts thereof.

The additional therapeutic agents provided herein can include immunomodulators, including but not limited to imides (e.g., thalidomide, lenalidomide, pomalidomide and apremilast), xanthine derivatives (e.g., lisofylline, pentoxifylline and propentofylline), and analogs, derivatives and salts thereof.

The additional therapeutic agents provided herein can include immunosuppressants, including but not limited to glucocorticoids, antimetabolites (e.g., hydroxyurea [hydroxycarbamide], antifolates [e.g., methotrexate], and purine analogs [e.g., azathioprine, mercaptopurine and thioguanine]), calcineurin inhibitors (e.g, ciclosporin [cyclosporine A], pimecrolimus and tacrolimus), inosine-5′-monophosphate dehydrogenase (IPMPDH) inhibitors (e.g., mycophenolic acid and derivatives thereof [e.g., mycophenolate sodium and mycophenolate mofetil]), mechanistic/mammalian target of rapamycin (mTOR) inhibitors (e.g., rapamycin [sirolimus], deforolimus [ridaforolimus], everolimus, temsirolimus, umirolimus [biolimus A9], zotarolimus and RTP-801), modulators of sphingosine-1-phosphate receptors (e.g., SIPR1) (e.g., fingolimod), serine C-palmitoyltransferase inhibitors (e.g., myriocin), and analogs, derivatives and salts thereof.

The additional therapeutic agents provided herein can include corticosteroids/glucocorticoids, including but not limited to hydrocortisone types (e.g., cortisone and derivatives thereof [e.g., cortisone acetate], hydrocortisone and derivatives thereof [e.g., hydrocortisone acetate, hydrocortisone-17-aceponate, hydrocortisone-17-buteprate, hydrocortisone-17-butyrate and hydrocortisone-17-valerate], prednisolone, methylprednisolone and derivatives thereof [e.g., methylprednisolone aceponate], prednisone, and tixocortol and derivatives thereof [e.g., tixocortol pivalate]), betamethasone types (e.g., betamethasone and derivatives thereof [e.g., betamethasone dipropionate, betamethasone sodium phosphate and betamethasone valerate], dexamethasone and derivatives thereof [e.g., dexamethasone sodium phosphate], and fluocortolone and derivatives thereof [e.g., fluocortolone caproate and fluocortolone pivalate]), halogenated steroids (e.g., alclometasone and derivatives thereof [e.g., alclometasone dipropionate], beclometasone and derivatives thereof [e.g., beclometasone dipropionate], clobetasol and derivatives thereof [e.g., clobetasol-17-propionate], clobetasone and derivatives thereof [e.g., clobetasone-17-butyrate], desoximetasone and derivatives thereof [e.g., desoximetasone acetate], diflorasone and derivatives thereof [e.g., diflorasone diacetate], diflucortolone and derivatives thereof [e.g., diflucortolone valerate], fluprednidene and derivatives thereof [e.g., fluprednidene acetate], fluticasone and derivatives thereof [e.g., fluticasone propionate], halobetasol [ulobetasol] and derivatives thereof [e.g., halobetasol proprionate], halometasone and derivatives thereof [e.g., halometasone acetate], and mometasone and derivatives thereof [e.g., mometasone furoate]), acetonides and related substances (e.g., amcinonide, budesonide, ciclesonide, desonide, fluocinonide, fluocinolone acetonide, flurandrenolide [flurandrenolone or fludroxycortide], halcinonide, triamcinolone acetonide and triamcinolone alcohol), carbonates (e.g., prednicarbate), and analogs, derivatives and salts thereof.

The additional therapeutic agents provided herein can include inhibitors of pro-inflammatory cytokines or receptors therefor, including but not limited to inhibitors of (e.g., antibodies to) tumor necrosis factor-alpha (TNF-α) (e.g, adalimumab, certolizumab pegol, golimumab, infliximab, etanercept, bupropion and ART-621), inhibitors of (e.g., antibodies to) pro-inflammatory interferons (e.g., interferon-alpha [IFN-α]) or receptors therefor, inhibitors of (e.g., antibodies to) pro-inflammatory interleukins or receptors therefor (e.g., IL-1 [e.g., IL-1a and IL-1β] or IL-1R [e.g., EBI-005 {isunakinra}], IL-2 or IL-2R [e.g., basiliximab and daclizumab], IL-4 or IL-4R [e.g., dupilumab], IL-5 [e.g., mepolizumab] or IL-5R, IL-6 [e.g., clazakizumab, elsilimomab, olokizumab, siltuximab and sirukumab] or IL-6R [e.g., sarilumab and tocilizumab], IL-8 or IL-8R, IL-12 [e.g., briakinumab and ustekinumab] or IL-12R, IL-13 or IL-13R, IL-15 or IL-15R, IL-17 [e.g., ixekizumab and secukinumab] or IL-17R [e.g., brodalumab], IL-18 or IL-18R, IL-20 [e.g., the antibody 7E] or IL-20R, IL-22 [e.g., fezakinumab] or IL-22R, IL-23 [e.g., briakinumab, guselkumab, risankizumab, tildrakizumab SCH-9002221, ustekinumab and BI-655066] or IL-23R, IL-31 or IL-31R [e.g., anti-IL-31 receptor A antibodies such as nemolizumab], IL-33 or IL-33R, and IL-36 or IL-36R), and analogs, derivatives, fragments and salts thereof.

The additional therapeutic agents provided herein can include inhibitors of the production of pro-inflammatory cytokines or receptors therefor, including but not limited to inhibitors of the production of TNF-α (e.g., myxoma virus M013 protein, Yersinia YopM, protein, glucocorticoids, immunomodulatory imides, PDE4 inhibitors, p38 MAP kinase inhibitors, inhibitors of TLRs such as TLR7 and TLR9, scrim protease inhibitors [e.g., gabexate and nafamostat], and prostacyclin, carbacyclin and analogs and derivatives thereof [e.g., beraprost, cicaprost, ciprosten, eptaloprost, iloprost and treprostinil]), IFN-α (e.g., alefacept and inhibitors of TLRs such as TLR7 and TLR9), IL-1 (e.g., IL-1α, and IL-1β) (e.g., M013 protein, YopM protein, nafamostat, prostacyclin, glucocorticoids, TNF-α inhibitors, inhibitors of TLRs such as TLR7 and TLR9, and PAR1 antagonists), IL-2 (e.g., glucocorticoids, calcineurin inhibitors and PDE4 inhibitors), IL-4 (e.g., glucocorticoids and serine protease inhibitors [e.g., gabexate and nafamostat]), IL-5 (e.g., glucocorticoids), IL-6 M013 protein, nafamostat, prostacyclin, tranilast, glucocorticoids, immunomodulatory imides, TNF-α inhibitors, and inhibitors of TLRs such as TLR7 and TLR9), IL-8 alefacept, glucocorticoids and PAR2 antagonists [e.g., tetracyclines]), IL-12 (e.g., apilimod, YopM protein, PDE4 inhibitors, and inhibitors of TLRs such as TLR7 and TLR9), IL-15 (e.g., YopM protein), IL-17 (e.g., protein kinase C [PKC] inhibitors such as sotrastaurin), IL-18 (e.g., MOD protein and YopM protein), and IL-23 (e.g., apilimod, alefacept and PDE4 inhibitors), and analogs, derivatives, fragments and salts thereof.

The additional therapeutic agents provided herein can include other kinds of anti-inflammatory agents, including but not limited to inhibitors of pro-inflammatory transcription factors e.g., inhibitors of NE-κB [e.g., nafamostat, M013 protein, penetranin, (−)-DHMEQ, IT-603, IT-901 and PBS-1086] and inhibitors of STAT [signal transducer and activator of transcription] proteins [e.g., JAK1, JAK2 and JAK3 inhibitors]), antagonists of the prostaglandin D₂ receptor (DP₁) or/and the chemoattractant receptor homologous molecule expressed on TH₂ cells (CRTH2) (e.g., TS-022), phosphodiesterase (PDE) inhibitors (e.g., PDE4 inhibitors such as apremilast, cilomilast, ibudilast, piclamilast, roflumilast, crisaborole, diazepam, luteolin, mesembrenone, rolipram, AN2728 and E6005), IgE inhibitors (e.g., anti-IgE antibodies such as omalizumab), myeloperoxidase inhibitors (e.g., dapsone), specialized pro-resolving mediators (SPMs) (e.g., metabolites of polyunsaturated fatty acids such as lipoxins, resolvins [including resolvins derived from 5Z,8Z,11Z,14Z,17Z-eicosapentaenoic acid {EPA}, resolvins derived from 4Z,7Z,10Z,13Z,16Z,19Z-docosahexaenoic acid {DHA}, and resolvins derived from 7Z, 10Z,13Z,16Z,19Z-docosahexaenoic acid {n-3 DPA}], protectins/neuroprotectins [including DHA-derived protectins/neuroprotectins and n-3 DPA-derived protectins/neuroprotectins], maresins [including DHA-derived maresins and n-3 DPA-derived maresins], n-3 DPA metabolites, n-6 DPA {4Z,7Z,10Z,13Z,16Z-docosapentaenoic acid} metabolites, oxo-DHA metabolites, oxo-DPA metabolites, docosahexaenoyl ethanolamide metabolites, cyclopentenone prostaglandins [e.g., Δ12-PGJ2 and 15-deoxy-Δ12,14-PGJ2], and cyclopentenone isoprostanes [e.g., 5,6-epoxyisoprostane A2 and 5,6-epoxyisoprostane E2]), disease-modifying antirheumatic drugs (DMARDs, e.g., sulfasalazine and mesalazine [5-aminosalicylic acid]), anti-allergic agents (e.g., antihistamines, inhibitors of leukotrienes or receptors therefor or the production thereof, mast cell stabilizers, glucocorticoids, epinephrine [adrenaline] and tranilast), ultraviolet radiation (e.g., ultraviolet A and B), and analogs, derivatives, fragments and salts thereof.

The additional therapeutic agents provided herein can include antagonists of serotonin receptors, including but not limited to 5-HT₂ antagonists (e.g., clozapine, cyproheptadine ketanserin, pizotifen [pizotyline] and quetiapine), 5-HT₃ antagonists (e.g., alosetron, bemesetron, cilansetron, dolasetron, granisetron, ondansetron, palonosetron, ricasetron, tropanserin, tropisetron, zatosetron, mirtazapine, esmirtazapine and substances present in ginger [e.g., galanolactone, gingerols and shogaols]), and analogs, derivatives and salts thereof.

The additional therapeutic agents provided herein can include antagonists of muscarinic acetylcholine receptors (e.g., M1 to M5), including but not limited to aclidinium, atropine, benzatropine, biperiden, chlorpheniramine, cyclopentolate, darifenacin, dicyclomine, dimenhydrinate, diphenhydramine, doxepin, doxylamine, flavoxate, glycopyrrolate, hyoscyamine, ipratropium, orphenadrine, oxitropium, oxybutynin, pirenzepine, procyclidine, scopolamine (hyoscine), solifenacin, tolterodine, tiotropium, trihexyphenidyl, tropicamide, tricyclic antidepressants, and analogs, derivatives and salts thereof.

Examples of non-steroidal anti-inflammatory drugs (NSAIDs) the can be employed with the compounds provided herein include, but are not limited to: acetic acid derivatives, such as aceclofenac, bromfenac, diclofenac, etodolac, indomethacin, ketorolac, nabumetone, sulindac, sulindac sulfide, sulindac sulfone and tolmetin; anthranilic acid derivatives (fenamates), such as flufenamic acid, meclofenamic acid, mefenamic acid and tolfenamic acid; enolic acid derivatives (oxicams), such as droxicam, isoxicam, lornoxicam, meloxicam, piroxicam and tenoxicam; propionic acid derivatives, such as fenoprofen, flurbiprofen, ibuprofen, dexibuprofen, ketoprofen, dexketoprofen, loxoprofen, naproxen and oxaprozin; salicylates, such as diflunisal, salicylic acid, acetylsalicylic acid (aspirin), choline magnesium trisalicylate, and salsalate; COX-2-selective inhibitors, such as apricoxib, celecoxib, etoricoxib, firocoxib, fluorocoxibs (e.g., fluorocoxibs A-C), lumiracoxib, mavacoxib, parecoxib, rofecoxib, tilmacoxib (JTE-522), valdecoxib, 4-O-methylhonokiol, niflumic acid, DuP-697, CG100649, GW406381, NS-398, SC-58125, benzothieno[3,2-d]pyrimidin-4-one sulfonamide thio-derivatives, and COX-2 inhibitors derived from Tribulus terrestris; other kinds of NSAIDs, such as monoterpenoids (e.g., eucalyptol and phenols [e.g., carvacrol]), anilinopyridinecarboxylic acids (e.g., clonixin), sulfonanilides (e.g., nimesulide), and dual inhibitors of lipooxygenase (e.g., 5-LOX) and cyclooxygenase (e.g., COX-2) [e.g., chebulagic acid, licofelone, 2-(3,4,5-trimethoxyphenyl)-4-(N-methylindol-3-yl)thiophene, and di-tert-butylphenol-based compounds (e.g., DTPBHZ, DTPINH, DTPNHZ and DTPSAL)]; and analogs, derivatives and salts thereof.

The one or more antiviral agents and/or the one or more additional therapeutic agents can one or more of the following: Gimsilumab, an anti-granulocyte-macrophage colony stimulating factor monoclonal antibody, a non-viral gene therapy producing monoclonal antibodies, EB05, a non-steroidal anti-inflammatory molecule (sPLA2 inhibitor), Opdivo (nivolumab), a PD-1 blocking antibody, IC14, a recombinant chimeric anti-CD14 monoclonal antibody, avastin (bevacizumab), a vascular endothelial growth factor inhibitor, a PD-1 blocking antibody, Thymosin, meplazumab, an anti-CD147 antibody, an antibody combination REGN-COV2 (REGN10933+REGN10987) against the spike protein MEDI3506, a monoclonal antibody targeting interleukin 33, OmniChicken platform antibodies, antibodies from recovered COVID-19 patients, Antibody 47D11, Polyclonal hyperimmune globulin (H-IG), LY-CoV555 antibody, otilimab, an anti-granulocyte macrophase colony-stimulating factor (GM-CSF) antibody, LY3127804, an anti-Angiopoietin 2 (Ang2) antibody, a CXC10 antagonist, polyclonal hyperimmune globulin (H-IG), Octagam, intravenous Immunoglobulin (IVIG), single domain antibodies (sdAbs), an engineered monoclonal antibody derived from camelids, a super-antibody or antibody cocktail to target potential mutations of SARS-CoV-2, AiRuiKa (camrelizumab), an anti-programmed cell death protein (PD-1) antibody, Linked nanobody antibody, antibodies from recovered COVID-19 patients, OmniRat platform antibodies, Soliris (eculizumab), a complement inhibitor, CT-P59, Ultomiris (ravulizumab-cwvz), rCIG (recombinant anti-coronavirus 19 hyperimmune gammaglobulin), VIR-7831, VIR-7832, Gamifant (emapalumab), an anti-interferon gamma antibody, leronlimab (PRO 140), an CCR5 antagonist, polyclonal hyperimmune globulin (H-IG), Sylvant (siltuximab), an interleukin-6 targeted monoclonal antibody, Actemra (tocilizumab), an interleukin-6 receptor antagonist, Kevzara (sarilumab), an interleukin-6 receptor antagonist, purified ovine immunoglobulin from immunized sheep, lenzilumab, an anti-granulocyte-macrophage colony stimulating factor antibody, Ilaris (canakinumab), an interleukin-1beta blocker, JS016 antibody, TJM2 (TJ003234), an anti-granulocyte-macrophage colony stimulating factor antibody, COVI-SHIELD antibody cocktail, an antibody targeting the S protein, COVID-EIG plasma, SAB-185, polyclonal hyperimmune globulin (H-IG), IFX-1, an anti-C5a antibody, CERC-002, an anti-LIGHT monoclonal antibody, Remsima (infliximab), an anti-TNF antibody, TY027, a monoclonal antibody targeting SARS-CoV-2, IgY-110, an anti-CoV-2 antibody (nasal spray application), mavrilimumab, an anti-granulocyte-macrophase colony-stimulating factor receptor-alpha monoclonal antibody, BDB-100, monocloncal anti-C5a antibody, TZLS-501, an anti-interleukin-6 receptor monoclonal antibody, itolizumab, anti-CD6 IgG1 monoclonal antibody, GC5131A, BTL-tml, galidesivir, emetine hydrochloride, DAS181, recombinant sialidase (nebulized), Favilavir/Favipiravir/T-705/Avigan, Vicromax, ISR-50, Levovir (clevudine), AB001, EIDD-2801, an oral ribonucleoside analog, ASC09, an HIV protease inhibitor, Tamiflu (oseltamivir), a neuraminidase inhibitor, Truvada, emtricitabine, tenofovir, a HIV-1 nucleoside analog reverse transcriptase inhibitor, Virazole, ribavirin for inhalation solution, AT-527, an oral purine nucleotide prodrug, Ganovo (danoprevir), a hepatitis C virus NS3 protease inhibitor, ritonavir, remdesivir, a nucleotide analog, Arbidol (umifenovir), Prezcobix (darunavir, HIV-1 protease inhibitor/cobicistat, CYP3A inhibitor), Kaletra/Aluvia (lopinavir/ritonavir), an HIV-1 protease inhibitor, prophylactic antiviral CRISPR in human cells (PAC-MAN), GC376, AmnioBoost, concentrated allogeneic MSCs and cytokines derived from amniotic fluid, Astrostem-V, allogenic adipose-derived mesenchymal stem cells (HB-adMSCs), bone marrow-derived allogenic mesenchymal stem cells (BM-Allo-MSC), mesenchymal stem cells, allogenic adipose-derived mesenchymal stem cells (HB-adMSCs) haNK, natural killer cells, Ryoncil (remestemcel-L), allogenic mesenchymal stem cells, MultiStem, bone marrow stem cells, allogeneic T-cell therapies, Autologous Adipose-Tissue Derived Mesenchymal Stem Cells (ADMSCs) and allogeneic MSCs, CYNK-001, CAP-1002, allogenic cardiosphere-derived cells, PLX cell product, placenta-based cell therapy, Chimeric antigen receptors (CAR)/T cell receptors (TCR)-T cell therapy, natural killer cell-based therapy, small mobile stem (SMS) cells, IMS001, human embryonic stem cell-derived mesenchymal stem cells (hES-MSC), VIR-2703 (ALN-COV) siRNA, OT-101, a TGF-Beta antisense drug, inhaled mRNA, peptide conjugated antisense oligonucleotides, Ampligen, rintatolimod, BXT-25, glycoprotein, EDP1815, Ivermectin, tradipitant, a neurokinin-1 receptor antagonist, piclidenoson, A3 adenosine receptor agonist, Ryanodex (dantrolene sodium), a skeletal muscle relaxant, Jakafi/jakavi (ruxolitinib), nitazoxanide, antiprotozoal, peptides targeting the NP protein, interferon/peginterferon alpha-2b, PegIntron, Sylatron, IntronA, PegiHep, roscovitine seliciclib, cyclin-dependent kinase (CDK)2/9 inhibitor, ATYR1923, a fusion protein comprising immuno-modulatory domain of histidyl tRNA synthetase fused to the Fc region of a human antibody, a modulator of neuropilin-2, Leukine (sargramostim, rhu-Granulocyte macrophage colony stimulating factor), ADX-1612, HSP 90 inhibitor, DSTAT (dociparstat sodium), glycosaminoglycan derivative of heparin, BIO-11006, Recombinant human interferon alpha-1b, ST-001 nanoFenretinide (fenretinide), Activase (alteplase), tissue plasminogen activator (tPA), camostat mesylate, a transmembrane protease serine 2 (TMPRSS2) inhibitor, nitric oxide, Cozaar (losartan), an angiotensin II receptor blocker (ARB), Otezla (apremilast), an inhibitor of phosphodiesterase 4 (PDE4), IMU-838, a selective oral dihydroorotate dehydrogenase (DHODH) inhibitor, Colchicine, Brilacidin, a defensin mimetic, Metablok (LSALT peptide), a selective dipeptidase-1 antagonist, nafamostat, CD24Fc, an agent comprising nonpolymorphic regions of CD24 attached to the Fc region of human IgG1, Aplidin (plitidepsin), fadraciclib (CYC065), a cyclin-dependent kinase (CDK)2/9 inhibitor, Aviptadil, a synthetic form of Vasoactive Intestinal Polypeptide (RLF-100), solnatide, a synthetic molecule with a structure based on the lectin-like domain of human Tumour Necrosis Factor alpha, PP-001, MRx-4DP0004, a strain of Bifidobacterium breve isolated from the gut microbiome of a healthy human, ARMS-1, BLD-2660, a small molecule inhibitor of calpain (CAPN) 1, a small molecule inhibitor of CAPN2, a small molecule inhibitor of CAPN9, LAU-7b (fenretinide), N-803, an IL-15 “superagonist” (Nogapendekin alfa inbakicept), Rebif, interferon beta-1a, DIBI, an iron-binding polymer, EPAspire, an oral formulation of highly purified eicosapentaenoic acid free fatty acid (EPA-FFA) in gastro-resistant capsules, MN-166 (ibudilast), a small molecule macrophase migration inhibitory factor (MIF) inhibitor, a phosphodiesterase (PDE) 4 inhibitor, a PDE10 inhibitor, ADX-629, an orally available reactive aldehyde species (RASP) inhibitor, Calquence (acalabrutinib), a Bruton's tyrosine kinase (BTK) inhibitor, Auxora (CM4620-IE), a calcium release-activated calcium (CRAC) channel inhibitor Neumifil, a multivalent carbohydrate binding molecule, Diovan (valsartan), an angiotensin II receptor blocker (ARB), Yeliva (opaganib, ABC294640), an oral sphingosine kinase-2 (SK2) selective inhibitor, WP1122, a glucose decoy prodrug, Kineret (anakinra), an interleukin-1 receptor antagonist, a microbiome therapeutic, Coronzot, bemcentinib, a selective AXL kinase inhibitor, a synthesized nanoviricide drug, Chloroquine/Hydroxychloroquine, an antimalarial drug Senicapoc, vazegepant, a CGRP receptor antagonist, APN01, a recombinant soluble human Angiotensin Converting Enzyme 2, GP1681, a small molecule inhibitor of cytokine release, ST266, a cell-free biologic made from anti-inflammatory proteins secreted by placental cells, recombinant human plasma gelsolin (rhu-pGSN), pacritinib, an oral kinase inhibitor with specificity for JAK2, IRAK1 and CSFIR, Ruconest (recombinant human C1 esterase inhibitor), Cerocal (ifenprodil), NP-120, an NDMA receptor glutamate receptor antagonist targeting Glu2NB, Peginterferon lambda, Pepcid (famotidine), a histamine-2 (H2) receptor antagonist, heparin, a low molecular weight heparin (enoxaparin), an anticoagulant, Xeljanz (tofacitinib), a Janus kinase (JAK) inhibitor, Xpovio (selinexor), a selective inhibitor of nuclear export (SINE) compound, a pH barrier, transepithelial nebulized alkaline treatment, Luvox (fluvoxamine), a selective serotonin reuptake inhibitor, Micardis (telmisartan), brensocatib, a reversible inhibitor of dipeptidyl peptidase 1 (DPP1) Novaferon, RHB-107 (upamostat, WX-671), a serine protease inhibitor, UNI9011, FW-1022, DWRX2003, niclosamide, Lysteda/Cyklokapron/LB 1148 (tranexamic acid), an antifibrinolytic PUL-042 inhalation solution, ABX464, Gleevac (imatinib), Traumakine (interferon beta 1-a), Veyonda (idronoxil), Farxiga (dapagliflozin), a sodium-glucose cotransporter 2 (SGLTs) inhibitor, Gilenya (fingolimod), a sphingosine 1-phosphate receptor modulator, sPIF, a synthetic pre implantation factor, SNG001, an inhaled formulation of interferon beta-1a, Methylprednisolone, ciclesonide (Alvesco), hydrocortisone, corticosteroids Olumiant (baricitinib), a Janus kinase (JAK) inhibitor, dipyridamole (Persantine), an anticoagulant, AT-001, an aldose reductase inhibitor, Vascepa (icosapent ethyl), a form of eicosapentaenoic acid, OP-101, a dendrimer-based therapy, apabetalone (RVX-208), a selective BET (bromodomain and extra-terminal) inhibitor, Flarin (lipid ibuprofen), Almitrine, VP01, an Angiotensin II Type 2 receptor activator, leflunomide, a pyrimidine synthesis inhibitor, Pulmozyme (nebulised dornase alfa), a recombinant DNase enzyme, AQCH, MSTT1041A (anti-ST2, the receptor for IL-33), UTTR1147A (IL-22-Fc), CIGB-258, FSD-201, ultramicronized palmitoylethanolamide, PB1046, a long-acting sustained release human vasoactive intestinal peptide (VIP) analogue, PTC299, an oral small molecule inhibitor of dihydroorotate dehydrogenase (DHODH), raloxifene (Evista), an estrogen agonist/antagonist, losmapimod, an oral selective p38 mitogen activated protein kinase inhibitor, dutasteride, an anti-androgen, M5049, small molecule capable of blocking the activation of Toll-like receptor (TLR)7 and TLR8, Eritoran, a TLR-4 antagonist, desidustat, a hypoxia inducible factor prolyl hydroxylase inhibitor, merimepodib, an IMPDH inhibitor, azithromycin, Cenicriviroc, a chemokine receptor 2 and 5 dual antagonist, Firazyr (icatibant), a bradykinin B2 antagonist, Razoprotafib, Tie 2 activating compound (AKB-9778), or any combination thereof.

Antiviral agents provided include, but are not limited to abacavir; acemannan; acyclovir; acyclovir sodium; adefovir; alovudine; alvircept sudotox; amantadine hydrochloride; amprenavir; aranotin; arildone; atevirdine mesylate; avridine; cidofovir; cipamfylline; cytarabine hydrochloride; delavirdine mesylate; desciclovir; didanosine; disoxaril; edoxudine; efavirenz; enviradene; enviroxime; famciclovir; famotine hydrochloride; fiacitabine; fialuridine; fosarilate; trisodium phosphonoformate; fosfonet sodium; ganciclovir; ganciclovir sodium; idoxuridine; indinavir; kethoxal; lamivudine; lobucavir; memotine hydrochloride; methisazone; nelfinavir; nevirapine; palivizumab; penciclovir; pirodavir; ribavirin; rimantadine hydrochloride; ritonavir; saquinavir mesylate; somantadine hydrochloride; sorivudine; statolon; stavudine; tilorone hydrochloride; trifluridine; valacyclovir hydrochloride; vidarabine; vidarabine phosphate; vidarabine sodium phosphate; viroxime; zalcitabine; zidovudine; zinviroxime, interferon, cyclovir, alpha-interferon, and/or beta globulin. In certain aspects, other antibodies against viral proteins or cellular factors may be used in combination with a therapeutic composition described herein.

Antibacterial agents provided herein include, but are not limited to, β-lactam antibiotics, penicillins (such as natural penicillins, aminopenicillins, penicillinase-resistant penicillins, carboxy penicillins, ureido penicillins), cephalosporins (first generation, second generation, and third generation cephalosporins), and other β-lactams (such as imipenem, monobactams,), β-lactamase inhibitors, vancomycin, aminoglycosides and spectinomycin, tetracyclines, chloramphenicol, erythromycin, lincomycin, clindamycin, rifampin, metronidazole, polymyxins, sulfonamides and trimethoprim, and quinolines. Anti-bacterials also include, but are not limited to: Acedapsone, Acetosulfone Sodium, Alamecin, Alexidine, Amdinocillin, Amdinocillin Pivoxil, Amicycline, Amifloxacin, Amifloxacin Mesylate, Amikacin, Amikacin Sulfate, Aminosalicylic acid, Aminosalicylate sodium, Amoxicillin, Amphomycin, Ampicillin, Ampicillin Sodium, Apalcillin Sodium, Apramycin, Aspartocin, Astromicin Sulfate, Avilamycin, Avoparcin, Azithromycin, Azlocillin, Azlocillin Sodium, Bacampicillin Hydrochloride, Bacitracin, Bacitracin Methylene Disalicylate, Bacitracin Zinc, Bambermycins, Benzoylpas Calcium, Berythromycin, Betamicin Sulfate, Biapenem, Biniramycin, Biphenamine Hydrochloride, Bispyrithione Magsulfex, Butikacin, Butirosin Sulfate, Capreomycin Sulfate, Carbadox, Carbenicillin Disodium, Carbenicillin Indanyl Sodium, Carbenicillin Phenyl Sodium, Carbenicillin Potassium, Carumonam Sodium, Cefaclor, Cefadroxil, Cefamandole, Cefamandole Nafate, Cefamandole Sodium, Cefaparole, Cefatrizine, Cefazaflur Sodium, Cefazolin, Cefazolin Sodium, Cefbuperazone, Cefdinir, Cefepime, Cefepime Hydrochloride, Cefetecol, Cefixime, Cefinenoxime Hydrochloride, Cefinetazole, Cefinetazole Sodium, Cefonicid Monosodium, Cefonicid Sodium, Cefoperazone Sodium, Ceforanide, Cefotaxime Sodium, Cefotetan, Cefotetan Disodium, Cefotiam Hydrochloride, Cefoxitin, Cefoxitin Sodium, Cefpimizole, Cefpimizole Sodium, Cefpiramide, Cefpiramide Sodium, Cefpirome Sulfate, Cefpodoxime Proxetil, Cefprozil, Cefroxadine, Cefsulodin Sodium, Ceftazidime, Ceftibuten, Ceftizoxime Sodium, Ceftriaxone Sodium, Cefuroxime, Cefuroxime Axetil, Cefuroxime Pivoxetil, Cefuroxime Sodium, Cephacetrile Sodium, Cephalexin, Cephalexii Hydrochloride, Cephaloglycini, Cephaloridine, Cephalothin Sodium, Cephapirin Sodium, Cephradine, Cetocycline Hydrochloride, Cetophenicol, Chloramphenicol, Cliloramphenicol Palmitate, Chloramphenicol Pantotheniate Complex, Chloramphenicol Sodium Succinate, Chlorhexidine Phosphanilate, Chloroxylenol, Chlortetracycline Bisulfate, Chlortetracycline Hydrochloride, Cinoxacin, Ciprofloxacin, Ciprofloxacin Hydrochloride, Cirolemycin, Clarithromycin, Clinafloxacin Hydrochloride, Clildamycin, Clindamycin Hydrochloride, Clindamycin Palmitate Hydrochloride, Clindamycin Phosphate, Clofazimine, Cloxacillin Benzathine, Cloxacillin Sodium, Cloxyquin, Colistimethate Sodium, Colistin Sulfate, Coumermycin, Coumermycin Sodium, Cyclacillin, Cycloserine, Dalfopristin, Dapsone, Daptomycin, Demeclocycine, Demeclocycine Hydrochloride, Demecycline, Denofungin, Diaveridine, Dicloxacillin, Dicloxacillin Sodium, Dihydrostreptomycin Sulfate, Dipyrithione, Dirithromycin, Doxycycline, Doxycycline Calcium, Doxycycline Fosfatex, Doxycycline Hyclate, Droxacin Sodium, Enoxacin, Epicillin, Epitetracycline Hydrochloride, Erythromycin, Erythromycin Acistrate, Erythromycin Estolate, Erythromycin Ethylsuccinate, Erythromycin Gluceptate, Erythromycin Lactobionate, Erythromycin Propionate, Erythromycin Stearate, Ethambutol Hydrochloride, Ethionamide, Fleroxacin, Floxacillin, Fludalanine, Flumequine, Fosfomycin, Fosfomycin Tromethamine, Fumoxicillin, Furazolium Chloride, Furazolium Tartrate, Fusidate Sodium, Fusidic Acid, Gentamicin Sulfate, Gloximonam, Gramicidin, Haloprogin, Hetacillin, Hetacillin Potassium, Hexedine, Ibafloxacin, Imipenem, Isoconazole, Isepamicin, Isoniazid, Josamycin, Kanamycin Sulfate, Kitasamycin, Levofuraltadone, Levopropylcillin Potassium, Lexithromycin, Lincomycin, Lincomycin Hydrochloride, Lomefloxacin, Lomefloxacin Hydrochloride, Lomefloxacin Mesylate, Loracarbef, Mafenide, Meclocycline, Meclocycline Sulfosalicylate, Megalomicin Potassium Phosphate, Mequidox, Meropenem, Methacycline, Methacycline Hydrochloride, Methenamine, Methenamine Hippurate, Methenamine Mandelate, Methicillin Sodium, Metioprim, Metronidazole Hydrochloride, Metronidazole Phosphate, Mezlocillin, Mezlocillin Sodium, Minocycline, Minocycline Hydrochloride, Mirincamycin Hydrochloride, Monensin, Monensin Sodium, Nafcillin Sodium, Nalidixate Sodium, Nalidixic Acid, Natamycin, Nebramycin, Neomycin Palmitate, Neomycin Sulfate, Neomycin Undecylenate, Netilmicin Sulfate, Neutramycin, Nifuradene, Nifuraldezone, Nifuratel, Nifuratrone, Nifurdazil, Nifurimide, Nifuirpirinol, Nifurquinazol, Nifurthiazole, Nitrocycline, Nitrofurantoin, Nitromide, Norfloxacin, Novobiocin Sodium, Ofloxacin, Ormetoprim, Oxacillin Sodium, Oximonam, Oximonam Sodium, Oxolinic Acid, Oxytetracycline, Oxytetracycline Calcium, Oxytetracycline Hydrochloride, Paldimycin, Parachlorophenol, Paulomycin, Pefloxacin, Pefloxacin Mesylate, Penamecillin, Penicillin G Benzathine, Penicillin G Potassium, Penicillin G Procaine, Penicillin G Sodium, Penicillin V, Penicillin V Benzathine, Penicillin V Hydrabamine, Penicillin V Potassium, Pentizidone Sodium, Phenyl Aminosalicylate, Piperacillin Sodium, Pirbenicillin Sodium, Piridicillin Sodium, Pirlimycin Hydrochloride, Pivampicillin Hydrochloride, Pivampicillin Pamoate, Pivampicillin Probenate, Polymyxin B Sulfate, Porfiromycin, Propikacin, Pyrazinamide, Pyrithione Zinc, Quindecamine Acetate, Quinupristin, Racephenicol, Ramoplanin, Ranimycin, Relomycin, Repromicin, Rifabutin, Rifametane, Rifamexil, Rifamide, Rifampin, Rifapentine, Rifaximin, Rolitetracycline, Rolitetracycline Nitrate, Rosaramicin, Rosaramicin Butyrate, Rosaramicin Propionate, Rosaramicin Sodium Phosphate, Rosaramicin Stearate, Rosoxacin, Roxarsone, Roxithromycin, Sancycline, Sanfetrinem Sodium, Sarmoxicillin, Sarpicillin, Scopafungin, Sisomicin, Sisomicin Sulfate, Sparfloxacin, Spectinomycin Hydrochloride, Spiramycin, Stallimycin Hydrochloride, Steffimycin, Streptomycin Sulfate, Streptonicozid, Sulfabenz, Sulfabenzamide, Sulfacetamide, Sulfacetamide Sodium, Sulfacytine, Sulfadiazine, Sulfadiazine Sodium, Sulfadoxine, Sulfalene, Sulfamerazine, Sulfameter, Sulfamethazine, Sulfamethizole, Sulfamethoxazole, Sulfamonomethoxine, Sulfamoxole, Sulfanilate Zinc, Sulfanitran, Sulfas alazine, Sulfasomizole, Sulfathiazole, Sulfazamet, Sulfisoxazole, Sulfisoxazole Acetyl, Sulfisoxazole Diolamine, Sulfomyxin, Sulopenem, Sultamicillin, Suncillin Sodium, Talampicillin Hydrochloride, Teicoplanin, Temafloxacin Hydrochloride, Temocillin, Tetracycline, Tetracycline Hydrochloride, Tetracycline Phosphate Complex, Tetroxoprim, Thiamphenicol, Thiphencillin Potassium, Ticarcillin Cresyl Sodium, Ticarcillin Disodium, Ticarcillin Monosodium, Ticlatone, Tiodonium Chloride, Tobramycin, Tobramycin Sulfate, Tosufloxacin, Trimethoprim, Trimethoprim Sulfate, Trisulfapyrimidines, Troleandomycin, Trospectomycin Sulfate, Tyrothricin, Vancomycin, Vancomycin Hydrochloride, Virginiamycin, and/or Zorbamycin.

Anti-fungal agents provided herein include, but are not limited to, azoles, imidazoles, polyenes, posaconazole, fluconazole, itraconazole, amphotericin B, 5-fluorocytosine, miconazole, ketoconazole, Myambutol (Ethambutol Hydrochloride), Dapsone (4,4′-diaminodiphenylsulfone), Paser Granules (aminosalicylic acid granules), rifapentine, Pyrazinamide, Isoniazid, Rifadin IV, Rifampin, Pyrazinamide, Streptomycin Sulfate and Trecator-SC (Ethionamide) and/or voriconazole (Vfend™).

### EXAMPLES

Some aspects of the embodiments discussed above are disclosed in further detail in the following examples, which are not in any way intended to limit the scope of the present disclosure.

### Example 1

### SARS-CoV-2 Loss of Function Genetic Screens

To conduct global functional genetic screens for the identification of host factors that govern SARS-CoV-2 replication, an immunofluorescence (IF)-based assay previously optimized in drug screening was adapted (FIGS. 2A-2C) in several mammalian cell lines. For the screen, pooled-siRNAs targeting individual factors from the genome-wide Dharmacon ON-TARGET plus SMARTpool siRNA library were spotted into 384-well plates and reverse transfected into Caco-2 cells, a human epithelial cell line that can be easily transfected and supports efficient SARS-CoV-2 replication, using a previously described high-throughput methodology. An arrayed siRNA approach enables the identification of both early and late stage host proteins, including those that are essential or have moderate impact on replication, through monitoring the effects of genetic manipulation on multi-cycle replication in human cells in an arrayed (well-based) format. For the IF-based assay, infectivity is simply determined by the number of infected cells, those stained for the SARS-CoV-2 NP protein (FIG. 2B), over the total number of nuclei (DAPI) within each well. Uninfected cells, as well as a non-targeting scrambled siRNA and an siRNA targeting ACE2, were included as negative and positive controls, respectively, on each plate. The assay was run in duplicate and yielded an r=0.66. As shown in FIG. 2C, the primary whole genome siRNA screen yielded 264 potential factors (Z<−2) on which the host depends in order to replicate, including TMPRSS2 the serine protease that primes the SARS-CoV-2 Spike protein for viral entry. 81 potential factors (Z>1.8) that limit SARS-CoV-2 infection were also identified. Hits identified in the primary screen were subjected to a subsequent round of siRNA screening utilizing the same individual pooled-siRNAs. Following this round of screening, 190 cellular factors were validated as proviral factors that are necessary for SARS-CoV-2 replication and 21 factors were identified as antiviral, they limit SARS-CoV-2 replication. A final validation of these data was performed by subjecting hits from the validation screen to a subsequent round of siRNA screening now using individual rather than pooled siRNAs. This final round of validation screening yielded 125 proviral factors that validated with 2 or more individual siRNAs.

### Example 2

### ANN Predictions of Drug-Protein Interactions

Of the 345 proviral and antiviral targets identified from the genome-wide siRNA screen, a training set curation campaign was initiated for each. Repositories such as PubChem contain small molecule screening data for many targets, including the ones identified in this study, and these data points serve as inputs to train ligand-based predictive models. A sufficiently-sized training set is required for any probabilistic model to ensure accuracy of predictions when challenged with cross-validation testing. A minimum of 100 molecules were determined to be a sufficient number of measurements for both Inactive (true negative) as well as Active (true positive) data points from high quality confirmatory binding assays. Manual checks were required with many targets, as the deposited bioassay title or descriptions were not always accurately reflected in PubChem. This diligence and curation step led to eight target proteins and their respective molecule training datasets. Deep learning predictive models were developed, configured, and optimized as binary classifiers for each of the eight protein targets. These models were then applied to a prefiltered repurposable drug list and used to make predictions as to Active or Inactive for each, along with individual probability scores.

These compounds were next filtered based upon novelty, non-obviousness, regulatory stage, and physical availability. To be deemed as novel, the compound must not be associated or mentioned with the target gene, gene product, or gene isoforms. To be deemed as nonobvious, the compound must not have known antiviral activity either in vitro or in vivo. Computational discoveries, docking or image-based screens were not considered for the nonobvious criteria. Searches were conducted using public databases such as PubChem, DrugBank, ChEMBL, PubMed, Google, and Google Patents. The compounds that were considered novel and nonobvious and had patent freedom to operate were then assessed for their regulatory development. Those that had at minimum successfully completed a Phase 1 clinical trial and had not been subsequently terminated due to toxicity/safety issues were selected. Searches for regulatory criteria were conducted using clinicaltrials.gov, International Clinical Trials Registry Platform (ICTRP), and Google. Lastly, compounds were filtered to a list that have accessibility through either US vendors or Sanford Burnham Prebys Medical Discovery Institute's compound library. This triage funnel led to 25 probability-ranked hits from each of the eight target-model runs, resulting in roughly 200 compounds for experimental validation.

### Example 3

### Proof-of-Concept-Testing

The fact that Baculoviral IAP repeat-containing protein 2 (BIRC2), also known as cellular inhibitor of apoptosis 1 (cIAP1), was amongst the list of targets for which drug-protein interactions were predicted, presented a unique opportunity to conduct a proof-of-concept test of the workflow. Smac mimetics, a class of small-molecule peptidomimetics derived from a conserved binding motif of Smac (second mitochondria-derived activator of caspases), are an endogenous protein inhibitor of IAPs. It had been previously reported that Smac mimetic compounds that can target the inhibitor of apoptosis protein cIAP1 (Birc2) harbor HIV latency reactivation activity. Neither the bivalent next generation Smac mimetic compound, Ciapavir (SBI-0953294), nor the Astra Zeneca compound 5582 (AZD5582) which is also used for comparative studies for HIV-1 reactivation, were included in the training dataset described herein. The algorithm predicted high confidence binding of both to Birc2. The antiviral activity of Ciapavir and AZD5582 were both tested within the cellular antiviral assay described above, but now within HEK293T cells stably expressing ACE2 (FIGS. 3A-B). Both compounds displayed excellent activity with no observable cytotoxicity up to 10 mM compound; Caipavir exhibited an IC₅₀=0.26 mM.

### Example 4

### Example Repurposable Drug

Clidinium was in the list of 25 compounds predicted with a high confidence to bind BIRC2. Clidinium is a synthetic anticholinergic agent which targets Muscarinic acetylcholine receptor M3 (antagonist) and has been shown in experimental and clinical studies to have a pronounced antispasmodic and antisecretory effect on the gastrointestinal tract. Clidinium is approved in combination with chlordiazepoxide hydrochloride (Librax, 1966). Librax is indicated to control emotional and somatic factors in gastrointestinal disorders. Librax may also be used as adjunctive therapy in the treatment of peptic ulcer and in the treatment of the irritable bowel syndrome (irritable colon, spastic colon, mucous colitis) and acute enterocolitis. Clidinium use against SARS-CoV-2 infection has never been previously reported or anticipated, and represents an example of novel, nonobvious, and unexpected potential as a COVID therapeutic. In addition to Clidinium, roughly 199 additional compounds that target the genes of interest (GOI) that were found to be involved in SARS-CoV-2 pathogenesis were identified.

### Example 5

### In Vitro and Ex Vivo Validation of Drug Candidates with SARS-CoV-2 Antiviral Activity

The roughly 200 repurposable drugs predicted to target one or more of eight different host factors and thereby to potentially possess anti-SARS-CoV-2 activity will enter into the validation funnel shown in FIG. 1. Compounds will first be tested within an in vitro cellular model of infection in Caco-2 cells (see FIGS. 2A-2C). Caco-2 cells are immortalized cells of the human epithelium, which represents the initial site of viral infection. These cells can be easily grown and support efficient SARS-CoV-2 replication, thus maximizing the number of compounds that can be tested. Both infection and cytotoxicity will be monitored in order to identify the drugs that display the greatest therapeutic window as measured by the selectivity index (SI=CC₅₀/IC₅₀). From these roughly 200 repurposed drugs, those displaying the greatest SI values and chosen to maximize the predicted host target diversity, will then be prioritized for validation within primary iPSC-derived pneumocytes. These surface epithelial cells of the alveoli, represent a biologically relevant model system.

### Example 6

### Dose Response Studies for IC₅₀, CC₅₀, and SI Calculations

The roughly 200 compounds predicted to target host factors that impact on SARS-CoV-2 replication will be tested in 8-point dose response infectivity and cytotoxicity assays to determine IC₅₀, CC₅₀, and selectivity index (SI) values. Compounds will be pre-spotted and Caco-2 cells added to bring the highest compound concentration to 10 μM. The assay and data analysis to determine infectivity and viability/cytotoxicity will be performed as described above for the immunofluorescence-based genetic screen. Remdesivir will be similarly spotted on each experimental plate in a dose response to bench mark results against the current COVID-19 standard of care. Based on all infectivity and cytotoxicity values, a 4-parameter logistic non-linear regression model will be used to calculate IC₅₀ and CC₅₀ concentration values. These assays will enable establishment of SI values, representing a measure of a drug's inhibitory potency with respect to its cytotoxicity, for each compound. Compounds displaying a clear dose response and the greatest SI values (SI>10, and IC₅₀<1 μM), representing a diverse group of predicted host protein targets, will be prioritized.

### Example 7

### Validation within iPSC-Derived Pneumocytes

iPSC-derived pneumocytes will be derived from human embryonic stem cell lines hPSCl (H9,WiCell) and induced to differentiate to type 2 pneumocytes using a two-step differentiation protocol (1st to endoderm followed by a modified alveolar differentiation protocol) which has been previously published. Eleven days following the 2^{nd} differentiation step, the type 2 pneumocyte phenotype will be validated by flow cytometry staining for surfactant protein C, mucin-1, surfactant protein B, and the epithelial surface marker CD54. The prioritized drugs will then be added to pneumocytes for a 5 μM final concentration and will be incubated for 2 h before being challenged with SARS-CoV-2 (MOI of 0.0625). 48 h post infection cells will be fixed, permeabilized, and stained for analysis of infection. Infection data will be analyzed as described above (FIGS. 2A-C).

### Example 8

### In Vivo Validation of Drug Candidates' Potential for Use as a COVID-19 Therapeutic

Repurposed drugs displaying anti-SARS-CoV-2 within in vitro and ex vivo human cellular models of infection, will be prioritized for follow up for in vivo validation within animal models of infection, including hamsters, hACE2 transgenic mice, and hACE2-Adenovirus sensitized mice. To identify compounds with the best potential for the treatment of COVID-19 two mouse models in a stepwise fashion will be used. In the first step the hACE2-Adenovirus model will be used for initial prophylactic studies designed to quickly identify drugs that show the greatest prophylactic antiviral activity. In this model, cells in the lung transduced by the adenovirus vector become susceptible for SARS-CoV-2 infection through expression of the required hACE2 receptor. After intranasal challenge with SARS-CoV-2, the virus replicates to titers around 10⁵ pfu in the lungs of these animals by day 3, but the animals do not exhibit signs of disease. Comparison of virus titers in mice receiving the hACE2-adenovirus vector and drug treatment compared to vehicle control will quickly identify compounds that exhibit antiviral activity in vivo. In a second step the transgenic human ACE2 mouse will be used to further characterize the best compounds displaying antiviral activity in vivo in the first mouse model for their prophylactic and therapeutic potential in reducing viral titers and disease. In this infection model body weight loss, lung pathology, and viral titers will be tracked. This in contrast to the hACE2 Adenovirus sensitized mice, the hACE2 transgenic mice display viral titers in lungs that are two logs higher, the virus also replicates in other organs, such as brain, and the infection is lethal and characterized by ARDS like symptoms, which will allow for the acquisition of cytokine levels and to assess histopathology.

### Example 9

### Efficacy Testing in Mouse Embryonic Fibroblasts

Before advancing anti-COVID-19 compounds into in vivo studies, antiviral assays will be conducted with mouse embryonic fibroblast derived from the transgenic human ACE2 mouse. The prioritized compounds, displaying the greatest SI in human cell lines and validated antiviral activity within iPSC derived pneumocytes, will be confirmed in 5-point dose response infectivity and cytotoxicity assays to determine IC₅₀, CC₅₀, and selectivity index (SI) values in these MEFs. The assay and data analysis to determine infectivity and viability/cytotoxicity will be performed as described for the immunofluorescence-based screen.

Given that the prioritized compounds tested in MEFs consist of known drugs or advanced compounds with optimized pharmacokinetics and safety, the compounds displaying the greatest SI in mice and targeting as many different host factors as possible will be advanced to in vivo testing.

### Example 10

### In Vivo Prophylactic Proof-of-Concept for Anti-COVID-19 Activity

These prioritized compounds will be tested in prophylaxis studies, using the maximum tolerable dose (MTD) that achieves concentrations higher than the IC₉₀ in target tissues (lungs) for virus replication using IP dosing, for in vivo efficacy in the mouse model based on adeno-associated virus (AAV)-mediated expression of hACE2 (hACE2-AAV) delivered to the respiratory tract.

In these experiments viral seed stocks for non-replicating E1/E3 deleted adenoviral vectors based on human adenovirus type-5 (HAdV-C5, referred to as Ad throughout) without an antigen (Ad-Empty), or expressing the human angiotensin-converting enzyme 2 (Ad-hACE2) receptor under the control of a CMV promoter can be used. Adenovirus vectors will be obtained from the Iowa Viral Vector Core Facility. Viral stocks can be amplified to high titers following infection of the complementing T-Rex™-293 cells and purification using two sequential rounds of cesium chloride (CsCl) ultracentrifugation, as described previously. Infectious titer will be determined using a tissue culture infectious dose-50 (TCID₅₀) end-point dilution assay, and physical particle titer will be quantified by micro-bicinchoninic acid (microBCA) protein assay, both described previously. It should be noted that the Ad5 vectors can be propagated in complement 293 cells, but they are replication incompetent due to the E1/E3 deletion. In non-complementing cells, the Ad5 virus enters, expresses the transgene, but does not propagate.

For challenge experiments SARS-CoV-2, isolate USA-WA1/2020 (BEI resources; NR-52281) under BSL-3 containment in accordance to the biosafety protocols developed by the Icahn School of Medicine at Mount Sinai will be used. Viral stocks will be grown in Vero-E6 cells for 72 hrs and validated by genome sequencing.

Mice will be housed in a BSL2 facility at ISMMS and transferred to the ISMMS BSL3 when ready to be infected. 2.5×10⁸PFU of Ad5 expressing hACE2 will be delivered intranasally into 6-week-old Balb/c mice. At day 5 post-transduction, lungs of the transduced animal will be harvested, and expression of hACE2 will be monitored by Western blot. After validation of ACE2 expression within mouse lungs, then groups of transduced mice (six per compound, three males and three females) will be inoculated for each condition and equal numbers of controls will be included (mock treated, challenged mice; and treated, mock-challenged mice). Dosing will be selected according to PK data achieving concentrations higher than the IC₉₀ in the lungs of treated mice. As positive controls, Remdesivir will be used at 50 mg/kg twice a day (this results in approximately two logs reduction in viral replication in the lungs,). Mice will be intranasally infected with 10⁵ pfu of human COVID-19). At day three post infection mice will be sacrificed, lungs will be extracted, homogenized and virus titers present in these homogenates will be determined. Untreated challenged mice can show high viral titers by day 3 (around 10⁵ pfu per lung) and compounds with antiviral activity should reduce titers. The compounds displaying the greatest reduction in viral titers will be subsequently characterized within additional prophylactic and therapeutic in vivo studies using the more expensive hACE2 transgenic mouse model, that in addition to higher viral replication in lungs, results in ARDS and lethality, as well as in extrapulmonary replication.

### Example 11

### In Vivo Prophylactic and Therapeutic Characterization of Anti-COVID-19 Activity

For these prioritized compounds, the MTD achieving concentrations higher than the IC₉₀ in target tissues (lungs) for virus replication using IP dosing will be tested. In these studies the transgenic human ACE2 mouse infected with SARS-CoV-2 will be utilized.

For challenge experiments SARS-CoV-2, isolate USA-WA1/2020 (BEI resources; NR-52281) under BSL-3 containment in accordance to the biosafety protocols developed by the Icahn School of Medicine at Mount Sinai will be used. Viral stocks will be grown in Vero-E6 cells for 72 hrs and validated by genome sequencing.

Mice will be housed in a BSL2 facility at ISMMS and transferred to the ISMMS BSL3 when ready to be infected. Groups of 16 5-6-week ACE2-C57BL/6 mice (equal representation of male and female mice) will be inoculated for each condition and equal numbers of controls will be included (mock treated, challenged mice; and treated, mock-challenged mice). Remdesivir will again be used as a positive control. Mice can be intranasally infected with five LD₅₀s of human COVID-19. Mice will be followed for body weight loss and survival. Four mice per treatment condition will be sacrificed at days 3 and 6, lungs will be extracted, one lung will be homogenized and virus titers and proinflammatory cytokines present in these homogenates will be determined. The second lung will be used for histopathology. Untreated challenged mice can start losing weight at day 2-3 postinfection and by day 7-8, they will have reached 25% bodyweight loss at which point they are humanely euthanized. For those compounds in which prophylactic treatment also works in this mouse model, therapeutic treatments can be conducted in which compounds are dosed 12 hours, 24 hours and 36 hours after infection.

### Example 12

### Selected Compound Testing

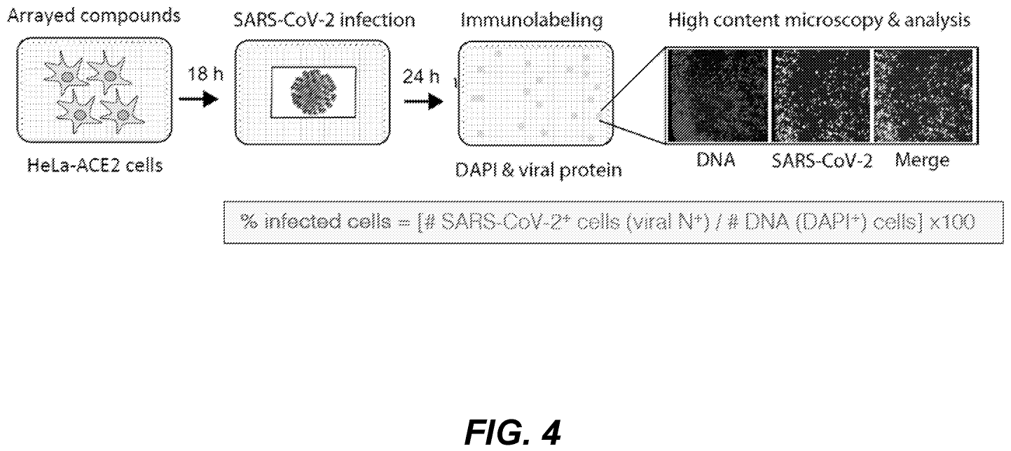

This example describes the experimental testing of selected drugs predicted to target one or more of eight different host factors and thereby to potentially possess anti-SARS-CoV-2 activity. FIG. 4 provides a non-limiting exemplary compound screening workflow employed herein. First, metabolic assays (Cell Titer Glow, Promega) were conducted to determine cytotoxicity of selected compounds at 1 and 5 μM. Second, the effect of selected compounds on viral replication at 1 and 5 μM was evaluated (with DAPI/DNA measurements also performed in parallel and providing Cell Number in the well to evaluate cell viability).

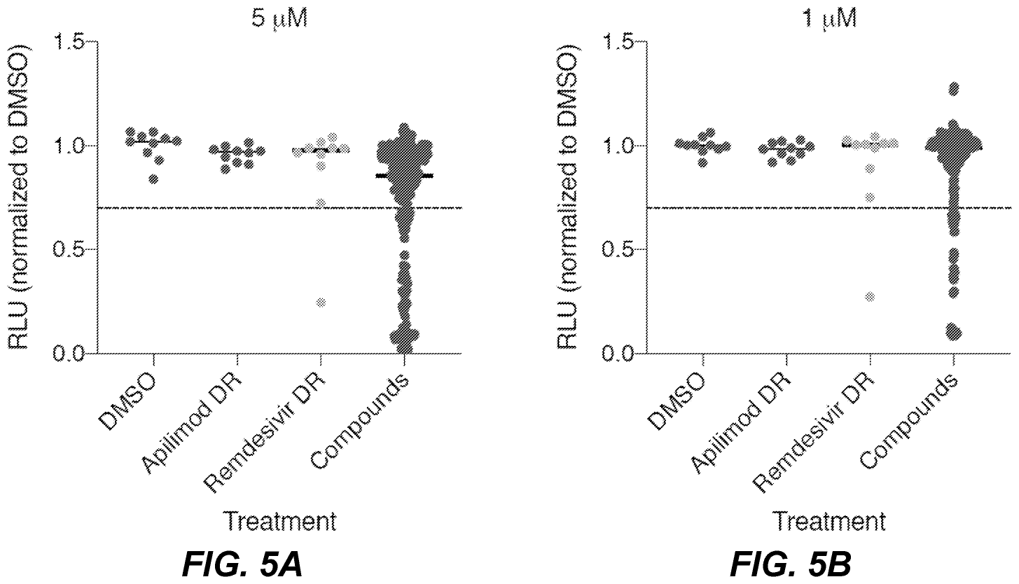

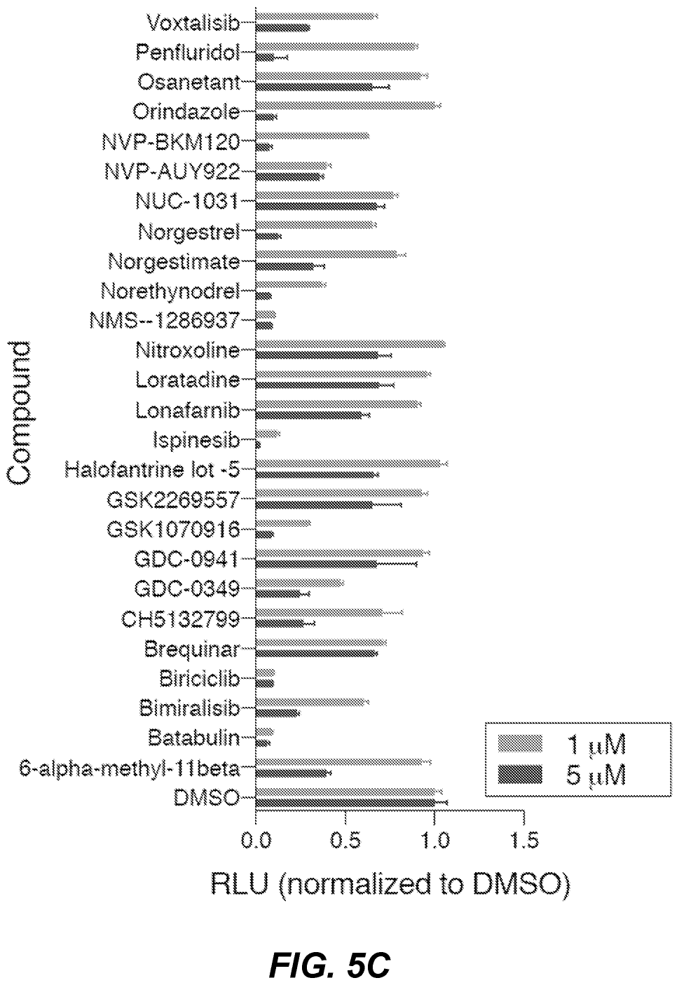

FIGS. 5A-5C depict cytotoxicity data. FIG. 5A-FIG. 5B depict the cytotoxicity of controls (DMSO, Apilimod DR, Remdesivir DR) and selected compounds when tested at 5 μM (FIG. 5A) and 1 μM (FIG. 5B). If considering compounds with a RLU<0.7 are cytotoxic, then 26 selected compounds are cytotoxic at 5 uM with this threshold and 13 selected compounds are cytotoxic at 1 uM with this threshold. FIG. 5C depicts data related to cytotoxicity of the indicated compounds at 5 μM and 1 μM.

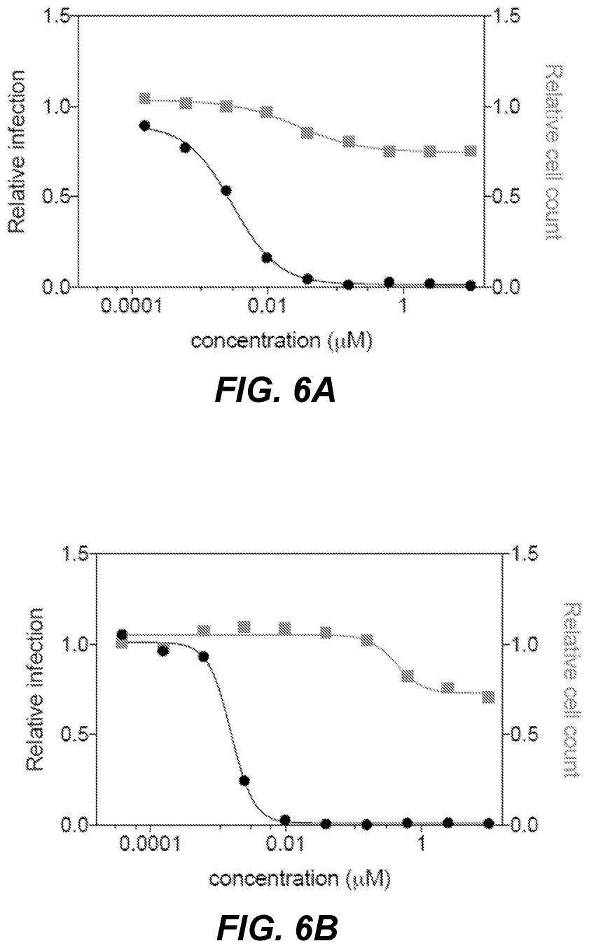

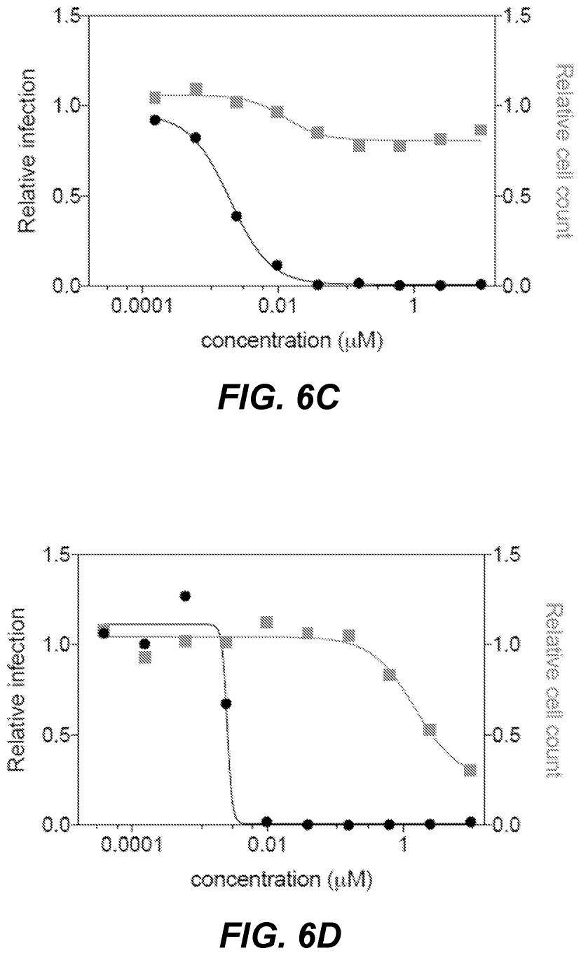

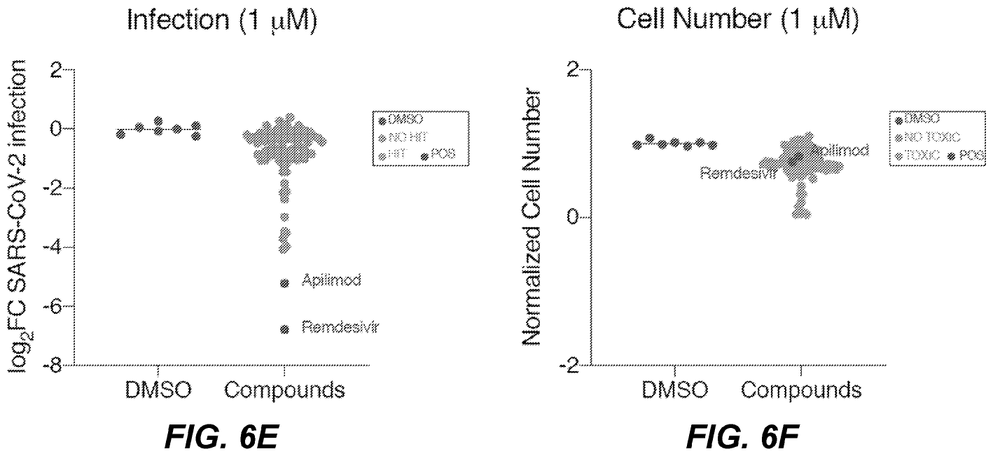

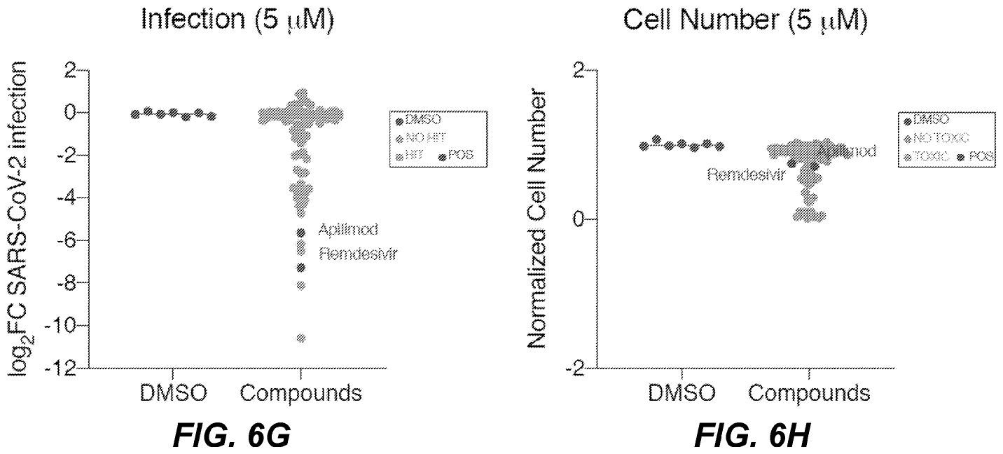

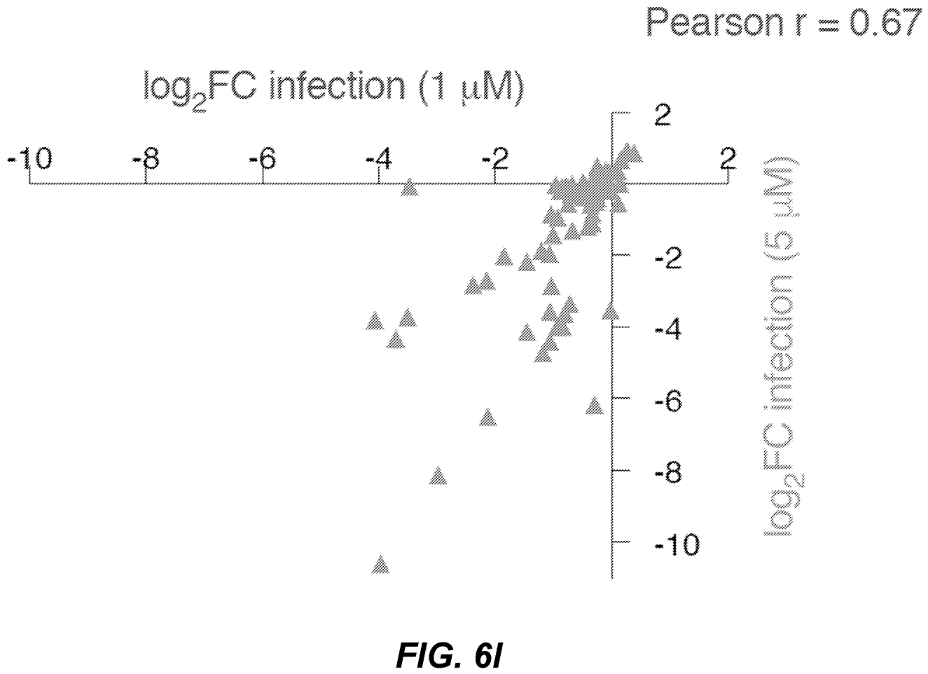

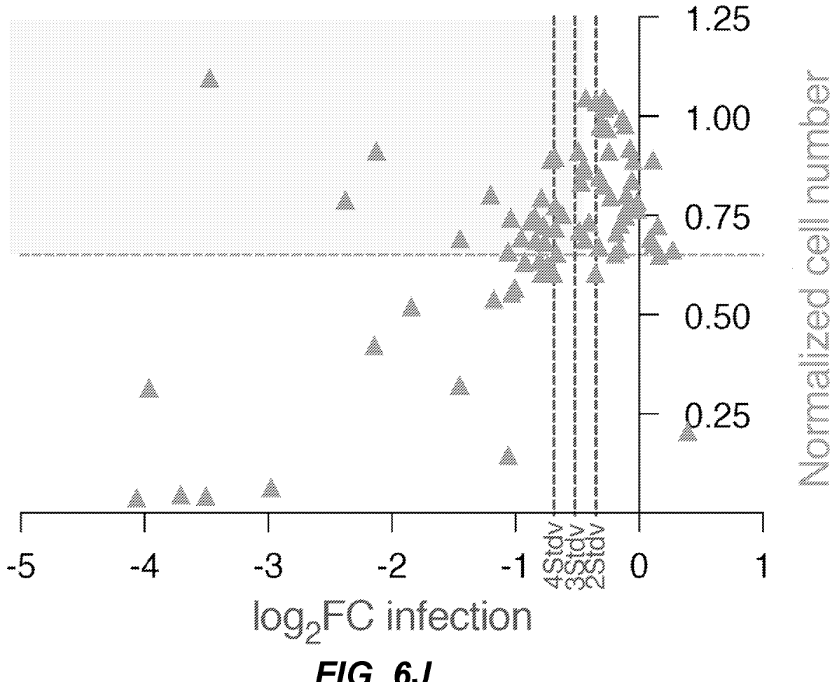

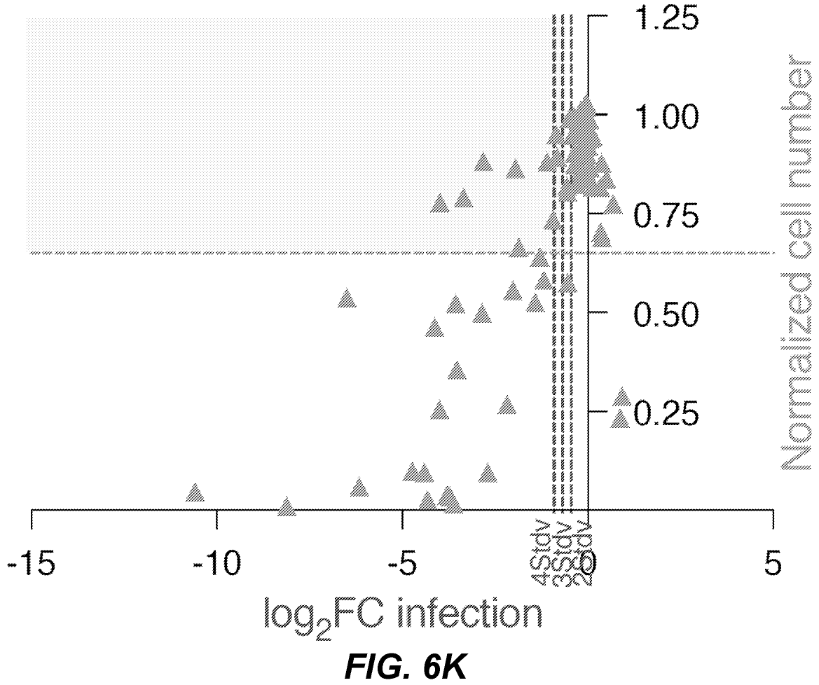

FIGS. 6A-6F depict infection data. FIGS. 6A-6D depict data related to the relative infection (circles) and relative cell count (squares) of the Apilimod (FIG. 6A, FIG. 6C) and Remdesivir (FIG. 6B, FIG. 6D) controls tested in the 1 μM screen (FIGS. 6A-6B) and the 5 μM screen (FIGS. 6C-6D) described below. FIGS. 6E-6H depict the effect of selected compounds on log₂FC SARS-CoV-2 infection (FIG. 6E and FIG. 6G) and normalized cell number (FIG. 6F and FIG. 6H) at 1 μM (FIGS. 6E-6F) and at 5 μM (FIGS. 6G-6H).

The effect of each selected compound on SARS-CoV-2 replication and cell viability was tested in triplicate (n=3) at 1 μM (FIGS. 6E-6F). Each dot depicted in FIG. 6E represents the average log₂FC infection of each compound calculated relative to the negative control DMSO. Each dot depicted in FIG. 6F represents the average normalized cell number of each compound calculated relative to the negative control DMSO. Apilimod and Remdesivir were tested at 0.625 μM (FIGS. 6E-6F).

The effect of each selected compound on SARS-CoV-2 replication and cell viability was tested in triplicate (n=3) at 5 μM (FIGS. 6G-6H). Each dot depicted in FIG. 6G represents the average log₂FC infection of each compound calculated relative to the negative control DMSO. Each dot depicted in FIG. 6H represents the average normalized cell number of each compound calculated relative to the negative control DMSO. Apilimod and Remdesivir were tested at 2.5 μM (FIGS. 6G-6H).

FIG. 6I depicts correlation analysis of the effect of the selected compounds on log₂FC SARS-CoV-2 infection at 1 μM and at 5 μM. The effect of each compound on SARS-CoV-2 replication was tested in triplicate (n=3) both at 1 and 5 uM. Each triangle represents the average log₂FC infection of each compound. The log₂FC infection was calculated relative to the negative control DMSO.

Correlation of log₂FC infection with cell number was investigated. FIGS. 6J-6K depict correlation analysis of the effect of the selected compounds on normalized cell number and log₂FC SARS-CoV-2 infection at 1 μM (FIG. 6J) and at 5 μM (FIG. 6K). Dotted lines represent threshold for considering compounds antiviral (log₂FC<4/3/2*Stdv of negative control DMSO, in blue), and non-toxic (Normalized Cell number>0.65, in pink). Each triangle (in grey) represents the average log₂FC (n=3) and normalized cell number of each compound relative to DMSO.

With threshold of infection log₂FC<2*Stdv of negative control DMSO, and normalized cell number>0.65, there are 35 small molecules that show antiviral activities (43% of compounds tested). The data generated related to these small molecules are shown below in Tables 9A-9C. Tables 2-8 above list these verified compounds with respect to the putative host proteins targeted. These identified compounds will be tested in dose-response infectivity and cytotoxicity assays.

TABLE 9A
Selected Compound Testing Results

| | % | Normalized | log2FC | Average | % | Normalized | log2FC | Average |
|---|---|---|---|---|---|---|---|---|
| | Infection | Infection | Infection | Infection | Infection | Infection | Infection | Infection |
| Treatment | (1 uM) | (1 uM) | (1 uM) | (1 uM) | (5 uM) | (5 uM) | (5 uM) | (5 uM) |
| ADX-47273 | 21.76934 | 0.82877137 | −0.2709539 | −0.4314888 | 14.05526 | 0.91281923 | −0.1315989 | −0.3578565 |
| ADX-47273 | 18.58025 | 0.70736087 | −0.4994817 | | 10.48518 | 0.68096029 | −0.5543574 | |
| ADX-47273 | 18.26676 | 0.69542612 | −0.5240308 | | 11.76985 | 0.76439321 | −0.3876131 | |
| Ampicillin | 12.65471 | 0.48177213 | −1.0535771 | −0.9564312 | 15.72706 | 1.02139432 | 0.03053994 | −0.0333969 |
| Ampicillin | 14.89758 | 0.56715949 | −0.8181736 | | 15.05413 | 0.97769086 | −0.0325497 | |
| Ampicillin | 13.15589 | 0.50085235 | −0.9975427 | | 14.38463 | 0.93421017 | −0.0981809 | |
| Bimiralisib | 10.02865 | 0.38179651 | −1.3891242 | −1.4499272 | 0.78712 | 0.05111953 | −4.2899817 | −4.1233107 |
| Bimiralisib | 13.46198 | 0.51250537 | −0.964361 | | 0.89574 | 0.05817386 | −4.1034852 | |
| Bimiralisib | 6.58363 | 0.2506426 | −1.9962964 | | 0.97818 | 0.06352793 | −3.9764653 | |
| CC-401 | 22.6391 | 0.86188364 | −0.214435 | −0.3224153 | 8.99259 | 0.58402399 | −0.7759005 | −0.8279807 |
| CC-401 | 20.63554 | 0.78560695 | −0.3481204 | | 8.27136 | 0.53718369 | −0.8965126 | |
| CC-401 | 19.84205 | 0.75539833 | −0.4046905 | | 8.77323 | 0.56977765 | −0.8115291 | |
| Cefonicid | 19.06724 | 0.72590086 | −0.4621556 | −0.4928567 | 16.96938 | 1.10207683 | 0.1402248 | 0.04522801 |
| Cefonicid | 19.18681 | 0.73045296 | −0.4531367 | | 13.66546 | 0.88750365 | −0.172175 | |
| Cefonicid | 17.77653 | 0.67676278 | −0.5632779 | | 17.29486 | 1.12321513 | 0.16763428 | |
| CH5132799 | 11.04728 | 0.42057634 | −1.2495604 | −1.0588587 | 1.27348 | 0.08270619 | −3.5958609 | −3.5650778 |
| CH5132799 | 14.02728 | 0.53402667 | −0.9050163 | | 1.24658 | 0.08095917 | −3.6266617 | |
| CH5132799 | 12.93475 | 0.49243342 | −1.0219994 | | 1.38696 | 0.09007615 | −3.472711 | |
| Cintirorgon | 14.12762 | 0.53784667 | −0.8947332 | −0.8422556 | 18.25585 | 1.18562665 | 0.24564978 | −0.1089951 |
| (Cinitirogon) | | | | | | | | |
| Cintirorgon | 15.44393 | 0.58795935 | −0.7662117 | | 12.75999 | 0.82869788 | −0.2710819 | |
| Cintirorgon | 14.41359 | 0.54873371 | −0.8658219 | | 12.49331 | 0.81137834 | −0.3015533 | |
| Clidinium | 19.06484 | 0.72580949 | −0.4623372 | −0.7650697 | 12.64633 | 0.82131623 | −0.2839903 | −0.1068823 |
| Clidinium | 14.63816 | 0.55728322 | −0.8435174 | | 14.20826 | 0.92275581 | −0.1159792 | |
| Clidinium | 13.23077 | 0.50370307 | −0.9893546 | | 16.26794 | 1.05652179 | 0.07932252 | |
| DADLE | 17.22456 | 0.65574897 | −0.6087845 | −0.6223235 | 13.21983 | 0.8585622 | −0.2200054 | −0.2501128 |
| DADLE | 16.52485 | 0.6291106 | −0.6686144 | | 10.80508 | 0.7017362 | −0.5109993 | |
| DADLE | 17.45548 | 0.66454023 | −0.5895716 | | 15.19267 | 0.98668835 | −0.0193336 | |
| Daprodustat | 15.16775 | 0.57744502 | −0.7922445 | −0.852599 | 13.9839 | 0.90818475 | −0.1389423 | −0.0909252 |
| Daprodustat | 16.56845 | 0.63077048 | −0.664813 | | 13.08411 | 0.84974786 | −0.2348933 | |
| Daprodustat | 12.24771 | 0.46627741 | −1.1007396 | | 16.51491 | 1.07256126 | 0.10106005 | |
| Desogestrel | 21.24342 | 0.80874929 | −0.3062355 | −0.4489167 | 13.40224 | 0.87040883 | −0.2002349 | −0.1002295 |
| Desogestrel | 20.95165 | 0.79764144 | −0.3261877 | | 15.07555 | 0.97908199 | −0.0304984 | |
| Desogestrel | 16.00946 | 0.60948941 | −0.7143269 | | 14.66883 | 0.95266755 | −0.0699553 | |
| Etonogestrel | 18.91892 | 0.72025423 | −0.4734219 | −0.4064805 | 13.41762 | 0.87140768 | −0.1985803 | −1.1873297 |
| Etonogestrel | 21.80537 | 0.83014306 | −0.2685681 | | 13.07339 | 0.84905165 | −0.2360758 | |
| Etonogestrel | 18.86615 | 0.71824525 | −0.4774516 | | 1.76211 | 0.11444028 | −3.1273332 | |
| GDC-0941 | 3.8228 | 0.14553621 | −2.78055 | −2.373968 | 5.4663 | 0.355009 | −1.4940725 | −2.8203018 |
| GDC-0941 | 5.66198 | 0.21555486 | −2.213873 | | 0.27366 | 0.01777286 | −5.8141807 | |
| GDC-0941 | 6.01139 | 0.2288571 | −2.1274811 | | 6.92582 | 0.44979756 | −1.1526523 | |
| GSK2269557 | 17.26351 | 0.65723182 | −0.6055258 | −0.7144268 | 1.29159 | 0.08388235 | −3.575489 | −3.346724 |
| GSK2269557 | 15.32215 | 0.58332312 | −0.7776328 | | 0.46985 | 0.03051442 | −5.034365 | |
| GSK2269557 | 15.50926 | 0.5904465 | −0.7601217 | | 5.71328 | 0.37104912 | −1.4303179 | |
| Halofantrine | 12.77766 | 0.48645291 | −1.0396279 | −1.2018123 | 5.04202 | 0.32745412 | −1.6106353 | −1.864201 |
| lot −4 | | | | | | | | |
| Halofantrine | 11.03281 | 0.42002546 | −1.2514513 | | 3.60531 | 0.23414695 | −2.0945138 | |
| lot −4 | | | | | | | | |
| Halofantrine | 10.56208 | 0.4021045 | −1.3143576 | | 4.16173 | 0.27028366 | −1.8874538 | |
| lot −4 | | | | | | | | |
| Halofantrine | 12.91611 | 0.49172378 | −1.02408 | −1.062433 | 4.52138 | 0.29364114 | −1.767874 | −1.9470474 |
| lot −5 | | | | | | | | |
| Halofantrine | 13.03677 | 0.49631738 | −1.0106651 | | 4.03641 | 0.26214475 | −1.9315644 | |
| lot −5 | | | | | | | | |
| Halofantrine | 11.81564 | 0.44982825 | −1.1525538 | | 3.48929 | 0.22661203 | −2.1417036 | |
| lot −5 | | | | | | | | |
| Ivacaftor | 15.71046 | 0.5981063 | −0.7415262 | −0.9235492 | 9.38053 | 0.60921876 | −0.7149677 | −0.9409006 |
| Ivacaftor | 12.72529 | 0.48445915 | −1.0455531 | | 7.91146 | 0.51380998 | −0.9606932 | |
| Ivacaftor | 13.28394 | 0.50572728 | −0.9835685 | | 6.95281 | 0.45155043 | −1.147041 | |
| Licofelone | 15.52745 | 0.59113901 | −0.7584307 | −0.7410808 | 7.45342 | 0.48406256 | −1.0467346 | −0.5549704 |
| Licofelone | 15.00975 | 0.57142987 | −0.8073516 | | 13.23468 | 0.85952664 | −0.2183857 | |
| Licofelone | 16.65311 | 0.63399354 | −0.65746 | | 11.67092 | 0.7579682 | −0.3997908 | |
| Lonafarnib | 17.44278 | 0.66405673 | −0.5906216 | −0.6666504 | 6.30411 | 0.40942059 | −1.2883444 | −1.3000328 |
| Lonafarnib | 16.89587 | 0.64323555 | −0.636581 | | 5.67156 | 0.36833961 | −1.4408915 | |
| Lonafarnib | 15.37411 | 0.58530126 | −0.7727487 | | 6.83895 | 0.44415579 | −1.1708623 | |
| Loratadine | 21.47702 | 0.81764258 | −0.2904578 | −0.3262626 | 8.31414 | 0.53996204 | −0.8890701 | −1.089355 |
| Loratadine | 19.44591 | 0.74031705 | −0.4337848 | | 6.30048 | 0.40918484 | −1.2891754 | |
| Loratadine | 22.01835 | 0.83825133 | −0.2545452 | | 7.23412 | 0.46982011 | −1.0898196 | |
| LXS-196 | 3.521 | 0.13404651 | −2.8991945 | −2.1233729 | 0.35165 | 0.02283792 | −5.452425 | −6.4970966 |
| (lxs196) | | | | | | | | |
| LXS-196 | 8.1351 | 0.30970796 | −1.6910196 | | 0.0917 | 0.00595546 | −7.3915716 | |
| LXS-196 | 7.64902 | 0.29120262 | −1.7799048 | | 0.15361 | 0.00997621 | −6.6472931 | |
| Mecillinam | 0.26076 | 0.00992728 | −6.6543852 | −3.4703236 | 15.96583 | 1.03690124 | 0.0522785 | −0.0660834 |
| (Mecillanim) | | | | | | | | |
| Mecillinam | 2.40334 | 0.09149654 | −3.4501389 | | 13.04952 | 0.84750142 | −0.2387123 | |
| Mecillinam | 21.24031 | 0.80863089 | −0.3064468 | | 15.27204 | 0.99184303 | −0.0118163 | |
| MK-0752 | 18.59155 | 0.70779107 | −0.4986045 | −0.4386106 | 12 | 0.77934031 | −0.3596747 | −0.3529502 |
| MK-0752 | 20.36855 | 0.77544249 | −0.3669083 | | 13.82066 | 0.89758312 | −0.1558825 | |
| MK-0752 | 19.22432 | 0.73188099 | −0.450319 | | 10.5659 | 0.68620265 | −0.5432934 | |
| MK-8245 | 25.73702 | 0.97982325 | −0.0294066 | 0.7763643 | 14.60771 | 0.94869811 | −0.075979 | −0.0573895 |
| MK-8245 | 25.12766 | 0.95662456 | −0.0639753 | | 12.93949 | 0.84035551 | −0.2509283 | |
| MK-8245 | 5.57692 | 0.21231657 | −2.2357111 | | 17.14096 | 1.11322009 | 0.15473885 | |
| Osanetant | 13.74536 | 0.52329381 | −0.9343069 | −0.838839 | 0.96283 | 0.06253102 | −3.9992842 | −3.9870052 |
| Osanetant | 14.36928 | 0.54704681 | −0.8702638 | | 1.30706 | 0.08488705 | −3.5583118 | |
| Osanetant | 16.0359 | 0.61049599 | −0.7119463 | | 0.7276 | 0.047254 | −4.4034197 | |
| Oxytetracycline | 22.00358 | 0.83768902 | −0.2555133 | −0.4729188 | 14.2002 | 0.92223236 | −0.1167978 | −0.1641768 |
| Oxytetracycline | 18.35809 | 0.69890311 | −0.5168356 | | 15.82871 | 1.02799598 | 0.03983463 | |
| Oxytetracycline | 16.78118 | 0.63886923 | −0.6464074 | | 11.54399 | 0.74972473 | −0.4155671 | |
| PCl 27483 | 15.22722 | 0.57970908 | −0.786599 | −0.6788627 | 16.06025 | 1.04303335 | 0.06078529 | 0.00618688 |
| (pci-27483) | | | | | | | | |
| PCl 27483 | 15.54068 | 0.59164268 | −0.757202 | | 16.23992 | 1.05470203 | 0.07683547 | |
| PCl 27483 | 18.66667 | 0.71065093 | −0.492787 | | 14.17795 | 0.92078733 | −0.1190601 | |
| Perindopril | 13.09469 | 0.49852243 | −1.0042697 | −0.8775044 | 13.79491 | 0.89591079 | −0.158573 | −0.1845786 |
| Perindopril | 13.6417 | 0.51934741 | −0.9452282 | | 12.14907 | 0.78902167 | −0.3418632 | |
| Perindopril | 16.36072 | 0.62286208 | −0.6830154 | | 14.83916 | 0.96372963 | −0.0532996 | |
| PIK-294 | 18.59181 | 0.70780097 | −0.4985844 | −0.485034 | 15.69413 | 1.01925568 | 0.027516 | −0.3548778 |
| PIK-294 | 21.04198 | 0.80108036 | −0.3199811 | | 11.57631 | 0.75182375 | −0.4115336 | |
| PIK-294 | 16.89639 | 0.64325535 | −0.6365366 | | 9.60657 | 0.62389894 | −0.6806157 | |
| Prostagladin | 15.47454 | 0.58912469 | −0.7633551 | −0.6806431 | 12.79249 | 0.8308086 | −0.267412 | −0.2281525 |
| F2a | | | | | | | | |
| (Prostaglandin | | | | | | | | |
| F2a) | | | | | | | | |
| Prostagladin | 17.47555 | 0.6653043 | −0.5879137 | | 12.64904 | 0.82149223 | −0.2836812 | |
| F2a | | | | | | | | |
| Prostagladin | 16.27425 | 0.61957012 | −0.6906605 | | 14.03807 | 0.91170282 | −0.1333645 | |
| F2a | | | | | | | | |
| PSI-7977 | 21.24746 | 0.8089031 | −0.3059612 | −0.2700306 | 10.86076 | 0.70535234 | −0.503584 | −0.4904569 |
| PSI-7977 | 23.14588 | 0.88117705 | −0.1824962 | | 11.30565 | 0.73424573 | −0.4456651 | |
| PSI-7977 | 21.01788 | 0.80016286 | −0.3216344 | | 10.7221 | 0.69634706 | −0.5221216 | |
| Rupatadine | 13.51835 | 0.51465141 | −0.9583325 | −0.7915615 | 13.56021 | 0.88066819 | −0.1833295 | −0.0960829 |
| Rupatadine | 16.83549 | 0.64093685 | −0.6417459 | | 16.38594 | 1.0641853 | 0.08974938 | |
| Rupatadine | 15.35433 | 0.58454823 | −0.774606 | | 13.45405 | 0.87377363 | −0.1946685 | |
| SCIO 469 | 12.59092 | 0.47934361 | −1.0608679 | −1.0388983 | 8.85049 | 0.5747953 | −0.7988798 | −0.8454228 |
| SCIO 469 | 12.94917 | 0.4929824 | −1.020392 | | 8.0449 | 0.52247624 | −0.9365627 | |
| SCIO 469 | 12.81485 | 0.48786876 | −1.035435 | | 8.83856 | 0.57402051 | −0.8008258 | |
| Tiaprofenic | 18.70804 | 0.71222591 | −0.4895932 | −0.4461538 | 13.32574 | 0.86544053 | −0.2084934 | −0.064607 |
| Acid | | | | | | | | |
| Tiaprofenic | 17.56458 | 0.66869373 | −0.5805825 | | 12.36133 | 0.8028069 | −0.3168751 | |
| Acid | | | | | | | | |
| Tiaprofenic | 21.80964 | 0.83030562 | −0.2682856 | | 19.37581 | 1.25836248 | 0.33154756 | |
| Acid | | | | | | | | |
| TMC207 | 19.10534 | 0.72735135 | −0.4592757 | −0.3555701 | 10.14096 | 0.65860491 | −0.6025148 | −0.558967 |
| (Bedaquiline) | | | | | | | | |
| TMC207 | 19.90305 | 0.75772063 | −0.4002621 | | 10.29152 | 0.66838303 | −0.581253 | |
| TMC207 | 22.75335 | 0.8662332 | −0.2071726 | | 10.93972 | 0.7104804 | −0.4931332 | |
| Xaliproden | 15.40541 | 0.58649287 | −0.7698145 | −0.6769005 | 15.9584 | 1.0364187 | 0.05160695 | −0.1429644 |
| Xaliproden | 14.11299 | 0.5372897 | −0.8962279 | | 13.77026 | 0.89430989 | −0.1611533 | |
| Xaliproden | 20.40033 | 0.77665237 | −0.3646591 | | 12.34017 | 0.80143266 | −0.3193468 | |

TABLE 9B
Selected Compound Testing Results

| | | Normalized | Average | | Normalized | Average |
|---|---|---|---|---|---|---|
| | Cell | Cell | Cell | Cell | Cell | Cell |
| | Number | Number | Number | Number | Number | Number |
| Treatment | (1 uM) | (1 uM) | (1 uM) | (5 uM) | (5 uM) | (5 uM) |
| ADX-47273 | 6477 | 1.1052901 | 1.04749716 | 5827 | 0.98326077 | 0.99484774 |
| ADX-47273 | 6480 | 1.10580205 | | 6142 | 1.03641457 | |
| ADX-47273 | 5458 | 0.93139932 | | 5718 | 0.96486787 | |
| Ampicillin | 3959 | 0.67559727 | 0.69397042 | 5481 | 0.92487597 | 0.96576783 |
| Ampicillin | 4296 | 0.7331058 | | 5912 | 0.99760386 | |
| Ampicillin | 3945 | 0.67320819 | | 5777 | 0.97482366 | |
| Bimiralisib | 3839 | 0.65511945 | 0.692719 | 2795 | 0.47163444 | 0.46370355 |
| Bimiralisib | 4405 | 0.75170648 | | 2791 | 0.47095947 | |
| Bimiralisib | 3934 | 0.67133106 | | 2658 | 0.44851676 | |
| CC-401 | 4691 | 0.80051195 | 0.84852105 | 4993 | 0.84252978 | 0.89354617 |
| CC-401 | 5161 | 0.88071672 | | 5513 | 0.93027572 | |
| CC-401 | 5065 | 0.86433447 | | 5380 | 0.90783301 | |
| Cefonicid | 5339 | 0.91109215 | 0.91222981 | 5781 | 0.97549863 | 0.98759182 |
| Cefonicid | 5337 | 0.91075085 | | 5781 | 0.97549863 | |
| Cefonicid | 5361 | 0.91484642 | | 5996 | 1.01177821 | |
| CH5132799 | 4526 | 0.77235495 | 0.65972696 | 3141 | 0.53001924 | 0.52388827 |
| CH5132799 | 3593 | 0.61313993 | | 3289 | 0.55499308 | |
| CH5132799 | 3479 | 0.59368601 | | 2884 | 0.48665249 | |
| Cintirorgon | 4247 | 0.72474403 | 0.74761092 | 5894 | 0.9945665 | 0.98742308 |
| Cintirorgon | 4539 | 0.77457338 | | 6058 | 1.02224022 | |
| Cintirorgon | 4357 | 0.74351536 | | 5603 | 0.94546252 | |
| Clidinium | 3871 | 0.6605802 | 0.68412969 | 5211 | 0.87931558 | 0.99850382 |
| Clidinium | 4256 | 0.72627986 | | 6271 | 1.05818231 | |
| Clidinium | 3900 | 0.66552901 | | 6270 | 1.05801357 | |
| DADLE | 4511 | 0.76979522 | 0.75341297 | 5991 | 1.01093449 | 0.98691686 |
| DADLE | 4466 | 0.76211604 | | 5664 | 0.9557558 | |
| DADLE | 4268 | 0.72832765 | | 5891 | 0.99406027 | |
| Daprodustat | 4292 | 0.73242321 | 0.74311718 | 5964 | 1.00637845 | 0.96948016 |
| Daprodustat | 4412 | 0.75290102 | | 5671 | 0.95693699 | |
| Daprodustat | 4360 | 0.7440273 | | 5601 | 0.94512504 | |
| Desogestrel | 5131 | 0.87559727 | 0.87684869 | 5708 | 0.96318045 | 0.9254947 |
| Desogestrel | 5212 | 0.8894198 | | 5824 | 0.98275455 | |
| Desogestrel | 5072 | 0.86552901 | | 4922 | 0.83054909 | |
| Etonogestrel | 4255 | 0.72610922 | 0.73401593 | 4904 | 0.82751173 | 0.58289179 |
| Etonogestrel | 4398 | 0.75051195 | | 5232 | 0.88285917 | |
| Etonogestrel | 4251 | 0.72542662 | | 227 | 0.03830448 | |
| GDC-0941 | 4447 | 0.75887372 | 0.78987486 | 5415 | 0.91373899 | 0.88257793 |
| GDC-0941 | 4698 | 0.80170648 | | 4385 | 0.73993453 | |
| GDC-0941 | 4741 | 0.80904437 | | 5891 | 0.99406027 | |
| GSK2269557 | 4831 | 0.82440273 | 0.89192264 | 4568 | 0.77081435 | 0.7914009 |
| GSK2269557 | 5665 | 0.96672355 | | 3831 | 0.64645135 | |
| GSK2269557 | 5184 | 0.88464164 | | 5671 | 0.95693699 | |
| Halofantrine lot −4 | 4727 | 0.80665529 | 0.80346985 | 119 | 0.02008032 | 0.66523798 |
| Halofantrine lot −4 | 4541 | 0.77491468 | | 5797 | 0.97819851 | |
| Halofantrine lot −4 | 4857 | 0.82883959 | | 5911 | 0.99743512 | |
| Halofantrine lot −5 | 3755 | 0.64078498 | 0.65819113 | 5286 | 0.89197125 | 0.86530998 |
| Halofantrine lot −5 | 3889 | 0.66365188 | | 5054 | 0.85282306 | |
| Halofantrine lot −5 | 3927 | 0.67013652 | | 5044 | 0.85113563 | |
| Ivacaftor | 3730 | 0.63651877 | 0.635438 | 4520 | 0.76271472 | 0.73498476 |
| Ivacaftor | 3662 | 0.62491468 | | 4563 | 0.76997064 | |
| Ivacaftor | 3779 | 0.64488055 | | 3984 | 0.67226891 | |
| Licofelone | 3716 | 0.63412969 | 0.62536974 | 4830 | 0.81502481 | 0.80613772 |
| Licofelone | 3591 | 0.61279863 | | 4798 | 0.80962505 | |
| Licofelone | 3687 | 0.62918089 | | 4704 | 0.79376329 | |
| Lonafarnib | 3801 | 0.64863481 | 0.65614334 | 3696 | 0.62367116 | 0.64155783 |
| Lonafarnib | 4072 | 0.69488055 | | 3879 | 0.65455098 | |
| Lonafarnib | 3662 | 0.62491468 | | 3831 | 0.64645135 | |
| Loratadine | 3981 | 0.67935154 | 0.67144482 | 4775 | 0.80574398 | 0.88128424 |
| Loratadine | 3790 | 0.64675768 | | 5571 | 0.94006277 | |
| Loratadine | 4033 | 0.68822526 | | 5322 | 0.89804597 | |
| LXS-196 | 5737 | 0.97901024 | 0.91251422 | 4550 | 0.76777699 | 0.53789387 |
| LXS-196 | 5507 | 0.93976109 | | 4362 | 0.73605346 | |
| LXS-196 | 4798 | 0.81877133 | | 651 | 0.10985117 | |
| Mecillinam | 6136 | 1.04709898 | 1.09698521 | 6088 | 1.02730249 | 0.99282283 |
| Mecillinam | 6699 | 1.14317406 | | 5755 | 0.97111134 | |
| Mecillinam | 6450 | 1.10068259 | | 5808 | 0.98005467 | |
| MK-0752 | 3905 | 0.66638225 | 0.69124005 | 5100 | 0.8605852 | 0.8745908 |
| MK-0752 | 4070 | 0.69453925 | | 5130 | 0.86564746 | |
| MK-0752 | 4177 | 0.71279863 | | 5319 | 0.89753974 | |
| MK-8245 | 5529 | 0.94351536 | 0.72627986 | 5812 | 0.98072964 | 0.94006277 |
| MK-8245 | 5679 | 0.96911263 | | 4892 | 0.82548682 | |
| MK-8245 | 1560 | 0.2662116 | | 6009 | 1.01397185 | |
| Osanetant | 4045 | 0.69027304 | 0.68822526 | 4466 | 0.75360265 | 0.77829525 |
| Osanetant | 3932 | 0.67098976 | | 4973 | 0.83915494 | |
| Osanetant | 4122 | 0.70341297 | | 4398 | 0.74212818 | |
| Oxytetracycline | 5031 | 0.85853242 | 0.83515358 | 6014 | 1.01481556 | 0.97600486 |
| Oxytetracycline | 4848 | 0.82730375 | | 5768 | 0.97330498 | |
| Oxytetracycline | 4803 | 0.81962457 | | 5570 | 0.93989403 | |
| PCl 27483 | 4643 | 0.79232082 | 0.77497156 | 5909 | 0.99709763 | 0.9438876 |
| PCl 27483 | 4781 | 0.81587031 | | 5702 | 0.962168 | |
| PCl 27483 | 4200 | 0.71672355 | | 5170 | 0.87239715 | |
| Perindopril | 4330 | 0.73890785 | 0.73646189 | 5734 | 0.96756775 | 0.99034795 |
| Perindopril | 4259 | 0.72679181 | | 6091 | 1.02780871 | |
| Perindopril | 4358 | 0.74368601 | | 5782 | 0.97566738 | |
| PIK-294 | 4346 | 0.74163823 | 0.71149033 | 5244 | 0.88488407 | 0.90383945 |
| PIK-294 | 3954 | 0.67474403 | | 5589 | 0.94310013 | |
| PIK-294 | 4208 | 0.71808874 | | 5236 | 0.88353414 | |
| Prostagladin F2a | 5047 | 0.8612628 | 0.89732651 | 5855 | 0.98798556 | 0.98579191 |
| Prostagladin F2a | 5419 | 0.92474403 | | 5787 | 0.97651109 | |
| Prostagladin F2a | 5309 | 0.9059727 | | 5884 | 0.99287908 | |
| PSI-7977 | 5916 | 1.00955631 | 1.03868032 | 5681 | 0.95862441 | 0.99102292 |
| PSI-7977 | 6135 | 1.04692833 | | 5997 | 1.01194695 | |
| PSI-7977 | 6209 | 1.05955631 | | 5941 | 1.00249738 | |
| Rupatadine | 4468 | 0.76245734 | 0.79499431 | 5730 | 0.96689278 | 0.91840752 |
| Rupatadine | 4936 | 0.84232082 | | 5462 | 0.92166987 | |
| Rupatadine | 4572 | 0.78020478 | | 5136 | 0.86665992 | |
| SCIO 469 | 3987 | 0.68037543 | 0.74385666 | 5672 | 0.95710573 | 0.94996231 |
| SCIO 469 | 4564 | 0.77883959 | | 5345 | 0.90192704 | |
| SCIO 469 | 4526 | 0.77235495 | | 5872 | 0.99085417 | |
| Tiaprofenic Acid | 5511 | 0.94044369 | 0.86382253 | 6146 | 1.03708953 | 0.96790523 |
| Tiaprofenic Acid | 5420 | 0.92491468 | | 5679 | 0.95828693 | |
| Tiaprofenic Acid | 4255 | 0.72610922 | | 5383 | 0.90833924 | |
| TMC207 | 3465 | 0.59129693 | 0.60483504 | 5108 | 0.86193514 | 0.81913087 |
| TMC207 | 3507 | 0.59846416 | | 4528 | 0.76406466 | |
| TMC207 | 3661 | 0.62474403 | | 4927 | 0.8313928 | |
| Xaliproden | 4440 | 0.75767918 | 0.71877133 | 5577 | 0.94107523 | 0.913514 |
| Xaliproden | 4549 | 0.77627986 | | 5737 | 0.96807398 | |
| Xaliproden | 3647 | 0.62235495 | | 4927 | 0.8313928 | |

TABLE 9C
Selected Compound Testing Results

| | HIT | | HIT | | |
|---|---|---|---|---|---|
| | (2 Std, | Toxic | (2 Stdv, | Toxic | |
| Treatment | 1 uM) | (1 uM) | 5 uM) | (5 uM) | HIT |
| ADX-47273 | HIT | 0 | 0 | 0 | Yes |
| ADX-47273 | | | | | |
| ADX-47273 | | | | | |
| Ampicillin | HIT | 0 | 0 | 0 | Yes |
| Ampicillin | | | | | |
| Ampicillin | | | | | |
| Bimiralisib | HIT | 0 | HIT | TOXIC | Yes |
| Bimiralisib | | | | | |
| Bimiralisib | | | | | |
| CC-401 | 0 | 0 | HIT | 0 | Yes |
| CC-401 | | | | | |
| CC-401 | | | | | |
| Cefonicid | HIT | 0 | 0 | 0 | Yes |
| Cefonicid | | | | | |
| Cefonicid | | | | | |
| CH5132799 | HIT | 0 | HIT | TOXIC | Yes |
| CH5132799 | | | | | |
| CH5132799 | | | | | |
| Cintirorgon | HIT | 0 | 0 | 0 | Yes |
| Cintirorgon | | | | | |
| Cintirorgon | | | | | |
| Clidinium | HIT | 0 | 0 | 0 | Yes |
| Clidinium | | | | | |
| Clidinium | | | | | |
| DADLE | HIT | 0 | 0 | 0 | Yes |
| DADLE | | | | | |
| DADLE | | | | | |
| Daprodustat | HIT | 0 | 0 | 0 | Yes |
| Daprodustat | | | | | |
| Daprodustat | | | | | |
| Desogestrel | HIT | 0 | 0 | 0 | Yes |
| Desogestrel | | | | | |
| Desogestrel | | | | | |
| Etonogestrel | HIT | 0 | HIT | TOXIC | Yes |
| Etonogestrel | | | | | |
| Etonogestrel | | | | | |
| GDC-0941 | HIT | 0 | HIT | 0 | Yes |
| GDC-0941 | | | | | |
| GDC-0941 | | | | | |
| GSK2269557 | HIT | 0 | HIT | 0 | Yes |
| GSK2269557 | | | | | |
| GSK2269557 | | | | | |
| Halofantrine lot −4 | HIT | 0 | HIT | 0 | Yes |
| Halofantrine lot −4 | | | | | |
| Halofantrine lot −4 | | | | | |
| Halofantrine lot −5 | HIT | 0 | HIT | 0 | Yes |
| Halofantrine lot −5 | | | | | |
| Halofantrine lot −5 | | | | | |
| Ivacaftor | HIT | TOXIC | HIT | 0 | Yes |
| Ivacaftor | | | | | |
| Ivacaftor | | | | | |
| Licofelone | HIT | TOXIC | HIT | 0 | Yes |
| Licofelone | | | | | |
| Licofelone | | | | | |
| Lonafarnib | HIT | 0 | HIT | TOXIC | Yes |
| Lonafarnib | | | | | |
| Lonafarnib | | | | | |
| Loratadine | 0 | 0 | HIT | 0 | Yes |
| Loratadine | | | | | |
| Loratadine | | | | | |
| LXS-196 | HIT | 0 | HIT | TOXIC | Yes |
| LXS-196 | | | | | |
| LXS-196 | | | | | |
| Mecillinam | HIT | 0 | 0 | 0 | Yes |
| Mecillinam | | | | | |
| Mecillinam | | | | | |
| MK-0752 | HIT | 0 | 0 | 0 | Yes |
| MK-0752 | | | | | |
| MK-0752 | | | | | |
| MK-8245 | HIT | 0 | 0 | 0 | Yes |
| MK-8245 | | | | | |
| MK-8245 | | | | | |
| Osanetant | HIT | 0 | HIT | 0 | Yes |
| Osanetant | | | | | |
| Osanetant | | | | | |
| Oxytetracycline | HIT | 0 | 0 | 0 | Yes |
| Oxytetracycline | | | | | |
| Oxytetracycline | | | | | |
| PCI 27483 | HIT | 0 | 0 | 0 | Yes |
| PCI 27483 | | | | | |
| PCI 27483 | | | | | |
| Perindopril | HIT | 0 | 0 | 0 | Yes |
| Perindopril | | | | | |
| Perindopril | | | | | |
| PIK-294 | HIT | 0 | 0 | 0 | Yes |
| PIK-294 | | | | | |
| PIK-294 | | | | | |
| Prostagladin F2a | HIT | 0 | 0 | 0 | Yes |
| Prostagladin F2a | | | | | |
| Prostagladin F2a | | | | | |
| PSI-7977 | 0 | 0 | HIT | 0 | Yes |
| PSI-7977 | | | | | |
| PSI-7977 | | | | | |
| Rupatadine | HIT | 0 | 0 | 0 | Yes |
| Rupatadine | | | | | |
| Rupatadine | | | | | |
| SCIO 469 | HIT | 0 | HIT | 0 | Yes |
| SCIO 469 | | | | | |
| SCIO 469 | | | | | |
| Tiaprofenic Acid | HIT | 0 | 0 | 0 | Yes |
| Tiaprofenic Acid | | | | | |
| Tiaprofenic Acid | | | | | |
| TMC207 | HIT | TOXIC | HIT | 0 | Yes |
| TMC207 | | | | | |
| TMC207 | | | | | |
| Xaliproden | HIT | 0 | 0 | 0 | Yes |
| Xaliproden | | | | | |
| Xaliproden | | | | | |

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method for preventing, delaying the onset of, or treating an infection or a disease caused by a RNA virus, comprising administering to a subject in need thereof a composition comprising a compound or a pharmaceutically acceptable salt, ester, solvate, stereoisomer, tautomer, or prodrug thereof, thereby preventing, delaying the onset of, or treating the infection or the disease, wherein the compound is selected from the compounds listed in Tables 2-8.

2. A method for preventing, delaying the onset of, or treating an inflammatory effect of an infection or a disease caused by a RNA virus, comprising administering to a subject in need thereof a composition comprising a compound or a pharmaceutically acceptable salt, ester, solvate, stereoisomer, tautomer, or prodrug thereof, thereby preventing, delaying the onset of, or treating the inflammatory effect, wherein the compound is selected from the compounds listed in Tables 2-8.

3. A method for imparting resistance to an RNA virus to a cell, comprising contacting the cell with a composition comprising a compound or a pharmaceutically acceptable salt, ester, solvate, stereoisomer, tautomer, or prodrug thereof, thereby imparting resistance to the RNA virus to the cell, wherein the compound is selected from the compounds listed in Tables 2-8.

4. The method of claim 3, the method comprising contacting a plurality of cells with the composition, optionally the method comprises determining the infection rate of the plurality of cells after being contacted with the composition.

5. The method of any one of claims 3-4, wherein the cell expresses angiotensin-converting enzyme 2 (ACE2).

6. The method of any one of claims 3-5, wherein the cell is a lung cell, an enterocyte, an endothelial cell, an epithelial cell, a kidney cell, an arterial smooth muscle cell, a cell of the respiratory tract, or any combination thereof.

7. The method of any one of claims 3-6, wherein the cell is the cell of a subject, optionally the cell is in a subject.

8. The method of any one of claims 3-7, wherein contacting the cell with the composition is in a subject.

9. The method of any one of claims 3-8, wherein contacting the cell with the composition occurs in vitro, ex vivo, and/or in vivo.

10. The method of any one of claims 1-9, wherein the inflammatory effect comprises respiratory failure, a sequela of respiratory failure, acute lung injury, or acute respiratory distress syndrome.

11. The method of claim 10, wherein the sequela of respiratory failure comprises multi-organ failure.

12. The method of any one of claims 1-11, wherein the composition comprises a therapeutically or prophylactically effective amount of the compound.

13. The method of any one of claims 1-12, wherein the subject in need thereof is a subject that is suffering from the infection or the disease, or a subject that is at a risk for the infection or the disease.

14. The method of claim 13, wherein the infection or the disease is in the respiratory tract of the subject.

15. The method of any one of claims 1-14, wherein the subject has been exposed to the RNA virus, is suspected to have been exposed to the RNA virus, or is at a risk of being exposed to the RNA virus.

16. The method of any one of claims 1-15, wherein the subject is a mammal.

17. The method of any one of claims 1-15, wherein the subject is a human.

18. The method of any one of claims 1-17, wherein the RNA virus is a double-stranded RNA virus.

19. The method of any one of claims 1-17, wherein the RNA virus is a positive-sense single-stranded ssRNA virus.

20. The method of claim 19, wherein the positive-sense single-stranded ssRNA virus is a coronavirus.

21. The method of claim 20, wherein the coronavirus is an alpha coronavirus, a beta coronavirus, a gamma coronavirus, or a delta coronavirus.

22. The method of claim 20, wherein the coronavirus is Middle East respiratory coronavirus (MERS-CoV), severe acute respiratory syndrome coronavirus (SARS-CoV), or SARS-CoV-2.

23. The method of any one of claims 1-22, wherein the disease is common cold, influenza, SARS, coronaviruses, COVID-19, hepatitis C, hepatitis E, West Nile fever, Ebola virus disease, rabies, polio, or measles.

24. The method of any one of claims 1-23, wherein the composition is a pharmaceutical composition comprising the compound and one or more pharmaceutically acceptable excipients.

25. The method of any one of claims 1-24, comprising administering to the subject one or more additional antiviral agents.

26. The method of claim 25, wherein at least one of the one or more additional antiviral agents is co-administered to the subject with the composition.

27. The method of claim 25, wherein at least one of the one or more additional antiviral agents is administered to the subject before the administration of the composition, after the administration of the composition, or both.

28. The method of any one of claims 1-27, wherein the composition comprises one or more additional therapeutic agents.

29. The method of claim 28, wherein the one or more additional therapeutic agents comprise one or more antiviral agents.

30. The method of any one of claims 25-29, wherein the antiviral agent is selected from the group consisting of a nucleoside or a non-nucleoside analogue reverse-transcriptase inhibitor, a nucleotide analogue reverse-transcriptase inhibitor, a NS3/4A serine protease inhibitor, a NS5B polymerase inhibitor, and interferon alpha.

31. The method of any one of claims 1-30, wherein the composition is administered to the subject by intravenous administration, nasal administration, pulmonary administration, oral administration, parenteral administration, or nebulization.

32. The method of any one of claims 1-31, wherein the composition is aspirated into at least one lung of the subject.

33. The method of any one of claims 1-32, wherein the composition is in the form of powder, pill, tablet, microtablet, pellet, micropellet, capsule, capsule containing microtablets, liquid, aerosols, or nanoparticles.

34. The method of any one of claims 1-33, wherein the composition is in a formulation for administration to the lungs.

35. The method of any one of claims 1-34, wherein the composition is administered to the subject once, twice, or three times a day.

36. The method of any one of claims 1-35, wherein the composition is administered to the subject once every day, every two days, or every three days.

37. The method of any one of claims 1-36, wherein the composition is administered to the subject over the course of at least two weeks, at least three weeks, at least four weeks, or at least five weeks.

38. The method of any one of claims 1-37, further comprising measuring the viral titer of the RNA virus in the subject before administering the composition to the subject, after administering the composition to the subject, or both.

39. The method of claim 38, wherein the viral titer is lung bulk virus titer.

40. The method of any one of claims 1-39, wherein administrating the composition results in reduction of the viral titer of the RNA virus in the subject as compared to that in the subject before administration of the composition.

41. The method of any one of claims 1-40, further comprising determining global virus distribution in the lungs of the subject.

42. The method of any one of claims 1-41, further comprising measuring a neutrophil density within the lungs of the subject.

43. The method of claim 42, wherein administering the composition results in reduction of the neutrophil density within the lungs of the subject as compared to that in the subject before administration of the composition.

44. The method of any one of claims 1-43, further comprising measuring a total necrotized cell count within the lungs of the subject.

45. The method of claim 44, wherein administering the composition results in reduction of the total necrotized cell count in the subject as compared to that in the subject before administration of the composition.

46. The method of any one of claims 1-45, further comprising measuring a total protein level within the lungs of the subject.

47. The method of claim 46, wherein administering the composition results in reduction of the total protein level within the lungs of the subject as compared to that in the subject before administration of the composition.

48. A method for preventing, delaying the onset of, or treating an infection or a disease caused by a RNA virus, comprising administering to a subject in need thereof (1) a first compound selected from the compounds listed in Tables 2-8, or a pharmaceutically acceptable salt, ester, solvate, stereoisomer, tautomer, or prodrug thereof, and (2) a second compound selected from the compounds listed in Tables 2-8, or a pharmaceutically acceptable salt, ester, solvate, stereoisomer, tautomer, or prodrug thereof, thereby preventing, delaying the onset of, or treating the inflammatory effect, wherein the first compound and the second compound are different.

49. A method for preventing, delaying the onset of, or treating an inflammatory effect of an infection or a disease caused by a RNA virus, comprising administering to a subject in need thereof (1) a first compound selected from the compounds listed in Tables 2-8, or a pharmaceutically acceptable salt, ester, solvate, stereoisomer, tautomer, or prodrug thereof, and (2) a second compound selected from the compounds listed in Tables 2-8, or a pharmaceutically acceptable salt, ester, solvate, stereoisomer, tautomer, or prodrug thereof, thereby preventing, delaying the onset of, or treating the inflammatory effect, wherein the first compound and the second compound are different.

50. A method for imparting resistance to an RNA virus to a cell, comprising contacting the cell with (1) a first compound selected from the compounds listed in Tables 2-8, or a pharmaceutically acceptable salt, ester, solvate, stereoisomer, tautomer, or prodrug thereof, and (2) a second compound selected from the compounds listed in Tables 2-8, or a pharmaceutically acceptable salt, ester, solvate, stereoisomer, tautomer, or prodrug thereof, thereby imparting resistance to the RNA virus to the cell, wherein the first compound and the second compound are different.

51. The method of claim 50, the method comprising contacting a plurality of cells with the first compound and the second compound, optionally the method comprises determining the infection rate of the plurality of cells after being contacted with the first compound and the second compound.

52. The method of any one of claims 50-51, wherein the cell expresses angiotensin-converting enzyme 2 (ACE2).

53. The method of any one of claims 50-52, wherein the cell is a lung cell, an enterocyte, an endothelial cell, an epithelial cell, a kidney cell, an arterial smooth muscle cell, a cell of the respiratory tract, or any combination thereof.

54. The method of any one of claims 50-53, wherein the cell is the cell of a subject, optionally the cell is in a subject.

55. The method of any one of claims 50-54, wherein contacting the cell with the first compound and the second compound is in a subject.

56. The method of any one of claims 50-55, wherein contacting the cell with the first compound and the second compound occurs in vitro, ex vivo, and/or in vivo.

57. The method of claim 49, wherein the inflammatory effect comprises respiratory failure, a sequela of respiratory failure, acute lung injury, or acute respiratory distress syndrome, optionally the sequela of respiratory failure comprises multi-organ failure.

58. The method of any one of claims 48-57, further comprising administering to the subject (3) a third compound selected from the compounds listed in Tables 2-8, or a pharmaceutically acceptable salt, ester, solvate, stereoisomer, tautomer, or prodrug thereof, wherein the first, second and third compounds are different.

59. The method of any one of claims 48-58, wherein the first compound, the second compound, and/or the third compound is administered in a therapeutically or prophylactically effective amount.

60. The method of any one of claims 48-59, wherein the subject in need thereof is a subject that is suffering from the infection or the disease, or a subject that is at a risk for the infection or the disease, optionally the infection or the disease is in the respiratory tract of the subject.

61. The method of any one of claims 48-60, wherein the subject has been exposed to the RNA virus, is suspected to have been exposed to the RNA virus, or is at a risk of being exposed to the RNA virus.

62. The method of any one of claims 48-61, wherein the subject is a mammal, optionally the subject is a human.

63. The method of any one of claims 48-62, wherein the RNA virus is a double-stranded RNA virus.

64. The method of any one of claims 48-62, wherein the RNA virus is a positive-sense single-stranded ssRNA virus, optionally the positive-sense single-stranded ssRNA virus is a coronavirus, and further optionally the coronavirus is an alpha coronavirus, a beta coronavirus, a gamma coronavirus, or a delta coronavirus.

65. The method of claim 64, wherein the coronavirus is Middle East respiratory coronavirus (MERS-CoV), severe acute respiratory syndrome coronavirus (SARS-CoV), or SARS-CoV-2.

66. The method of any one of claims 48-65, wherein the disease is common cold, influenza, SARS, coronaviruses, COVID-19, hepatitis C, hepatitis E, West Nile fever, Ebola virus disease, rabies, polio, or measles.

67. The method of any one of claims 48-66, wherein the first, second and/or third compound is in a pharmaceutical composition comprising one or more pharmaceutically acceptable excipients.

68. The method of any one of claims 48-67, comprising administering to the subject one or more additional therapeutic agents.

69. The method of claim 68, wherein the therapeutic agent is selected from the group consisting of a nucleoside or a non-nucleoside analogue reverse-transcriptase inhibitor, a nucleotide analogue reverse-transcriptase inhibitor, a NS3/4A serine protease inhibitor, a NS5B polymerase inhibitor, and interferon alpha.

70. The method of claim 68 or 69, wherein at least one of the one or more additional therapeutic agents is administered to the subject before the administration of the first, second or third compound; after the administration of the first, second or third compound; or both.

71. The method of any one of claims 48-70, wherein at least two of the first, second and third compounds are co-administered in a single composition or in separate compositions to the subject.

72. The method of any one of claims 49-70, wherein the first, second and third compounds are co-administered in a single composition or in separate compositions to the subject.

73. The method of any one of claims 48-72, wherein the first, second and/or third compound is administered to the subject by intravenous administration, nasal administration, pulmonary administration, oral administration, parenteral administration, nebulization, or a combination thereof.

74. The method of any one of claims 48-73, wherein the first, second and/or third compound is aspirated into at least one lung of the subject.

75. The method of any one of claims 48-74, wherein at least one of the first, second and third compounds is in a composition in the form of powder, pill, tablet, microtablet, pellet, micropellet, capsule, capsule containing microtablets, liquid, aerosols, or nanoparticles.

76. The method of any one of claims 48-75, wherein at least one of the first, second and third compounds is in a composition in a formulation for administration to the lungs.

77. The method of any one of claims 48-76, wherein at least one of the first, second and third compounds is administered to the subject once, twice, or three times a day.

78. The method of any one of claims 48-77, wherein at least one of the first, second and third compounds is administered to the subject once every day, every two days, or every three days.

79. The method of any one of claims 48-78, wherein at least one of the first, second and third compounds is administered to the subject over the course of at least two weeks, at least three weeks, at least four weeks, or at least five weeks.

80. The method of any one of claims 48-79, further comprising measuring the viral titer of the RNA virus in the subject before administering the first, second and/or the third compound to the subject, after administering the first, second and/or the third compound to the subject, or both, optionally the viral titer is lung bulk virus titer.

81. The method of any one of claims 48-80, wherein administrating the first, second and/or the third compound results in reduction of the viral titer of the RNA virus in the subject as compared to that in the subject before administration of the first, second and/or the third compound.

82. The method of any one of claims 48-81, further comprising determining global virus distribution in the lungs of the subject.

83. The method of any one of claims 48-82, further comprising measuring a neutrophil density within the lungs of the subject.

84. The method of claim 83, wherein administering the first, second and/or the third compound results in reduction of the neutrophil density within the lungs of the subject as compared to that in the subject before administration of the first, second and/or the third compound.

85. The method of any one of claims 48-84, further comprising measuring a total necrotized cell count within the lungs of the subject, optionally administering the first, second and/or the third compound results in reduction of the total necrotized cell count in the subject as compared to that in the subject before administration of the first, second and/or the third compound.

86. The method of any one of claims 48-85, further comprising measuring a total protein level within the lungs of the subject.

87. The method of claim 86, wherein administering the first, second and/or the third compound results in reduction of the total protein level within the lungs of the subject as compared to that in the subject before administration of the first, second and/or the third compound.

88. A kit, comprising
a first compound selected from the compounds listed in Tables 2-8, or a pharmaceutically acceptable salt, ester, solvate, stereoisomer, tautomer, or prodrug thereof, and
a label indicating that the kit is for preventing, delaying the onset of, or treating an infection or a disease caused by a RNA virus.

89. A kit, comprising
a first compound selected from the compounds listed in Tables 2-8, or a pharmaceutically acceptable salt, ester, solvate, stereoisomer, tautomer, or prodrug thereof, and
a label indicating that the kit is for preventing, delaying the onset of, or treating an inflammatory effect of an infection or a disease caused by a RNA virus.

90. A kit, comprising
a first compound selected from the compounds listed in Tables 2-8, or a pharmaceutically acceptable salt, ester, solvate, stereoisomer, tautomer, or prodrug thereof; and
a label indicating that the kit is for imparting resistance to an RNA virus to a cell.

91. The kit of any one of claims 88-90, further comprising a second compound selected from the compounds listed in Tables 2-8, or a pharmaceutically acceptable salt, ester, solvate, stereoisomer, tautomer, or prodrug thereof, wherein the first compound and the second compound is different.

92. The kit of any one of claims 88-91, further comprising a third compound selected from the compounds listed in Tables 2-8, or a pharmaceutically acceptable salt, ester, solvate, stereoisomer, tautomer, or prodrug thereof, wherein the first, second and third compound are different.

93. The kit of any one of claims 88-92, wherein the RNA virus is a coronavirus, optionally the coronavirus is Middle East respiratory coronavirus (MERS-CoV), severe acute respiratory syndrome coronavirus (SARS-CoV), or SARS-CoV-2.

94. A composition comprising a compound selected from the compounds listed in Tables 2-8, or a pharmaceutically acceptable salt, ester, solvate, stereoisomer, tautomer, or prodrug thereof, for use in preventing, delaying the onset of, or treating an infection or a disease caused by a RNA virus.

95. A composition comprising a compound selected from the compounds listed in Tables 2-8, or a pharmaceutically acceptable salt, ester, solvate, stereoisomer, tautomer, or prodrug thereof, for use in preventing, delaying the onset of, or treating an inflammatory effect of an infection or a disease caused by a RNA virus.

96. A composition comprising a compound selected from the compounds listed in Tables 2-8, or a pharmaceutically acceptable salt, ester, solvate, stereoisomer, tautomer, or prodrug thereof, for use in imparting resistance to an RNA virus to a cell.

97. The composition of any one of claims 94-96, wherein the RNA virus is a coronavirus, optionally the coronavirus is Middle East respiratory coronavirus (MERS-CoV), severe acute respiratory syndrome coronavirus (SARS-CoV), or SARS-CoV-2.
